SEQUENCE LISTING (39 sequences)

SEQ ID NO: 1 - protein, 177 aa, Homo sapiens
  source 1..177: note = Amino acid sequence of human IL-7 (Accession number: P13232)
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 154 aa, Rattus norvegicus
  source 1..154: note = amino acid sequence of rat IL-7 (Accession number: P56478)
MFHVSFRYIFGIPPLILVLLPVTSSDCHIKDKDGKAFGSVLMISINQLDKMTGTDSDCPNNEPNFFKKHLCDDTKEAAFLNRAARKLRQFLKMNISEEFNDHLLRVSDGTQTLVNCTSKEEKTIKEQKKNDPCFLKRLLREIKTCWNKILKGSI

SEQ ID NO: 3 - protein, 154 aa, Mus musculus
  source 1..154: note = amino acid sequence of mouse IL-7 (Accession number: P10168)
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 4 - protein, 177 aa, Chlorocebus sabaeus
  source 1..177: note = amino acid sequence of monkey IL-7 (Accession number : NP_001279008)
MFHVSFRYIFGLPPLILVLLPVASSDCDIEGKDGKQYESVLMVSIDQLLDSMKEIGSNCLNNEFNFFKRHLCDDNKEGMFLFRAARKLKQFLKMNSTGDFDLHLLKVSEGTTILLNCTGKVKGRKPAALGEPQPTKSLEENKSLKEQKKLNDSCFLKRLLQKIKTCWNKILMGTKEH

SEQ ID NO: 5 - protein, 176 aa, Bos taurus
  source 1..176: note = amino acid sequence of cow IL-7 (Accession number: P26895)
MFHVSFRYIFGIPPLILVLLPVASSDCDISGKDGGAYQNVLMVNIDDLDNMINFDSNCLNNEPNFFKKHSCDDNKEASFLNRASRKLRQFLKMNISDDFKLHLSTVSQGTLTLLNCTSKGKGRKPPSLSEAQPTKNLEENKSSKEQKKQNDLCFLKILLQKIKTCWNKILRGIKEH

SEQ ID NO: 6 - protein, 176 aa, Ovis aries
  source 1..176: note = amino acid sequence of sheep IL-7 (Accession number : Q28540)
MFHVSFRYIFGIPPLILVLLPVASSDCDFSGKDGGAYQNVLMVSIDDLDNMINFDSNCLNNEPNFFKKHSCDDNKEASFLNRAARKLKQFLKMNISDDFKLHLSTVSQGTLTLLNCTSKGKGRKPPSLGEAQPTKNLEENKSLKEQRKQNDLCFLKILLQKIKTCWNKILRGITEH

SEQ ID NO: 7 - protein, 384 aa, Homo sapiens
  source 1..384: note = amino acid sequence of human IgD constant region (Genbank accession No. P01880)
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 8 - protein, 327 aa, Homo sapiens
  source 1..327: note = amino acid sequence of Partial human IgG4 constant region (Genbank accession No. AAH25985)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 245 aa, synthetic construct
  source 1..245: note = amino acid sequence of hyFc
RNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 245 aa, synthetic construct
  source 1..245: note = amino acid sequence of hyFcM1
RNTGRGGEEKKGGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 245 aa, synthetic construct
  source 1..245: note = amino acid sequence of hyFcM2
RNTGRGGEEKKGSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 245 aa, synthetic construct
  source 1..245: note = amino acid sequence of hyFcM3
RNTGRGGEEKKSGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 245 aa, synthetic construct
  source 1..245: note = amino acid sequence of hyFcM4
RNTGRGGEEKKSSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 243 aa, synthetic construct
  source 1..243: note = amino acid sequence of mouse IgG Fc variant
ASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 15 - protein, 153 aa, synthetic construct
  source 1..153: note = amino acid sequence of modified IL-7(M)
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 16 - protein, 154 aa, synthetic construct
  source 1..154: note = amino acid sequence of modified IL-7(MM)
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 17 - protein, 155 aa, synthetic construct
  source 1..155: note = amino acid sequence of modified IL-7(MMM)
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 18 - protein, 155 aa, synthetic construct
  source 1..155: note = amino acid sequence of modified IL-7(MGM)
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 19 - protein, 155 aa, synthetic construct
  source 1..155: note = amino acid sequence of modified IL-7(DDD)
DDDDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 20 - protein, 156 aa, synthetic construct
  source 1..156: note = amino acid sequence of modified IL-7(MMMM)
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 21 - protein, 398 aa, synthetic construct
  source 1..398: note = amino acid sequence of modified IL-7(M) fused hyFc
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 399 aa, synthetic construct
  source 1..399: note = amino acid sequence of modified IL-7(MM) fused hyFc
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 400 aa, synthetic construct
  source 1..400: note = amino acid sequence of modified IL-7(MMM) fused hyFc
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 400 aa, synthetic construct
  source 1..400: note = amino acid sequence of modified IL-7(MGM) fused hyFc
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 401 aa, synthetic construct
  source 1..401: note = amino acid sequence of modified IL-7(MMMM) fused hyFc
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 397 aa, synthetic construct
  source 1..397: note = amino acid sequence of human IL-7 fused hyFc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 395 aa, synthetic construct
  source 1..395: note = amino acid sequence of human IL-7 fused nonlytic mouse Fc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 28 - DNA, 531 bp, synthetic construct
  source 1..531: mol_type = other DNA; note = nucleotide sequence of human IL-7
atgttccacgtgagcttcaggtacatcttcggcctgccacccctgatcctggtgctgctgcctgtggccagctccgactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacac

SEQ ID NO: 29 - DNA, 534 bp, synthetic construct
  source 1..534: mol_type = other DNA; note = nucleotide sequence of modified IL-7(M)
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 30 - DNA, 537 bp, synthetic construct
  source 1..537: mol_type = other DNA; note = nucleotide sequence of modified IL-7(MM)
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 31 - DNA, 540 bp, synthetic construct
  source 1..540: mol_type = other DNA; note = nucleotide sequence of modified IL-7(MMM)
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 32 - DNA, 540 bp, synthetic construct
  source 1..540: mol_type = other DNA; note = nucleotide sequence of modified IL-7(MGM)
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcaccaaggagcac

SEQ ID NO: 33 - DNA, 540 bp, synthetic construct
  source 1..540: mol_type = other DNA; note = nucleotide sequence of modified IL-7(DDD)
atgttccacgtgagcttcagatacatcttcggcctgccccccctgatcctggtgctgctgcccgtggccagcagcgacgatgacgactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 34 - DNA, 543 bp, synthetic construct
  source 1..543: mol_type = other DNA; note = nucleotide sequence of modified IL-7(MMMM)
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 35 - DNA, 1284 bp, synthetic construct
  source 1..1284: mol_type = other DNA; note = nucleotide sequence of modified IL-7(M) fused hyFc
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtgactcgagtctaga

SEQ ID NO: 36 - DNA, 1272 bp, synthetic construct
  source 1..1272: mol_type = other DNA; note = nucleotide sequence of modified IL-7(MM) fused hyFc
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 37 - DNA, 1275 bp, synthetic construct
  source 1..1275: mol_type = other DNA; note = nucleotide sequence of modified IL-7(MMM) fused hyFc
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 38 - DNA, 1275 bp, synthetic construct
  source 1..1275: mol_type = other DNA; note = nucleotide sequence of modified IL-7(MGM) fused hyFc
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacaccgcaatactgggcggggcggggaggaaaagaaaaaggagaaggaaaaggaggaacaggaggaaagagagactaagaccccagaatgtcccagccatactcagcccctgggggtgttcctgtttccccctaaacctaaggataccctgatgatcagcaggacacccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 39 - DNA, 1278 bp, synthetic construct
  source 1..1278: mol_type = other DNA; note = nucleotide sequence of modified IL-7(MMMM) fused hyFc
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

(19) United States
(12) Patent Application Publication
(10) Pub. No.: US 2026/0151458 A1
(43) Pub. Date: Jun. 4, 2026

(54) USE OF INTERLEUKIN-7 AND CHIMERIC ANTIGEN RECEPTOR (CAR)-BEARING IMMUNE EFFECTOR CELLS FOR TREATING TUMOR

(71) Applicants: WASHINGTON UNIVERSITY, St. Louis, MO (US); NEOIMMUNETECH, INC., Rockville, MD (US)

(72) Inventors: John F. DIPERSIO, St. Louis, MO (US); Matthew COOPER, St. Louis, MO (US); Karl STASER, St. Louis, MO (US); Jaehan PARK, Rockville, MD (US); Byung Ha LEE, Rockville, MD (US); Dong Hoon CHOI, Rockville, MD (US)

(73) Assignees: WASHINGTON UNIVERSITY, St. Louis, MO (US); NEOIMMUNETECH, INC., Rockville, MD (US)

(21) Appl. No.: 19/293,499

(22) Filed: Aug. 7, 2025

Related U.S. Application Data
(63) Continuation of application No. 17/264,760, now abandoned.
(60) Provisional application No. 62/712,803, filed on Jul. 31, 2018.
(60) Provisional application No. 62/804,604, filed on Feb. 12, 2019.

(51) Int. Cl. A61K 38/20; A61K 39/395; A61K 40/11; A61K 40/15; A61K 40/31; A61K 40/42; A61K 45/06; A61P 35/00; A61P 35/02; C07K 14/54; C07K 14/705; C07K 14/725; C07K 14/73

(52) U.S. Cl. CPC A61K 38/2046; A61K 39/3955; A61K 40/11; A61K 40/15; A61K 40/31; A61K 40/421; A61K 40/4211; A61K 40/4215; A61K 40/4255; A61K 45/06; A61P 35/00; A61P 35/02; C07K 14/5418; C07K 14/70503; C07K 14/70507; C07K 14/7051; C07K 14/70514; C07K 14/70578; C07K 14/70596; A61K 2239/31; A61K 2239/38; A61K 2239/48; A61K 2239/49; A61K 2239/54; C07K 2319/30; C07K 2319/31

(57) ABSTRACT

Disclosed herein are therapeutic uses for treating cancer in a subject in need thereof, comprising administering to the subject a population of chimeric antigen receptor (CAR)-bearing immune effector cells and an IL-7 protein (e.g., modified IL-7 protein).

### CROSS-REFERENCE TO RELATED APPLICATIONS

This PCT application claims the priority benefit of U.S. Provisional Application Nos. 62/712,803, filed Jul. 31, 2018; and 62/804,604, filed Feb. 12, 2019, each of which is herein incorporated by reference in its entirety.

### REFERENCE TO SEQUENCE LISTING SUBMITTED ELECTRONICALLY

The content of the electronically submitted sequence listing in ASCII text file (Name: 4241_003PC03_Seglisting_ST25.txt; Size: 73,691 bytes; and Date of Creation: Jul. 25, 2019) filed with the application is incorporated herein by reference in its entirety.

### BACKGROUND OF THE DISCLOSURE

Chimeric antigen receptor T cell (CAR-T) immunotherapy is increasingly well known. T cells are genetically modified to express chimeric antigen receptors (CARs), which are fusion proteins comprised of an antigen recognition moiety and T cell activation domains. The CARs are designed to recognize antigens that are overexpressed on cancer cells. CAR-Ts demonstrate exceptional clinical efficacy against B cell malignancies, and two therapies, KYMRIAH™ (tisagenlecleucel, Novartis) and YESCARTA™ (axicabtagene ciloleucel, Kite/Gilead), were recently approved by the FDA. Recent disclosures have also shown promise in expanding the react of CAR-T therapy to T-cell malignancies as well, and in enabling “off-the-shelf” use of pre-engineered cells from donors to treat malignancies without allogenic reactivity.

However, challenges remain. CAR-T immunotherapy has been, and immunotherapy with other cell types can be expected to be, limited by the successful expansion of engineered cells in a recipient's body; typically, a large infusion of cells is required. Additionally, loss of persistence of CAR-T cells infused into a subject have been observed, leading to loss of clinical efficacy and potential relapse; other engineered immune effector cells are expected to have similar issues. And to date, CAR-T therapy has been limited to hematologic malignancies due to the tumor microenvironment preventing access by tumor-infiltrating lymphocytes, including engineered cells.

Disclosed herein are therapeutic uses for treating cancer in a subject in need thereof, comprising administering to the subject a population of chimeric antigen receptor (CAR)-bearing immune effector cells and an IL-7 protein.

### SUMMARY OF THE DISCLOSURE

Provided herein is a method for treating a cancer in a subject in need thereof comprising administering to the subject concurrently or sequentially, a) a population of chimeric antigen receptor (CAR)-bearing immune effector cells, and b) an IL-7 protein.

In some embodiments, the IL-7 protein disclosed herein has an amino acid sequence at least about 70%, at least about 80%, at least about 85%, at least about 90%, at least about 95%, at least about 96%, at least about 97%, at least about 98%, at least about 98%, at least about 99%, or about 100% identical to an amino acid sequence selected from SEQ ID NO. 1 (accession no P13232). In certain embodiments, the IL-7 protein is modified.

In some embodiments, the IL-7 protein is a fusion protein. In certain embodiments, the fusion protein comprises an IL-7 protein and a heterologous moiety. In some embodiments, the heterologous moiety is a moiety extending a half-life of the IL-7 protein (“half-life extending moiety”). In some embodiments, the half-life extending moiety is selected from the group consisting of an Fc region of immunoglobulin or a part thereof, albumin, an albumin binding polypeptide, Pro/Ala/Ser (PAS), C-terminal peptide(CTP) of R subunit of human chorionic gonadotropin, polyethylene glycol (PEG), long unstructured hydrophilic sequences of amino acids (XTEN), hydroxyethyl starch(HES), an albumin-binding small molecule, and a combination thereof. In certain embodiments, the half-life extending moiety is an Fc domain.

In some embodiments, the IL-7 protein is a homodimer.

In some embodiments, the IL-7 fusion protein disclosed herein comprises an amino acid sequence at least about 70%, at least about 80%, at least about 85%, at least about 90%, at least about 95%, at least about 96%, at least about 97%, at least about 98%, at least about 98%, at least about 99%, or about 100% identical to an amino acid sequence as set forth in any one of SEQ ID NOs: 21-26.

In some embodiments, the IL-7 protein (e.g., disclosed herein) is to be administered at a weight, based dose between about 20 g/kg and about 600 g/kg or a flat dose of about 0.25 mg to about 9 mg. In certain embodiments, the IL-7 protein is to be administered at a weight-based dose of about 20 μg/kg, about 60 μg/kg, about 120 μg/kg, about 240 μg/kg, about 480 μg/kg, about 600 μg/kg, or about 10 mg/kg or a flat dose of about 0.25 mg, about 1 mg, about 3 mg, about 6 mg, or about 9 mg.

In some embodiments, the IL-7 protein is administered at a dosing interval of at least one week, at least two weeks, at least three weeks, at least four weeks, at least a month, or at least two months. In certain embodiments, the IL-7 protein is administered at a dosing interval of about two weeks or about four weeks. In further embodiments, the IL-7 protein is administered repeatedly. In some embodiments, the IL-7 protein is repeated at least twice, at least three times, at least four times, at least five times, at least six times, at least five times, or more. In certain embodiments, the IL-7 protein is repeated three times.

In some embodiments, the IL-7 protein is administered after the population of chimeric antigen receptor (CAR)-bearing immune effector cells. In certain embodiments, the IL-7 protein is administered when the approximate number of viable immune effector cells in the subject drops below a number needed for efficacy. In further embodiments, the IL-7 protein is administered when a test indicates that the cancer is detected or is relapsing. In some embodiments, the test used to determine whether a cancer is detected or is relapsing is chosen from an imaging test, an ultrasound, a biomarker test, a genetic test, or any combination thereof.

In some embodiments, the IL-7 protein is administered before the population of chimeric antigen receptor (CAR)-bearing immune effector cells. In certain embodiments, the IL-7 protein administered is available at a serum of the subject prior to administering the population of chimeric antigen receptor (CAR)-bearing immune effector cells.

In some embodiments, the IL-7 protein is administered concurrently with the population of chimeric antigen receptor (CAR)-bearing immune effector cells.

In some embodiments, the IL-7 protein achieves one or more of: (i) increased expansion of, (ii) increased persistence of, and/or (iii) increased anti-tumor activity of the population of chimeric antigen receptor (CAR)-bearing immune effector cells.

In some embodiments, the expansion of the population of CAR-bearing immune effector cells is at least about double the expansion that would be achieved without the IL-7 protein. In certain embodiments, the expansion of the population of CAR-bearing immune effector cells is at least about 3×, at least about 4×, at least about 5×, at least about 6×, at least about 7×, at least about 8×, at least about 9×, or at least about 10× the expansion that would be achieved without the IL-7 protein. In further embodiments, the expansion of the population of CAR-bearing immune effector cells is at least about 20×, at least about 30×, at least about 40×, at least about 50×, at least about 60×, at least about 70×, at least about 80×, at least about 90×, or at least about 100× the expansion that would be achieved without the IL-7 protein.

In some embodiments, the population of CAR-bearing immune effector cells persists in the subject in a therapeutically effective quantity for at least twice as long as would be achieved without the IL-7 protein. In certain embodiments, the population of CAR-bearing immune effector cells persists in the subject in a therapeutically effective quantity for at least four times as long as would be achieved without the IL-7 protein.

In some embodiments, the population of CAR-bearing immune effector cells more effectively treat cancer as demonstrated by any of increased survival time, decreased tumor burden, and/or decreased cancer biomarkers (e.g., as would be achieved without the IL-7 protein)

In some embodiments, the CAR-bearing immune effector cells are autologous. In other embodiments, the CAR-bearing immune effector cells are allogenic.

In some embodiments, the CAR-bearing immune effector cells are CAR-T cells, CAR-bearing iNKT cells (iNKT-CAR), or both. In certain embodiments, the CAR-bearing immune effector cells are CAR-T cells. In some embodiments, the CAR targets one or more antigens selected from CD2, CD3ε, CD4, CD5, CD7, CD19, TRAC, BCMA, TCRβ, or combinations thereof.

In some embodiments, the chimeric antigen receptor (CAR)-bearing immune effector cells are genome-edited CAR-T cells. In certain embodiments, the genome-edited CAR-T cells comprise a deletion or modification in one or more antigens selected from CD2, CD3ε, CD4, CD5, CD7, TRAC, TCRβ, or combinations thereof. In some embodiments, the genome-edited CAR-T cells comprise a deletion in CD7. In certain embodiments, the genome-edited CAR-T cells comprise a deletion in CD2. In further embodiments, the genome-edited CAR-T cells additionally comprise a deletion in TRAC. In some embodiments, the genome-edited CAR-T cells are dual or tandem CAR-T cells. In certain embodiments, the genome-edited CAR-T cells are dual CAR-T cells. In other embodiments, the genome-edited CAR-T cells are tandem CAR-T cells.

In some embodiments, the CAR-bearing immune effector cells disclosed herein are CAR-iNKT cells. In certain embodiments, the CAR targets one or more antigens selected from CD2, CD3ε, CD4, CD5, CD7, CD19, TRAC, BCMA, TCRβ, or combinations thereof. In some embodiments, the CAR-iNKT cells comprise a deletion in one or more antigens selected from CD2, CD3ε, CD4, CD5, CD7, TRAC, BCMA, TCRβ, or combinations thereof.

In some embodiments, a cancer that can be treated with the present disclosure comprises a solid tumor. In certain embodiments, the solid tumor is chosen from cervical cancer, pancreatic cancer, ovarian cancer, mesothelioma, squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulvar cancer, thyroid cancer, hepatic carcinoma, anal carcinoma, penile carcinoma head and neck cancer, or any combination thereof.

In some embodiments, the cancer is hematologic malignancy. In certain embodiments, the hematologic malignancy is Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, or any combination thereof. In some embodiments, the hematologic malignancy is a T-cell malignancy. In certain embodiments, the T cell malignancy is T-cell acute lymphoblastic leukemia (T-ALL). In further embodiments, the T cell malignancy is non-Hodgkin's lymphoma. In some embodiments, the hematologic malignancy is multiple myeloma. In certain embodiments, the hematologic malignancy is a B-cell malignancy.

In some embodiments, the IL-7 protein is administered at a dose which reduces the number of chimeric antigen receptor (CAR)-bearing immune effector cells needed to maintain clinical efficacy in the subject. In certain embodiments, the subject is in relapse.

In some embodiments, a dose of the population of chimeric antigen receptor (CAR)-bearing immune effector cells is less than about 100,000 cells per kilogram of the subject's body weight. In certain embodiments, a dose of the population of chimeric antigen receptor (CAR)-bearing immune effector cells is less than about 50,000 cells per kilogram of the subject's body weight. In some embodiments, a dose of the population of chimeric antigen receptor (CAR)-bearing immune effector cells is less than about 10,000 cells per kilogram of the subject's body weight. In further embodiments, a dose of the population of chimeric antigen receptor (CAR)-bearing immune effector cells is less than about 5,000 cells per kilogram of the subject's body weight. In still further embodiments, a dose of the population of chimeric antigen receptor (CAR)-bearing immune effector cells is less than about 2,500 cells per kilogram of the subject's body weight. In certain embodiments, a dose of the population of chimeric antigen receptor (CAR)-bearing immune effector cells is less than about 1,000 cells per kilogram of the subject's body weight.

In some embodiments, the subject is further administered an anti-cancer agent. In certain embodiments, the anti-cancer agent is an immune checkpoint inhibitor. In some embodiments, the immune checkpoint inhibitor is an inhibitor of PD-1, PD-L1, LAG-3, Tim-3, CTLA-4, or any combination thereof. In some embodiments, the immune checkpoint inhibitor is nivolumab, pembrolizumab, ipilimumab, atezolizumab, durvalumab, avelumab, tremelimumab, or any combination thereof.

In some embodiments, the subject is further treated with a lymphocyte depleting agent. In certain embodiments, the lymphocyte depleting agent is administered prior to the population of chimeric antigen receptor (CAR)-bearing immune effector cells. In other embodiments, the lymphocyte depleting agent is administered prior to the IL-7 protein. In further embodiments, the lymphocyte depleting agent is administered prior to the population of chimeric antigen receptor (CAR)-bearing immune effector cells and the IL-7 protein. In certain embodiments, the lymphocyte depleting agent is administered between the population of chimeric antigen receptor (CAR)-bearing immune effector cells and the IL-7 protein.

In some embodiments, the IL-7 protein is administered intravenously, intraperitoneally, intramuscularly, intraarterially, intrathecally, intralymphaticly, intralesionally, intracapsularly, intraorbitally, intracardiacly, intradermally, transtracheally, subcutaneously, subcuticularly, intraarticularly, subcapsularly, subarachnoidly, intraspinally, epidurally or intrasternally.

In some embodiments, about 3 to 100 mg/mL of the IL-7 protein (e.g., disclosed herein) is formulated in about 20 mM sodium citrate, about 5 w/v % sucrose, about 1 to 2 w/v % sorbitol or mannitol, about 0.05 w/v % Tween 80 or poloxamer at a pH of about 5.0.

In some embodiments, the CAR-bearing immune effector cells target BCMA. In certain embodiments, the CAR-bearing immune effector cells express an antibody or antigen-binding portion thereof that specifically binds to BCMA.

In some embodiments, the half-life extending moiety of a fusion protein disclosed herein comprises albumin.

Disclosed herein is a pharmaceutical composition comprising a population of chimeric antigen receptor (CAR)-bearing immune effector cells for use in treating a cancer in combination with an IL-7 protein (e.g., disclosed herein) in a subject in need thereof.

Disclosed herein is a pharmaceutical composition comprising an IL-7 protein for use in treating a cancer in combination with a population of chimeric antigen receptor (CAR)-bearing immune effector cells in a subject in need thereof.

Present disclosure also provides a use of a composition comprising a population of chimeric antigen receptor (CAR)-bearing immune effector cells for the manufacture of a medicament in treating a cancer in combination with an IL-7 protein in a subject in need thereof. Also disclosed herein is a use of a composition comprising an IL-7 protein for the manufacture of a medicament in treating a cancer in combination with an IL-7 protein in a subject in need thereof.

Provided herein is a kit comprising a population of chimeric antigen receptor (CAR)-bearing immune effector cells for use in combination with an IL-7 protein, wherein the kit further comprises instructions according to any one of methods disclosed herein.

Also provided herein is a method of increasing expansion of a population of chimeric antigen receptor (CAR)-bearing immune effector cells in a subject, comprising administering to the subject an interleukin-7 (IL-7) protein in combination with a population of chimeric antigen receptor (CAR)-bearing immune effector cells.

Present disclosure further provides a method of increasing survival of a population of chimeric antigen receptor (CAR)-bearing immune effector cells in a subject, comprising administering to the subject an interleukin-7 (IL-7) protein in combination with a population of chimeric antigen receptor (CAR)-bearing immune effector cells.

Provided herein is a method of improving an anti-tumor activity of a population of chimeric antigen receptor (CAR)-bearing immune effector cells in a subject, comprising administering to the subject an interleukin-7 (IL-7) protein in combination with a population of chimeric antigen receptor (CAR)-bearing immune effector cells.

In some embodiments, a modified IL-7 protein disclosed herein comprises an oligopeptide consisting of 1 to 10 amino acid residues. In certain embodiments, the oligopeptide is selected from the group consisting of methionine, glycine, methionine-methionine, glycine-glycine, methionine-glycine, glycine-methionine, methionine-methionine-methionine, methionine-methionine-glycine, methionine-glycine-methionine, glycine-methionine-methionine, methionine-glycine-glycine, glycine-methionine-glycine, glycine-glycine-methionine, and glycine-glycine-glycine. In further embodiments, the oligopeptide is methionine-glycine-methionine.

In some embodiments, the IL-7 protein is administered less than about one day, less than about two days, less than about three days, less than about four days, less than about five days, less than about six days, less than about one week, less than about two weeks, less than about three weeks, less than about one month, less than about two months, less than about three months, less than about four months, less than about five months, or less than about six months after administering the population of CAR-bearing immune effector cells. In certain embodiments, the IL-7 protein is administered about one day, about two days, about three days, about four days, about five days, about six days, about one week, about two weeks, about three weeks, about one month, about two months, about three months, about four months, about five months, or about six months after administering the population of CAR-bearing immune effector cells.

In some embodiments, the IL-7 protein is administered at least about one day, at least about two days, at least about three days, at least about four days, at least about five days, at least about six days, or at least about one week before administering the population of CAR-bearing immune effector cells.

In some embodiments, an IL-7 protein that can be used in a (i) method of increasing expansion, (ii) method of increasing survival, and/or (iii) method of improving an anti-tumor activity of a population of chimeric antigen receptor (CAR)-bearing immune effector cells disclosed herein is a fusion protein. In certain embodiments, the fusion protein comprises an IL-7 protein and a heterologous moiety. In some embodiments, the heterologous moiety is a moiety extending a half-life of the IL-7 protein (“half-life extending moiety”). In further embodiments, the half-life extending moiety is selected from the group consisting of an Fc region of immunoglobulin or a part thereof, albumin, an albumin binding polypeptide, Pro/Ala/Ser (PAS), C-terminal peptide(CTP) of ˜subunit of human chorionic gonadotropin, polyethylene glycol (PEG), long unstructured hydrophilic sequences of amino acids (XTEN), hydroxyethyl starch(HES), an albumin-binding small molecule, and a combination thereof. In some embodiments, the half-life extending moiety is an Fc domain. In certain embodiments, the IL-7 protein is a homodimer. In certain embodiments, the IL-7 fusion protein comprises an amino acid sequence at least about 70%, at least about 80%, at least about 85%, at least about 90%, at least about 95%, at least about 96%, at least about 97%, at least about 98%, at least about 98%, at least about 99%, or about 100% identical to an amino acid sequence as set forth in any one of SEQ ID NOs: 21-26.

In some embodiments, the CAR-bearing immune effector cells that can be used in a (i) method of increasing expansion, (ii) method of increasing survival, and/or (iii) method of improving an anti-tumor activity of a population of chimeric antigen receptor (CAR)-bearing immune effector cells disclosed herein are allogenic. In certain embodiments, the CAR-bearing immune effector cells are iNKT-CAR cells. In some embodiments, the CAR-bearing immune effector cells specifically bind to one or more antigens in Tables 3, 4, and 5.

### BRIEF DESCRIPTION OF THE DRAWINGS

The patent or application file contains at least one photograph executed in color. Copies of this patent or patent application publication with color photograph(s) will be provided by the Office upon request and payment of the necessary fee.

FIG. 1A shows a schematic diagram of a chimeric antigen receptor (top) that expresses an anti-CD19 scFv and generation of universal chimeric antigen receptor (CAR) T cells (UCART19) (bottom). FIG. 1B shows an experimental design of a combination therapy of UCART19 with NT-I7.

FIG. 2A-FIG. 2H show the comparison data resulting from the combination therapy shown in FIG. 1B: (i) no treatment (no tx), (ii) NT-I7 only, (iii) UCART19 only, and (iv) UCART19 and NT-I7 combination. FIG. 2A provides a comparison of the kinetics of tumor burden (i.e., number of GFP+ Ramos cells) in the animals from the different treatment groups. FIG. 2B provides the survival curve for the animals from the different treatment groups. FIG. 2C provides a comparison of tumor burden (as measured by bioluminescence assay) in the animals from the different treatment groups. FIG. 2D provides FACS analysis showing the frequency of GFP+ Ramos (i.e., tumor cells) in the blood of animals from the different treatment groups at week 3. FIG. 2E provides a comparison of the number of tumor cells in the blood of animals from the different treatment groups at weeks 2, 3, 4, 5, and 6 post administration of the UCART19 cells. FIG. 2F provides a FACS analysis of the frequency of UCART19 (i.e., CD34+CD45+ GFP-CD4+) cells in the blood of a representative animal from groups that received UCART19 only (top row) or that received UCART19 in combination with NT-I7 (bottom row). FIG. 2G shows the change in the number of UCART19 cells over a course of about 6 weeks in the blood of animals treated with UCART19 alone (circle) or in combination with NT-I7 (box). FIG. 2H provides a comparison of the frequency of UCART19 cells in the blood of animals treated with UCART19 alone (square) or in combination with NT-I7 (circle) at week 3 post UCART administration. The frequency of UCART19 cells is shown both as a percentage of total CD45+ GFP−CD34+ cells (left graph) and as absolute number (right graph). In FIG. 2H, the cells were further classified as (i) CD4+, (ii) CD8+, (iii)_ CD4− CD8− (“DN”), and (iv) CD4+CD8+(DP). The above data shows that UCART19 with NT-I7 administration kill Ramos and indefinitely prolong survival. Ramos GFP-CBR NSG mice treated with UCART19 and NTI7 show massive expansion of circulating huCD45+ GFP-CD34+ UCART19 cells compared to mice receiving UCART19 alone.

FIG. 2I shows NT-I7 rapidly expanding CD4+ UCART19 cells at week 3 post UCART19 administration.

FIG. 3A shows the construct diagram of a chimeric antigen receptor expressing an anti-CD2 scFv (top) and generation of universal chimeric antigen receptor (CAR) T cells (UCART2) (bottom). FIG. 3B shows an experimental design of a combination therapy of UCART2 and NT-I7 for the treatment of T cell hematologic malignancies.

FIG. 3C-FIG. 3D show that the UCART2 and NT-I7 combination reduces tumor burden. FIG. 3C provides the survival curve, and FIG. 3D shows the tumor burden over a course of 28 days. The different treatment groups included the following: (i) no treatment (i.e., tumor only); (ii) NT-I7 alone; (iii) UCART19 alone; (iv) UCART19 and NT-I7; (v) UCART2 alone; and (vi) UCART+NT-I7.

FIG. 4A-FIG. 4B show that NT-I7 promotes rapid UCART19 expansion in all hematopoietic cells (Spleen, Blood, Marrow). FIG. 4A provides a schematic of the experimental design. FIG. 4B provides a comparison of the number of UCART19 cells in the blood, femur, and spleen of animals that received either UCART19 cells alone (circle) or UCART19 cells in combination with NT-I7 (rectangle). The top row shows the data at 1 week post administration. The bottom row shows the data at 2 weeks post administration.

FIGS. 5A, 5B, 5C, and 5D show the anti-tumor effects of B-Cell Maturation Antigen (BCMA)-specific CAR iNKT and CD19-specific CAR iNKT cells, alone or in combination with NT-I7, in a mouse model of multiple myeloma. FIG. 5A provides a schematic of the experimental design. FIG. 5B provides a comparison of the survival data in tumor mice treated with one of the following regimens: (i) CD19 CAR-T cells+vehicle control “(1)”; (ii) CD19 CAR-T cells+NT-I7 “(2)”; (iii) BCMA CAR-T cells+vehicle “(3)”; and (iv) BCMA CAR-T cells+NT-I7 “(4)”. FIGS. 5C and 5D provide a comparison of tumor burden (as measured by bioluminescence assay) in tumor mice treated with BCMA CAR T cells alone or in combination with NT-I7. The treatment groups are the same as in FIG. 5A. In FIGS. 5B, 5C, and 5D, as controls, some of the animals were left untreated or treated with UCART19, alone or in combination with NT-I7.

FIGS. 6A, 6B, 6C, and 6D show the anti-tumor effects of C-type lectin-like molecule-1 (CLL-1)-specific CAR T cells and NT-I7 combination in a mouse model of acute myeloid leukemia. FIG. 6A provides a schematic of the experimental design. FIG. 6B provides a comparison of T cell numbers in the peripheral blood of animals treated with CAR T cells alone (closed circle) or in combination with NT-I7 (inverted open triangle). FIG. 6C provides a comparison of tumor growth (as determined by bioluminescence assay) and FIG. 6D provides survival curves of animals from the different treatment groups. In FIGS. 6C and 6D, the treatment groups included the following: (i) untreated (closed circle), (ii) NT-I7 alone (open circle), (iii) CAR T cells alone (triangle), and (iv) both CAR T cells and NT-I7 (inverted triangle).

FIGS. 7A, 7B, and 7C show the anti-tumor response after re-challenge of tumor free mice with MM.1S-CG tumor cells. The tumor free mice (n=7) were those animals from FIGS. 5A-5D that received the combination of BCMA-specific CAR iNKT cells and NT-I7, and showed no tumor burden by bioluminescence assay on day 221 post tumor induction. Some of the animals received vehicle (n=3) while other received a second course of NT-I7 (n=4) with the tumor re-challenge. As a positive control, naïve mice (i.e., did not previously receive any treatment) were treated with tumor and vehicle alone. FIG. 7A provide a comparison of tumor burden (as determined by bioluminescence assay). FIG. 7B shows the same data as in FIG. 7A but graphically. The treatment groups included: (i) naïve mice that received tumor and vehicle alone (i.e., positive control) “black lines”; (ii) tumor-free mice that received vehicle “light gray lines”; and (iii) tumor-free mice that received second course of NT-I7 “dark gray lines.” For each of the treatment groups, each line represents an individual animal. FIG. 7C provide a comparison of the number CAR iNKT cells in the blood for the different treatment groups. The treatment groups are the same as in FIG. 7B. In both the positive control group and the tumor-free mice that received vehicle group, the number of CAR iNKT cells detected were negligible (i.e., lines run along the x-axis).

### DETAILED DESCRIPTION OF THE INVENTION

### I. Methods of the Disclosure

Disclosed herein is a method for treating a cancer in a subject in need thereof comprising administering to the subject concurrently or sequentially,
a. a population of chimeric antigen receptor (CAR)-bearing immune effector cells, and
b. an IL-7 protein.

Also disclosed herein is a method of increasing expansion of a population of CAR-bearing immune effector cells in a subject, comprising administering to the subject an IL-7 protein (e.g., those disclosed herein) in combination with a population of CAR-bearing immune effector cells (e.g., those disclosed herein). In some embodiments, expansion of the population of CAR-bearing immune effector cells is increased by at least about 5%, at least about 10%, at least about 20%, at least about 30%, at least about 40%, at least about 50%, at least about 60%, at least about 70%, at least about 80%, at least about 90%, at least about 100% or more, compared to a reference (e.g., expansion in the absence of administration of an IL-7 protein disclosed herein). Where the IL-7 protein being administered is modified (e.g., IL-7 fusion proteins disclosed herein), in some embodiments, the reference is the expansion of the population of CAR-bearing immune effector cells with wild-type IL-7 protein administration.

Present disclosure also provides a method of increasing survival of a population of CAR-bearing immune effector cells in a subject, comprising administering to the subject an IL-7 protein (e.g., those disclosed herein) in combination with a population of CAR-bearing immune effector cells (e.g., those disclosed herein). In some embodiments, survival of the population of CAR-bearing immune effector cells is increased by at least about 5%, at least about 10%, at least about 20%, at least about 30%, at least about 40%, at least about 50%, at least about 60%, at least about 70%, at least about 80%, at least about 90%, at least about 100% or more, compared to a reference (e.g., survival in the absence of administration of an IL-7 protein disclosed herein). Where the IL-7 protein being administered is modified (e.g., IL-7 fusion proteins disclosed herein), in some embodiments, the reference is the survival of the population of CAR-bearing immune effector cells with wild-type IL-7 protein administration.

Also provided herein is a method of improving an anti-tumor activity of a population of CAR-bearing immune effector cells in a subject, comprising administering to the subject an IL-7 protein (e.g., those disclosed herein) in combination with a population of CAR-bearing immune effector cells (e.g., those disclosed herein). In some embodiments, the anti-tumor activity of the population of CAR-bearing immune effector cells is increased by at least about 5%, at least about 10%, at least about 20%, at least about 30%, at least about 40%, at least about 50%, at least about 60%, at least about 70%, at least about 80%, at least about 90%, at least about 100% or more, compared to a reference (e.g., anti-tumor activity in the absence of administration of an IL-7 protein disclosed herein). Where the IL-7 protein being administered is modified (e.g., IL-7 fusion proteins disclosed herein), in some embodiments, the reference is the anti-tumor activity of the population of CAR-bearing immune effector cells with wild-type IL-7 protein administration.

In certain embodiments, the population of chimeric antigen receptor (CAR)-bearing immune effector cells and the an IL-7 protein are administered concurrently. In some embodiments, when the CAR-bearing immune effectors cells and the IL-7 protein are administered concurrently, they are administered separately (i.e., not as a single unit, e.g., both the CAR and IL-7 are not expressed by a single cell). In certain embodiments, the population of chimeric antigen receptor (CAR)-bearing immune effector cells and the an IL-7 protein are administered sequentially.

In certain embodiments, the IL-7 protein has an amino acid sequence at least about 70%, at least about 80%, at least about 85%, at least about 90%, at least about 95%, at least about 96%, at least about 97%, at least about 98%, at least about 98%, at least about 99%, or about 100% identical to an amino acid sequence selected from SEQ ID NO. 1 (accession no P13232). Additional examples of IL-7 proteins that can be used with the present methods are described elsewhere in this present disclosure.

In certain embodiments, the IL-7 protein is modified.

In certain embodiments, the IL-7 protein is an IL-7 fusion protein.

In certain embodiments, the fusion protein comprises an IL-7 protein and a heterologous moiety.

In certain embodiments, the heterologous moiety is a moiety extending a half-life of the IL-7 protein (“half-life extending moiety”). In some embodiments, the half-life of the IL-7 protein is extended by at least about 5%, at least about 10%, at least about 15%, at least about 20%, at least about 30%, at least about 40%, at least about 50%, at least about 60%, at least about 70%, at least about 80%, at least about 90, at least about 100% or more, compared to a reference IL-7 protein (e.g., the same IL-7 protein that is not conjugated to a half-life extending moiety).

In certain embodiments, the half-life extending moiety is selected from the group consisting of an Fc region of immunoglobulin or a part thereof, albumin, an albumin binding polypeptide, Pro/Ala/Ser (PAS), C-terminal peptide(CTP) of˜subunit of human chorionic gonadotropin, polyethylene glycol (PEG), long unstructured hydrophilic sequences of amino acids (XTEN), hydroxyethyl starch(HES), an albumin-binding small molecule, and a combination thereof.

In certain embodiments, the half-life extending moiety is an Fc domain.

In certain embodiments, the heterologous moiety is a moiety that improves one or more properties of an IL-7 protein.

In certain embodiments, the IL-7 protein is a homodimer,

In certain embodiments, the IL-7 fusion protein (e.g., comprises an IL-7 protein and a heterologous moiety disclosed herein) comprises an amino acid sequence that is at least about 70%, at least about 80%, at least about 85%, at least about 90%, at least about 95%, at least about 96%, at least about 97%, at least about 98%, at least about 98%, at least about 99%, or about 100% identical to an amino acid sequence as set forth in any one of SEQ ID NOs: 21-26.

In certain embodiments, the IL-7 protein is to be administered at a weight, based dose between about 20 g/kg and about 600 g/kg (or between 20 g/kg and about 2000 g/kg) or a flat dose of about 0.25 mg to about 9 mg.

In certain embodiments, the IL-7 protein is to be administered at a weight-based dose of between about 20 μg/kg to about 10 mg/kg. In some embodiments, the IL-7 protein of the present disclosure is administered to a subject at a weight-based dose of about 20 μg/kg, about 60 μg/kg, about 120 μg/kg, about 240 μg/kg, about 480 μg/kg, about 600 μg/kg, about 2,000 μg/kg, or about 10 mg/kg. In some embodiments, the IL-7 protein disclosed herein is administered at a flat dose of about 0.25 mg to about 9 mg. In certain embodiments, the IL-7 protein is administered at a flat dose of about 0.25 mg, about 1 mg, about 3 mg, about 6 mg, or about 9 mg.

In certain embodiments, the IL-7 protein is administered at a dosing interval of at least one week, at least two weeks, at least three weeks, at least four weeks, at least a month, or at least two months.

In certain embodiments, the IL-7 protein is administered at a dosing interval of about two weeks or about four weeks.

In certain embodiments, the IL-7 protein is administered repeatedly.

In certain embodiments, the IL-7 protein is repeated at least twice, at least three times, at least four times, at least five times, at least six times, at least five times, or more.

In certain embodiments, the IL-7 protein is repeated three times at a dosing interval of greater than one week.

In certain embodiments, the IL-7 protein is administered after the population of chimeric antigen receptor (CAR)-bearing immune effector cells is administered to the subject. In some embodiments, the IL-7 protein is administered less than about one hour, less than about two hours, less than about three hours, less than about four hours, less than about five hours, less than about six hours, less than about twelve hours, less than about one day, less than about two days, less than about three days, less than about four days, less than about five days, less than about six days, less than about one week, less than about two weeks, less than about three weeks, less than about one month, less than about two months, less than about three months, less than about four months, less than about five months, or less than about six months after administering the population of CAR-bearing immune effector cells. In certain embodiments, the IL-7 protein is administered about one hour, about two hours, about three hours, about four hours, about five hours, about six hours, about twelve hours, about one day, about two days, about three days, about four days, about five days, about six days, about one week, about two weeks, about three weeks, about one month, about two months, about three months, about four months, about five months, or about six months after administering the population of CAR-bearing immune effector cells. In some embodiments, an IL-7 protein disclosed herein is administered to the subject about one day after the administration of the population of CAR-bearing immune effector cells.

In certain embodiments, the IL-7 protein is administered when the approximate number of viable immune effector cells in the subject drops below a number needed for efficacy.

In certain embodiments, the IL-7 protein is administered when a test indicates that the cancer is detected or is relapsing. Any tests known in the art can be used to determine whether a cancer is detected or is relapsing. In certain embodiments, the test is chosen from an imaging test, an ultrasound, a biomarker test, a genetic test, a flow cytometry test (e.g., as that described in worldwideweb.mayocliniclabs.com/test-catalog/Performance/19499), or any combination thereof.

In certain embodiments, the IL-7 protein is administered before the population of chimeric antigen receptor (CAR)-bearing immune effector cells. In some embodiments, the IL-7 protein is administered at least about one hour, at least about two hours, at least about three hours, at least about four hours, at least about five hours, at least about six hours, at least about twelve hours, at least about one day, at least about two days, at least about three days, at least about four days, at least about five days, at least about six days, or at least about one week before administering the population of CAR-bearing immune effector cells.

In certain embodiments, the IL-7 protein administered is available at a serum of the subject prior to administering the population of chimeric antigen receptor (CAR)-bearing immune effector cells.

In certain embodiments, the IL-7 protein is administered concurrently with the population of chimeric antigen receptor (CAR)-bearing immune effector cells. In further embodiments, the IL-7 protein is administered prior to, concurrently, and/or after administering the CAR-bearing immune effector cells to a subject.

In certain embodiments, the IL-7 protein is not a wild-type IL-7 protein and has been modified (e.g., an IL-7 fusion protein disclosed herein). The modified IL-7 protein can improve one or more properties of CAR-bearing immune effector cells. Non-limiting examples of such improved properties include one or more of:
(i) increased expansion of,
(ii) increased persistence of, and/or
(iii) increased anti-tumor activity (e.g., ability to target and kill a tumor cell) of the population of chimeric antigen receptor (CAR)-bearing immune effector cells.

In certain embodiments, the expansion of the population of CAR-bearing immune effector cells in a subject after administering the IL-7 protein (e.g., those disclosed herein) is at least about double the expansion that would be achieved without the IL-7 protein.

In certain embodiments, the expansion of the population of CAR-bearing immune effector cells in a subject after administering the IL-7 protein of the present disclosure is at least about 3×, at least about 4×, at least about 5×, at least about 6×, at least about 7×, at least about 8×, at least about 9×, or at least about 10× the expansion that would be achieved without the IL-7 protein.

In certain embodiments, the expansion of the population of CAR-bearing immune effector cells is at least about 20×, at least about 30×, at least about 40×, at least about 50×, at least about 60×, at least about 70×, at least about 80×, at least about 90×, or at least about 100× the expansion that would be achieved without the IL-7 protein.

In certain embodiments, the number of CAR-bearing immune effectors cells in the subject is increased by at least about 5%, at least about 10%, at least about 20%, at least about 30%, at least about 40%, at least about 50%, at least about 60%, at least about 70%, at least about 80%, at least about 90%, at least about 100% or more, compared to a reference subject (e.g., received no IL-7 protein or received wild-type IL-7 protein).

In certain embodiments, the population of CAR-bearing immune effector cells persists in the subject treated with an IL-7 protein disclosed herein in a therapeutically effective quantity for at least twice as long as would be achieved without the IL-7 protein.

In certain embodiments, the population of CAR-bearing immune effector cells persists in the subject treated with an IL-7 protein in a therapeutically effective quantity for at least four times as long as would be achieved without the IL-7 protein.

In certain embodiments, the survival (i.e., persistence) of the CAR-bearing immune effector cells in the subject is increased by at least about 5%, at least about 10%, at least about 20%, at least about 30%, at least about 40%, at least about 50%, at least about 60%, at least about 70%, at least about 80%, at least about 90%, at least about 100% or more, compared to a reference subject (e.g., received no IL-7 protein or received wild-type IL-7 protein).

In some embodiments, an IL-7 protein of the present disclosure can increase the killing potential of the CAR-bearing immune effector cells in the subject. In some embodiments, the killing potential of a CAR-bearing immune effector cell is increased by at least about 5%, at least about 10%, at least about 20%, at least about 30%, at least about 40%, at least about 50%, at least about 60%, at least about 70%, at least about 80%, at least about 90%, at least about 100% or more, compared to a reference subject (e.g., received no IL-7 protein or received wild-type IL-7 protein).

In certain embodiments, the population of CAR-bearing immune effector cells, when administered in combination with an IL-7 protein disclosed herein, can more effectively treat cancer as demonstrated by any of increased survival time, decreased tumor burden, and/or decreased cancer biomarkers.

In some embodiments, the survival time of a subject treated with the combination of CAR-bearing immune effectors cells and IL-7 protein (e.g., those disclosed herein) is increased by at least about 5%, at least about 10%, at least about 20%, at least about 30%, at least about 40%, at least about 50%, at least about 60%, at least about 70%, at least about 80%, at least about 90%, at least about 100% or more, compared to a reference subject (e.g., received no IL-7 protein or received wild-type IL-7 protein).

In some embodiments, the tumor burden in a subject treated with the combination of CAR-bearing immune effectors cells and IL-7 protein (e.g., those disclosed herein) is decreased by at least about 5%, at least about 10%, at least about 20%, at least about 30%, at least about 40%, at least about 50%, at least about 60%, at least about 70%, at least about 80%, at least about 90%, or at least about 100%, compared to a reference subject (e.g., received no IL-7 protein or received wild-type IL-7 protein).

In some embodiments, the expression of one or more cancer biomarkers is decreased in a subject with the combination of CAR-bearing immune effectors cells and IL-7 protein (e.g., those disclosed herein) is decreased by at least about 5%, at least about 10%, at least about 20%, at least about 30%, at least about 40%, at least about 50%, at least about 60%, at least about 70%, at least about 80%, at least about 90%, or at least about 100%, compared to a reference subject (e.g., received no IL-7 protein or received wild-type IL-7 protein).

In certain embodiments, the CAR-bearing immune effector cells are autologous. As used herein, the term “autologous” refers to material derived from the same individual to whom it is later to be re-introduced into the individual.

In certain embodiments, the CAR-bearing immune effector cells are allogenic. As used herein, the term “allogenic” refers to material derived from a different subject of the same species as the individual to whom the material is introduced. Two or more individuals are said to be allogeneic to one another when the genes at one or more loci are not identical. In some aspects, allogeneic material from individuals of the same species can be sufficiently unlike genetically to interact antigenically.

In certain embodiments, the CAR-bearing immune effector cells are CAR-T cells, CAR-bearing invariant natural killer T (iNKT) cells (iNKT-CAR), or both.

In certain embodiments, the chimeric antigen receptor of the CAR-bearing immune effector cell targets (specifically binds) one or more antigens expressed on a tumor cell, such as a malignant B cell, a malignant T cell, or malignant plasma cell.

In certain embodiments, the CAR targets one or more antigens selected from CD2, CD3ε, CD4, CD5, CD7, CD19, TRAC, TCRβ, BCMA, CLL-1, CS1, CD38, CD19, the extracellular portion of the APRIL protein, or combination thereof

In certain embodiments, the antigen is selected from BCMA, CLL-1, CS1, CD38, CD19, or combinations thereof.

In certain embodiments, the chimeric antigen receptor expresses the extracellular portion of the APRIL protein, the ligand for BCMA and TACI, effectively co-targeting both BCMA and TACI.

In certain embodiments, the CAR targets one or more antigens selected from CD2, CD3ε, CD4, CD5, CD7, CD19, TRAC, TCRβ, or combinations thereof.

In certain embodiments, the CAR-bearing immune effector cells are genome-edited.

In certain embodiments, the CAR-bearing immune effector cells are CAR-T cells.

In certain embodiments, the CAR-T cells comprise at least one CAR, targeting one or more antigens, and are deficient in an antigen to which the CAR specifically binds.

In certain embodiments, the genome-edited CAR-T cells comprise a deletion or modification in one or more antigens selected from CD2, CD3ε, CD4, CD5, CD7, TRAC, TCRβ, or combinations thereof.

In certain embodiments, the genome-edited CAR-T cells comprise a deletion in CD7.

In certain embodiments, the genome-edited CAR-T cells comprise a deletion in CD2.

In certain embodiments, the genome-edited CAR-T cells additionally comprise a deletion in one of TRAC, TCRβ, and CD3ε.

In certain embodiments, the genome-edited CAR-T cells additionally comprise a deletion in TRAC.

In certain embodiments, the genome-edited CAR-T cells are dual or tandem CAR-T cells.

In certain embodiments, the genome-edited CAR-T cells are dual CAR-T cells.

In certain embodiments, the genome-edited CAR-T cells are tandem CAR-T cells.

In certain embodiments, the CAR-bearing immune effector cells are CAR-iNKT cells.

In certain embodiments, in the CAR-bearing immune effector cells comprise a deletion in one or more antigens selected from CD2, CD3ε, CD4, CD5, CD7, TRAC, TCRβ, or combinations thereof.

In certain embodiments, the chimeric antigen receptor CAR-bearing immune effector cells each further comprise a suicide gene.

In certain embodiments, endogenous T cell receptor mediated signaling is blocked in the CAR-bearing immune effector cells.

In certain embodiments, the chimeric antigen receptor CAR-bearing immune effector cells do not induce alloreactivity or graft-versus-host disease.

In certain embodiments, the chimeric antigen receptor CAR-bearing immune effector cells do not induce fratricide.

In certain embodiments, the CAR-bearing immune effector cells comprise tandem CAR-T cells or tandem iNKT-CAR cells, or both.

In certain embodiments, the CAR-bearing immune effector cells comprise dual CAR-T cells or dual iNKT-CAR cells, or both.

Additional details relating to CAR-bearing immune effector cells that are useful for the present methods are disclosed elsewhere in the present disclosure.

In certain embodiments, the cancer comprises a solid tumor.

In certain embodiments, the solid tumor is chosen from cervical cancer, pancreatic cancer, ovarian cancer, mesothelioma, squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulvar cancer, thyroid cancer, hepatic carcinoma, anal carcinoma, penile carcinoma head and neck cancer, or any combination thereof.

In certain embodiments, the cancer is hematologic malignancy.

In certain embodiments, the hematologic malignancy is Acute Childhood Lymphoblastic Leukemia, Acute Lymphoblastic Leukemia, Acute Lymphocytic Leukemia, Acute Myeloid Leukemia (AML), Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, or any combination thereof.

In certain embodiments, the hematologic malignancy is a T-cell malignancy.

In certain embodiments, the T cell malignancy is T-cell acute lymphoblastic leukemia (T-ALL).

In certain embodiments, the T cell malignancy is non-Hodgkin's lymphoma.

In certain embodiments, the hematologic malignancy is multiple myeloma.

In certain embodiments, the hematologic malignancy is a B-cell malignancy.

In certain embodiments, the IL-7 protein is administered at a dose which reduces the number of chimeric antigen receptor (CAR)-bearing immune effector cells needed to maintain clinical efficacy in the subject.

In certain embodiments, the subject is in relapse. As used herein, the term “relapse” refers to the return of a cancer disease or the signs and symptoms of a cancer disease after a period of improvement in which no cancer could be detected.

In certain embodiments, the dose of the population of chimeric antigen receptor (CAR)-bearing immune effector cells is less than about 100,000 cells per kilogram of the subject's body weight.

In certain embodiments, the dose of the population of chimeric antigen receptor (CAR)-bearing immune effector cells is less than about 50,000 cells per kilogram of the subject's body weight.

In certain embodiments, the dose of the population of chimeric antigen receptor (CAR)-bearing immune effector cells is less than about 10,000 cells per kilogram of the subject's body weight.

In certain embodiments, the dose of the population of chimeric antigen receptor (CAR)-bearing immune effector cells is less than about 5,000 cells per kilogram of the subject's body weight.

In certain embodiments, the dose of the population of chimeric antigen receptor (CAR)-bearing immune effector cells is less than about 2,500 cells per kilogram of the subject's body weight.

In certain embodiments, the dose of the population of chimeric antigen receptor (CAR)-bearing immune effector cells is less than about 1,000 cells per kilogram of the subject's body weight.

In some embodiments, the dose of the population of CAR-bearing immune effector cells is reduced when administered in combination with an IL-7 protein of the present disclosure. In certain embodiments, the dose of the population of CAR-bearing immune effector cells is reduced by at least about 5%, at least about 10%, at least about 20%, at least about 30%, at least about 40%, at least about 50%, at least about 60%, at least about 70%, at least about 80%, at least about 90%, at least about 100% or more, compared to a reference dose (e.g., corresponding dose when administered without IL-7 protein or dose when administered with wild-type IL-7 protein).

In certain embodiments, the subject is further administered an anti-cancer agent.

In certain embodiments, the anti-cancer agent is an immune checkpoint inhibitor.

In certain embodiments, the immune checkpoint inhibitor is an inhibitor of PD-1, PD-L1, LAG-3, Tim-3, CTLA-4, or any combination thereof.

In certain embodiments, the immune checkpoint inhibitor is nivolumab (OPDIVO®), pembrolizumab (KEYTRUDA®), ipilimumab (YERVOY®), atezolizumab (TECENTRIQ®), durvalumab (IMFINZI®), avelumab (BAVENCIO®), tremelimumab, or any combination thereof.

In certain embodiments, the subject is further treated with a lymphocyte depleting agent. Non-limiting examples of lymphocyte depleting agents include antibodies (e.g., THYMOGLOBULIN®, ATGAM®, CAMPATH®) and chemotherapy agents (e.g., fludarabine (FLUDARA®) and cyclophosphamide (CYTOXAN®).

In some embodiments, the subject is further treated with a kinase inhibitor (e.g., dasatinib (SPRYCEL®)). In certain embodiments, the kinase inhibitor can be used to reversibly block CAR-T cell function (e.g., to mitigate cytokine release syndrome).

In certain embodiments, the lymphocyte depleting agent is administered prior to the population of chimeric antigen receptor (CAR)-bearing immune effector cells.

In certain embodiments, the lymphocyte depleting agent is administered prior to the IL-7 protein.

In certain embodiments, the lymphocyte depleting agent is administered prior to the population of chimeric antigen receptor (CAR)-bearing immune effector cells and the IL-7 protein.

In certain embodiments, the lymphocyte depleting agent is administered between the population of chimeric antigen receptor (CAR)-bearing immune effector cells and the IL-7 protein.

In certain embodiments, the IL-7 protein is administered intravenously, intraperitoneally, intramuscularly, intraarterially, intrathecally, intralymphaticly, intralesionally, intracapsularly, intraorbitally, intracardiacly, intradermally, transtracheally, subcutaneously, subcuticularly, intraarticularly, subcapsularly, subarachnoidly, intraspinally, epidurally or intrasternally.

In certain embodiments, about 3 to 100 mg/mL of the IL-7 protein is formulated in about 20 mM sodium citrate, about 5 w/v % sucrose, about 1 to 2 w/v % sorbitol or mannitol, about 0.05 w/v % Tween 80 or poloxamer at a pH of about 5.0.

Also provided herein is a pharmaceutical composition comprising a population of chimeric antigen receptor (CAR)-bearing immune effector cells for use in treating a cancer in combination with an IL-7 protein (e.g., those disclosed herein) in a subject in need thereof.

Also provided is a pharmaceutical composition comprising an IL-7 protein for use in treating a cancer in combination with a population of chimeric antigen receptor (CAR)-bearing immune effector cells in a subject in need thereof.

Also provided is the use of a composition comprising a population of chimeric antigen receptor (CAR)-bearing immune effector cells for the manufacture of a medicament in treating a cancer in combination with an IL-7 protein in a subject in need thereof.

Also provided is the use of a composition comprising an IL-7 protein for the manufacture of a medicament in treating a cancer in combination with an IL-7 protein in a subject in need thereof.

Also provided is a kit comprising a population of chimeric antigen receptor (CAR)-bearing immune effector cells for use in combination with an IL-7 protein, wherein the kit further comprises instructions according to any one of the methods disclosed herein.

The present methods are directed to a combination therapy of a population of T cells, e.g., CAR-bearing immune effector cells, and an IL-7 protein to treat a disease. In some embodiments, the disease can be a hyperproliferative disease or disorder, e.g., a cancer. The cancer can be solid tumor or hematological malignancy. The solid organ malignancy can be cervical cancer, pancreatic cancer, ovarian cancer, mesothelioma, and lung cancer. The hematologic malignancy can be multiple myeloma or a T-cell malignancy. The T-cell malignancy can be T-cell acute lymphoblastic leukemia (T-ALL) or non-Hodgkin's lymphoma.

By “hyperproliferative disease or disorder” is meant all neoplastic cell growth and proliferation, whether malignant or benign, including all transformed cells and tissues and all cancerous cells and tissues. Hyperproliferative diseases or disorders include, but are not limited to, precancerous lesions, abnormal cell growths, benign tumors, malignant tumors, and “cancer.”

Additional examples of hyperproliferative diseases, disorders, and/or conditions include, but are not limited to neoplasms, whether benign or malignant, located in the: prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital tract.

As used herein, the terms “tumor” or “tumor tissue” refer to an abnormal mass of tissue that results from excessive cell division. A tumor or tumor tissue comprises “tumor cells” which are neoplastic cells with abnormal growth properties and no useful bodily function. Tumors, tumor tissue and tumor cells can be benign or malignant. A tumor or tumor tissue can also comprise “tumor-associated non-tumor cells”, e.g., vascular cells which form blood vessels to supply the tumor or tumor tissue. Non-tumor cells can be induced to replicate and develop by tumor cells, for example, the induction of angiogenesis in a tumor or tumor tissue.

As used herein, the term “malignancy” refers to a non-benign tumor or a cancer. As used herein, the term “cancer” connotes a type of hyperproliferative disease which includes a malignancy characterized by deregulated or uncontrolled cell growth. Examples of cancer include, but are not limited to, carcinoma, lymphoma, blastoma, sarcoma, and leukemia or lymphoid malignancies. More particular examples of such cancers are noted below and include: squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulvar cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia (AML), Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

The method of the present disclosure can be used to treat premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, Basic Pathology, 2d Ed., W. B. Saunders Co., Philadelphia, pp. 68-79 (1976)

In some embodiments, the present methods further comprise administering an anti-cancer agent, e.g., an immune checkpoint inhibitor, e.g., PD-1, PD-L1, LAG-3, Tim-3, CTLA-4, or any combination thereof. In some embodiments, the checkpoint inhibitor is nivolumab (OPDIVO®), pembrolizumab (KEYTRUDA®), ipilimumab (YERVOY®), atezolizumab (TECENTRIQ®), durvalumab (IMFINZI®), avelumab (BAVENCIO®), tremelimumab, or any combination thereof.

In other embodiments, the present methods comprise further administering to the subject a lymphocyte depleting agent in combination with the IL-7 protein and the CAR-bearing immune effector cells. In some embodiments, the lymphocyte depleting agent is administered prior to the population of chimeric antigen receptor (CAR)-bearing immune effector cells. In other embodiments, the lymphocyte depleting agent is administered prior to the IL-7 protein. In some embodiments, the lymphocyte depleting agent is administered prior to the population of chimeric antigen receptor (CAR)-bearing immune effector cells and the IL-7 protein. In other embodiments, the lymphocyte depleting agent is administered between the population of chimeric antigen receptor (CAR)-bearing immune effector cells and the IL-7 protein.

In other embodiments, the present disclosure includes a kit comprising a population of chimeric antigen receptor (CAR)-bearing immune effector cells for use in combination with an IL-7 protein, wherein the kit further comprises instructions according to any methods disclosed herein.

### II. Interleukin-7 Protein

Disclosed herein are combinations and uses in combination of CAR-bearing immune-effector cells, such as CAR-T and/or CAR-iNKT cells, with native and/or modified interleukin-7 (IL-7) protein.

The IL-7 protein useful for the present uses can be wild-type IL-7 or modified IL-7 (e.g., IL-7 variant, IL-7 functional fragment, IL-7 derivative, or any combination thereof, e.g., fusion protein, chimeric protein, etc.) as long as the IL-7 protein contains one or more biological activities of IL-7, e.g., capable of binding to IL-7R, e.g., inducing early T-cell development, promoting T-cell homeostasis. See ElKassar and Gress. J Immunotoxicol. 2010 March; 7(1): 1-7. In some embodiments, the IL-7 protein is a modified IL-7 protein.

IL-7 binds to its receptor which is composed of the two chains IL-7Rα (CD127), shared with the thymic stromal lymphopoietin (TSLP) (Ziegler and Liu, 2006), and the common γ chain (CD132) for IL-2, IL-15, IL-9 and IL-21. Whereas ye is expressed by most hematopoietic cells, IL-7Rα is nearly exclusively expressed on lymphoid cells. After binding to its receptor, IL-7 signals through two different pathways: Jak-Stat (Janus kinase-Signal transducer and activator of transcription) and PI3K/Akt responsible for differentiation and survival, respectively. The absence of IL-7 signaling is responsible for a reduced thymic cellularity as observed in mice that have received an anti-IL-7 neutralizing monoclonal antibody (MAb); Grabstein et al., 1993), in IL-7−/− (von Freeden-Jeffry et al., 1995), IL-7Rα−/− (Peschon et al., 1994; Maki et al., 1996), γc−/− (Malissen et al., 1997), and Jak3−/− mice (Park et al., 1995). In the absence of IL-7 signaling, mice lack T-, B-, and NK-T cells. IL-7α−/− mice (Peschon et al., 1994) have a similar but more severe phenotype than IL-7−/− mice (von Freeden-Jeffry et al., 1995), possibly because TSLP signaling is also abrogated in IL-7α−/− mice. IL-7 is required for the development of 76 cells (Maki et al., 1996) and NK-T cells (Boesteanu et al., 1997).

In some embodiments, the IL-7 protein includes a polypeptide comprising the amino acid sequence as set forth in any one of SEQ ID NOs: 1 to 6. In other embodiments, the IL-7 protein comprises an amino acid sequence having a sequence identity of about 70%, about 75%, about 80%, about 85%, about 90%, about 91%, about 92%, about 93%, about 94%, about 95%, about 96%, about 97%, about 98%, or about 99% or higher, to an amino acid sequence set forth in SEQ ID NOs: 1 to 6.

In some embodiments, the IL-7 protein includes a modified IL-7 or a fragment thereof, wherein the modified IL-7 or the fragment retains one or more biological activities of wild-type IL-7. Non-limiting examples of such activities include (i) capable of binding to IL-7 receptor; (ii) inducing early T-cell development; (iii) promoting T-cell homeostasis. In some embodiments, the IL-7 protein can be derived from humans, rats, mice, monkeys, cows, or sheep.

In some embodiments, the human IL-7 can have an amino acid sequence represented by SEQ ID NO: 1 (GenBank Accession No. P13232); the rat IL-7 can have an amino acid sequence represented by SEQ ID NO: 2 (GenBank Accession No. P56478); the mouse IL-7 can have an amino acid sequence represented by SEQ ID NO: 3 (GenBank Accession No. P10168); the monkey IL-7 can have an amino acid sequence represented by SEQ ID NO: 4 (GenBank Accession No. NP 001279008); the cow IL-7 can have an amino acid sequence represented by SEQ ID NO: 5 (GenBank Accession No. P26895), and the sheep IL-7 can have an amino acid sequence represented by SEQ ID NO: 6 (GenBank Accession No. Q28540).

In other embodiments, the IL-7 protein useful for the present methods include an IL-7 fusion protein. In some embodiments, the IL-7 fusion protein can include an IL-7 protein and a heterologous moiety.

In some embodiments, the heterologous moiety can comprise a domain that includes an amino acid sequence having 1 to 10 amino acid residues (i.e., oligopeptide) consisting of methionine, glycine, or a combination thereof, e.g., MGM, fused to the N terminus or C terminus of IL-7. In certain embodiments, the oligopeptide is selected from the group consisting of methionine, glycine, methionine-methionine, glycine-glycine, methionine-glycine, glycine-methionine, methionine-methionine-methionine, methionine-methionine-glycine, methionine-glycine-methionine, glycine-methionine-methionine, methionine-glycine-glycine, glycine-methionine-glycine, glycine-glycine-methionine, and glycine-glycine-glycine. In some embodiments, the oligopeptide is methionine-glycine-methionine.

In other embodiments, the heterologous moiety comprises a moiety that can extend a half-life of IL-7 (“half-life extending moiety”). In some aspects, the present disclosure is directed to a method of treating a cancer in a subject in need thereof comprising administering to the subject a population of chimeric antigen receptor (CAR)-bearing immune effector cells, e.g., allogenic CAR-bearing immune effector cells or CAR-iNKT cells, and an IL-7 protein fused to a half-life extending moiety. In some aspects, the present disclosure is directed to a method of treating a cancer in a subject in need thereof comprising administering to the subject a population of chimeric antigen receptor (CAR)-bearing immune effector cells, e.g., allogenic CAR-bearing immune effector cells or CAR-iNKT cells, and an IL-7 protein fused to a half-life extending moiety, wherein the administration results in improved properties, e.g., increased anti-tumor efficacy, improved PK profile, and/or less toxicity, compared to a combination therapy of the CAR bearing immune effector cells and an IL-7 protein not fused to any half-life extending moiety.

In some embodiments, the IL-7 fusion protein comprises (i) IL-7 (a first domain), (ii) a second domain that includes an amino acid sequence having 1 to 10 amino acid residues (i.e., oligopeptide) consisting of methionine, glycine, or a combination thereof, e.g., MGM, and (iii) a third domain comprising a half-life extending moiety.

In some embodiments, the half-life extending moiety can be linked to the N-terminal or the C-terminal of the first domain or the second domain. Additionally, the IL-7 including the first domain and the second domain can be linked to both terminals of the third domain.

In some embodiments, the half-life extending moiety is a fusion partner for increasing in vivo half-life, and preferably, can be selected from the group consisting of an Fc region of immunoglobulin or a part thereof, albumin, an albumin binding polypeptide, Pro/Ala/Ser (PAS), C-terminal peptide(CTP) of˜subunit of human chorionic gonadotropin, polyethylene glycol (PEG), long unstructured hydrophilic sequences of amino acids (XTEN), hydroxyethyl starch(HES), an albumin-binding small molecule, and a combination thereof.

In some embodiments, the half-life extending moiety is Fc. In some aspects, the present disclosure is directed to a method of treating a cancer in a subject in need thereof comprising administering to the subject a population of chimeric antigen receptor (CAR)-bearing immune effector cells, e.g., allogenic CAR-bearing immune effector cells or CAR-iNKT cells, and an IL-7 protein fused to an Fc region. In some aspects, the present disclosure is directed to a method of treating a cancer in a subject in need thereof comprising administering to the subject a population of chimeric antigen receptor (CAR)-bearing immune effector cells, e.g., allogenic CAR-bearing immune effector cells or CAR-iNKT cells, and an IL-7 protein fused to an Fc region, wherein the administration results in improved properties, e.g., increased anti-tumor efficacy, improved PK profile, and/or less toxicity, compared to a combination therapy of the CAR bearing immune effector cells and an IL-7 protein not fused to any half-life extending moiety (e.g., not fused to an Fc region).

When the third domain is an Fc region of an immunoglobulin, in some embodiments, it can be an Fc region of a modified immunoglobulin. In particular, the Fc region of the modified immunoglobulin can be one in which the antibody-dependent cellular cytotoxicity (ADCC) or complement-dependent cytotoxicity (CDC) weakened due to the modification in the binding affinity with the cFc receptor and/or a complement. In some embodiments, the modified immunoglobulin can be selected from the group consisting of IgG1, IgG2, IgG3, IgG4, IgA1, IgA2, IgD, IgE and a combination thereof. Specifically, the Fc region of the modified immunoglobulin can include a hinge region, a CH2 domain, and a CH3 domain from theN-terminal to the C-terminal. In other embodiments, the hinge region can include the human IgD hinge region; the CH2 domain can include a part of the amino acid residues of the human IgD and a part of the amino acid residues of the human IgG4 CH2 domain; and the CH3 domain can include a part of the amino acid residues of the human IgG4 CH3 domain.

Additionally, in some embodiments, a fusion protein can form a dimer, for example, when the third domain is an Fc region, the Fc regions can bind to each other and thereby form a dimer.

As used herein, the terms “Fc region”, “Fc fragment”, or “Fc” refers to a protein which includes the heavy chain constant region 2 (CH2) and the heavy chain constant region 3 (CH3) of immunoglobulin but does not include its variable regions of the heavy chain and the light chain and the light chain constant region (CL), and it can further include a hinge region of the heavy chain constant region. A hybrid Fc or a hybrid Fc fragment thereof can be called “hFc” or “hyFc.” concept. Accordingly, in some embodiments, an Fc useful for the present disclosure is a hybrid Fc, comprising a hinge region, a CH2 domain, and a CH3 domain, wherein the hinge region comprise a human IgD hinge region, wherein the CD2 domain comprises a part of human IgD CH2 domain and a part of human IgG4 CH2 domain, and wherein the CH3 domain comprises a part of human IgG4 CH3 domain.

Additionally, as used herein, the term “an Fc region variant” refers to one which was prepared by substituting apart of the amino acids among the Fc region or by combining the Fc regions of different kinds. The Fc region variant can prevent from being cut off at the hinge region. Specifically, the 144th amino acid and/or 145th amino acid of SEQ ID NO:9 can be modified. See US20170158746, which is incorporated herein by reference in its entirety. Preferably, the variant can be one, in which the 144th amino acid, K, was substituted with G or S, and one, in which the 145th amino acid, E, was substituted with G or S.

Additionally, in some embodiments, the Fc fragment can be in the form of having native sugar chains, increased sugar chains, or decreased sugar chains compared to the native form, or can be in a deglycosylated form. In some embodiments, the immunoglobulin Fc sugar chains can be modified by conventional methods such as a chemical method, an enzymatic method, and a genetic engineering method using a microorganism. The removal of sugar chains from an Fc fragment results in a sharp decrease in binding affinity to the C1q part of the first complement component C1, and a decrease or loss of ADCC or CDC, thereby not inducing any unnecessary immune responses in vivo. In this regard, an immunoglobulin Fc region in a deglycosylated or a glycosylated form can be more suitable as a drug carrier.

The Fc region of the modified immunoglobulin can be one described in U.S. Pat. No. 7,867,491, and the production of the Fc region of the modified immunoglobulin can be performed referring to the disclosure in U.S. Pat. No. 7,867,491.

In some embodiments, an IL-7 protein can be fused to albumin, a variant, or a fragment thereof. Examples of the IL-7-albumin fusion protein can be found at International Application Publication No. WO 2011/124718 A1. In some embodiments, an IL-7 protein is fused to a pre-pro-B cell Growth Stimulating Factor (PPBSF), optionally by a flexible linker. See US 2002/0058791A1. In other embodiments, an IL-7 protein useful for the disclosure is an IL-7 conformer that has a particular three dimensional structure. See US 2005/0249701 A1. In some embodiments, an IL-7 protein can be fused to an Ig chain, wherein amino acid residues 70 and 91 in the IL-7 protein are glycosylated the amino acid residue 116 in the IL-7 protein is non-glycosylated. See U.S. Pat. No. 7,323,549 B2. In some embodiments, an IL-7 protein that does not contain potential T-cell epitopes (thereby to reduce anti-IL-7 T-cell responses) can also be used for the present disclosure. See US 2006/0141581 A1. In other embodiments, an IL-7 protein that has one or more amino acid residue mutations in carboxy-terminal helix D region can be used for the present disclosure. The IL-7 mutant can act as IL-7R partial agonist despite lower binding affinity for the receptor. See US 2005/0054054A1. Any IL-7 proteins described in the above listed patents or publications are incorporated herein by reference in their entireties.

In addition, non-limiting examples of additional IL-7 proteins useful for the present disclosure are described in U.S. Pat. Nos. 7,708,985, 8,034,327, 8,153,114, 7,589,179, 7,323,549, 7,960,514, 8,338,575, 7,118,754, 7,488,482, 7,670,607, 6,730,512, WO0017362, GB2434578A, WO 2010/020766 A2, WO91/01143, Beq et al., Blood, vol. 114 (4), 816, 23 Jul. 2009, Kang et al., J. Virol. Doi:10.1128/JVI.02768-15, Martin et al., Blood, vol. 121 (22), 4484, May 30, 2013, McBride et al., Acta Oncologica, 34:3, 447-451, Jul. 8, 2009, and Xu et al., Cancer Science, 109: 279-288, 2018, which are incorporated herein by reference in their entireties.

In some embodiments, the second domain can be directly linked to the N-terminal of the first domain or linked by a linker. Specifically, the result can be in the form of the second domain-the first domain or the second domain-linker-the first domain.

In some embodiments, the third domain can be directly linked to the first domain or the second domain or linked by a linker. Specifically, the result can be in the form of the second domain-the first domain-the third domain, the third domain-the second domain-the first domain, the second domain-the first domain-linker-the third domain, the third domain-linker-the second domain-the first domain, the second domain-linker-the first domain-linker-the third domain, or the third domain-linker-the second domain-the first domain.

When the linker is a peptide linker, in some embodiments, the connection can occur in any linking region. They can be coupled using a crosslinking agent known in the art. In some embodiments, examples of the crosslinking agent can include N-hydroxy succinimide esters such as 1,1-bis(diazoacetyl)-2-phenylethane, glutaraldehyde, and 4-azidosalicylic acid; imido esters including disuccinimidyl esters such as 3,3′-dithiobis (succinimidyl propionate), and bifunctional maleimides such as bis-Nmaleimido-1,8-octane, but is not limited thereto.

Additionally, in some embodiments, the linker can be an albumin linker or a peptide linker. The peptide linker can be a peptide of 10 to 20 amino acid residues consisting of Gly and Ser residues.

When the linker is formed by one selected from the group consisting of a chemical bond, in some embodiments, the chemical bond can be a disulfide bond, a diamine bond, a sulfide-amine bond, a carboxy-amine bond, an ester bond, and a covalent bond.

In some embodiments, the IL-7 protein can be modified and have a structure of (A)-(IL-7), wherein (IL-7) is a polypeptide having a biological activity of IL-7 and (A) is an oligopeptide consisting of 1 to 10 amino acids. As used herein, the term “a polypeptide having a biological activity of IL-7” refers to a polypeptide or protein having the same or similar sequence and activity to IL-7. Unless otherwise specified in the present invention, the term can be used as a concept which is interchangeable with the first domain of the IL-7 fusion proteins.

In some embodiments, the IL-7 protein having the structure described above can have an amino acid sequence chosen from SEQ ID NOs: 15 to 20. In certain embodiments, the IL-7 protein can comprise an amino acid sequence having a homology of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, and at least about 99%, to the amino acid sequences of SEQ ID NOS: 15 to 20.

In some embodiments, the IL-7 fusion protein comprises: a first domain including a polypeptide having the activity of IL-7 or a similar activity thereof, a second domain comprising an amino acid sequence having 1 to 10 amino acid residues consisting of methionine, glycine, or a combination thereof (i.e., oligopeptide); and a third domain, which is an Fc region of modified immunoglobulin, coupled to the C-terminal of the first domain.

The IL-7 fusion protein can have an amino acid sequence chosen from SEQ ID NOs: 21 to 25. Additionally, in some embodiments, the IL-7 fusion protein comprises an amino acid sequence having a homology of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, and at least about 99% to amino acid 2 to 153 of SEQ ID NO: 21, amino acids 3 to 154 of SEQ ID NO: 22 or 23, amino acids 4 to 155 of SEQ ID NO: 24, or amino acids 5 to 156 of SEQ ID NO: 25. In some embodiments, the IL-7 fusion protein comprises an amino acid sequence having a homology of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, and at least about 99% to amino acids 4 to 155 of SEQ ID NO: 24.

In some embodiments, the IL-7 fusion protein comprises an amino acid sequence having a homology of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, and at least about 99% to amino acids 1 to 153 of SEQ ID NO: 21, amino acids 1 to 154 of SEQ ID NO: 22 or 23, amino acids 1 to 155 of SEQ ID NO: 24, or amino acids 1 to 156 of SEQ ID NO: 25. In some embodiments, the IL-7 fusion protein comprises an amino acid sequence having a homology of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, and at least about 99% to amino acids 1 to 155 of SEQ ID NO: 24.

In some embodiments, the IL-7 fusion protein comprises an amino acid sequence having a homology of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, and at least about 99% to amino acids 4 to 400 of SEQ ID NO: 24.

In some embodiments, the IL-7 fusion protein comprises an amino acid sequence having a homology of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, and at least about 99% to an amino acid sequence of SEQ ID NOS: 21 to 25. In some embodiments, the IL-7 fusion protein comprises an amino acid sequence having a homology of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, and at least about 99% to the amino acid as set forth as SEQ ID NO: 24.

In some embodiments, an IL-7 protein can be fused to albumin, a variant, or a fragment thereof. Examples of the IL-7-albumin fusion protein can be found at International Application Publication No. WO 2011/124718 A1. In some embodiments, an IL-7 protein is fused to a pre-pro-B cell Growth Stimulating Factor (PPBSF), optionally by a flexible linker. See US 2002/0058791A1. In other embodiments, an IL-7 protein useful for the disclosure is an IL-7 conformer that has a particular three dimensional structure. See US 2005/0249701 A1. In some embodiments, an IL-7 protein can be fused to an Ig chain, wherein amino acid residues 70 and 91 in the IL-7 protein are glycosylated the amino acid residue 116 in the IL-7 protein is non-glycosylated. See U.S. Pat. No. 7,323,549 B2. In some embodiments, an IL-7 protein that does not contain potential T-cell epitopes (thereby to reduce anti-IL-7 T-cell responses) can also be used for the present disclosure. See US 2006/0141581 A1. In other embodiments, an IL-7 protein that has one or more amino acid residue mutations in carboxy-terminal helix D region can be used for the present disclosure. The IL-7 mutant can act as IL-7R partial agonist despite lower binding affinity for the receptor. See US 2005/0054054A1. Any IL-7 proteins described in the above listed patents or publications are incorporated herein by reference in their entireties.

In addition, non-limiting examples of additional IL-7 proteins useful for the present disclosure are described in U.S. Pat. Nos. 7,708,985, 8,034,327, 8,153,114, 7,589,179, 7,323,549, 7,960,514, 8,338,575, 7,118,754, 7,488,482, 7,670,607, 6,730,512, WO0017362, GB2434578A, WO 2010/020766 A2, WO91/01143, Beq et al., Blood, vol. 114 (4), 816, 23 Jul. 2009, Kang et al., J. Virol. Doi:10.1128/JVI.02768-15, Martin et al., Blood, vol. 121 (22), 4484, May 30, 2013, McBride et al., Acta Oncologica, 34:3, 447-451, Jul. 8, 2009, and Xu et al., Cancer Science, 109: 279-288, 2018, which are incorporated herein by reference in their entireties.

In some embodiments, the IL-7 protein is encoded by an isolated nucleic acid molecule encoding the IL-7 protein. The nucleic acid molecule can be one encoding the polypeptide having an amino acid sequence chosen from SEQ ID NOS: 15 to 25, or one with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, and at least about 99% homology to those sequences. The nucleic acid molecule can include a polynucleotide sequence chosen from SEQ ID NOS: 29 to 39, or one with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, and at least about 99% homology to those sequences. The nucleic acid molecule can further include a signal sequence or a leader sequence. The characteristics of the signal peptides are well known in the art, and the signal peptides conventionally having 16 to 30 amino acids, but they can include more or less number of amino acid residues. Conventional signal peptides consist of three regions of the basic N-terminal region, a central hydrophobic region, and a more polar C-terminal region.

In some embodiments, the central hydrophobic region includes 4 to 12 hydrophobic residues, which immobilize the signal sequence through a membrane lipid bilayer during the translocation of an immature polypeptide. After the initiation, the signal sequence can be frequently cut off within the lumen of ER by a cellular enzyme known as a signal peptidase. In particular, the signal sequence can be a secretory signal sequence for tissue plasminogen activation (tPa), signal sequence of herpes simplex virus glycoprotein D (HSV gDs), or a growth hormone. Preferably, the secretory signal sequence used in higher eukaryotic cells including mammals, etc., can be used. Additionally, in some embodiments, as the secretory signal sequence, the signal sequence included in the wild type IL-7 can be used or it can be used after substituting with a codon with high expression frequency in a host cell.

The IL-7 protein useful for the present disclosure, in some embodiments, can be encoded by an expression vector comprising an isolated nucleic acid molecule encoding the IL-7 protein. The expression vector can be RcCMV (Invitrogen, Carlsbad) or a variant thereof. The expression vector can include a human cytomegalovirus (CMV) for promoting continuous transcription of a target gene in a mammalian cell and a polyadenylation signal sequence of bovine growth hormone for increasing the stability state of RNA after transcription. In some embodiments, the expression vector is pAD15, which is a modified form of RcCMV.

The IL-7 protein useful for the present disclosure, in some embodiments, can be expressed by a host cell including the expression vector. An appropriate host cell can be used for the expression and/or secretion of a target protein, by the transduction or transfection of the DNA sequence.

Examples of the appropriate host cell to be used, in some embodiments, can include immortal hybridoma cell, NS/0 myeloma cell, 293 cell, Chinese hamster ovary (CHO)cell, HeLa cell, human amniotic fluid-derived cell (Cap T cell) or COS cell.

The IL-7 protein useful for the disclosure, in some embodiments, can be made by culturing the transformed cells by the expression vector; and harvesting the IL-7 protein from the culture or the cells obtained from the culturing process.

The IL-7 protein useful for the disclosure, in some embodiments, can be purified from the culture medium or cell extract. For example, after obtaining the supernatant of the culture medium, in which a recombinant protein was secreted, the supernatant can be concentrated a protein concentration filter available in the commercial market, e.g., an Amicon or Millipore Pellicon ultrafiltration unit. Then, the concentrate can be purified by a method known in the art. For example, the purification can be performed using a matrix coupled to protein A.

The IL-7 protein useful for the disclosure, in some embodiments, can be prepared by including a linking oligopeptide of an amino acid sequence having 1 to 10 amino acid residues consisting of methionine, glycine, or a combination thereof, to the N-terminal of a polypeptide having the activity of IL-7 or a similar activity thereof.

The above preparation method can further include a step of linking a polynucleotide encoding a polypeptide consisting of a heterogeneous sequence with an IL-7 protein. In particular, the polypeptide consisting of a heterogeneous sequence can be any one selected from the group consisting of an Fc region of immunoglobulin or a part thereof, albumin, an albumin binding polypeptide, PAS, a CTP of the 3 subunit of human chorionic gonadotropin, PEG, XTEN, HES, an albumin binding small molecule, and a combination thereof.

The IL-7 protein can be administered for promoting the expansion or survival of chimeric antigen receptor (CAR)-bearing immune effector cells, in particular, engineered chimeric antigen receptor bearing T cells (CAR-Ts) and/or CAR-bearing iNKT cells (CAR-iNKTs).

Therefore, in some aspects, the present disclosure is directed to a method of treating a cancer in a subject in need thereof comprising administering to the subject a population of allogenic CAR-bearing immune effector cells, and an IL-7 protein fused to a half-life extending moiety, e.g., an Fc region. In some aspects, the present disclosure is directed to a method of treating a cancer in a subject in need thereof comprising administering to the subject a population of allogenic CAR-bearing immune effector cells, and an IL-7 protein fused to a half-life extending moiety, e.g., an Fc region, wherein the administration results in improved properties, e.g., increased anti-tumor efficacy and/or less toxicity, compared to the allogenic CAR-bearing immune effector cells alone or an IL-7 protein alone.

In some aspects, the present disclosure is directed to a method of treating a cancer in a subject in need thereof comprising administering to the subject a population of CAR-iNKT cells, and an IL-7 protein fused to an Fc region. In some aspects, the present disclosure is directed to a method of treating a cancer in a subject in need thereof comprising administering to the subject a population of CAR-iNKT cells, and an IL-7 protein fused to an Fc region, wherein the administration results in improved properties, e.g., increased anti-tumor efficacy and/or less toxicity, compared to the CAR-iNKT cells or the IL-7 protein alone.

The IL-7 protein useful for the disclosure, in some embodiments, further include a pharmaceutically acceptable carrier. The pharmaceutically acceptable carrier can be any non-toxic material which is suitable for the delivery into patients. The carrier can be distilled water, alcohols, fats, waxes, or inactive solids. Additionally, any pharmaceutically acceptable adjuvants (buffering agents, dispersing agents) can also be contained therein.

Additionally, the pharmaceutical composition containing the IL-7 protein can be administered to subjects by various methods. For example, the composition can be parenterally administered, e.g., subcutaneously, intramuscularly, or intravenously, e.g., intramuscularly. The composition can be sterilized by a conventional sterile method. The composition can contain a pharmaceutically acceptable auxiliary material and an adjuvant required for the regulation of physiological conditions such as pH adjustment, a toxicity-adjusting agent, and an analog thereof. Specific examples can include sodium acetate, potassium chloride, calcium chloride, sodium lactate, etc. The concentration of the fusion protein to be included in the formulations can vary widely. For example, the concentration of the fusion protein can be less than about 0.5%, and generally or at least about 1% to as much as 15% to 20%, depending on the weight. The concentration can be selected based on the selected particular administration methods, fluid volumes, viscosities, etc.

The present method includes administering a therapeutically effective amount of the IL-7 protein in combination with a population of CAR-bearing immune effector cells, e.g., CAR T cells, to a subject in need thereof, who has a health state related or unrelated to the target disease. The subject can be a mammal, and preferably a human.

Compositions can be administered by appropriate routes. Compositions can be provided by a direct administration (e.g., locally by an administration via injection, transplantation, or local administration into a tissue region) or system (e.g., parenterally or orally) via an appropriate means. In some embodiments, the IL-7 protein can be administered intravenously, subcutaneously, intraocularly, intraperitoneally, intramuscularly, orally, intrarectally, intraorbitally, intracerebrally, intracranially, intraspinally, intraventricularly, intrathecally, intracistenally, intracapsularly, intranasally, or aerosol administration. In other embodiments, the composition is formulated to contain an aqueous or physiologically applicable suspension of body fluids or a part of the solution thereof. As such, the physiologically acceptable carrier or transporter can be added into the composition and delivered to patients, and this does not cause a negative effect on the electrolyte and/or volume balance of patients. Accordingly, the physiologically acceptable carrier or transporter can be a physiological saline. CAR-immune effector cells will, of course, be administered by injection or infusion, typically intravenously.

For reconstituting or complementing the functions of a desired protein, an expression vector capable of expressing a fusion protein in a particular cell can be administered along with any biologically effective carrier. This can be any formulation or composition that can efficiently deliver a gene encoding a desired protein or an IL-7 fusion protein into a cell in vivo.

The unit dose of the modified IL-7 or an IL-7 fusion protein can be in the range of 0.001 mg/kg to 10 mg/kg. In one embodiment, a therapeutically effective amount of the IL-7 protein to be used in combination therapy with a population of CAR-bearing immune effector cells, e.g., CAR T cells, can be in the range of 0.01 mg/kg to 2 mg/kg. In another embodiment, the therapeutically effective amount of the protein, for humans, can be in the range of 0.02 mg/kg to 1 mg/kg, e.g., 20 g/kg to 600 g/kg, e.g., 60 g/kg to 600 g/kg, e.g., 2,000 μg/kg. In some embodiments, a therapeutically effective amount of an IL-7 protein is about 10 mg/kg. In other embodiments, a therapeutically effective amount of an IL-7 protein is about 20 μg/kg, about 60 μg/kg, about 120 μg/kg, about 240 μg/kg, about 480 μg/kg, or about 600 μg/kg or more (e.g., 2,000 μg/kg). In other embodiments, a therapeutically effective amount of an IL-7 protein is about a flat dose of about 0.25 mg, about 1 mg, about 3 mg, about 6 mg, or about 9 mg. In other embodiments, a therapeutically effective amount of an IL-7 protein is a flat dose. In some embodiments, a therapeutically effective amount of an IL-7 protein is about 0.25 mg to about 9 mg, e.g., about 0.25 mg, about 1 mg, about 3 mg, about 6 mg, or about 9 mg. In some embodiments, the therapeutically effective amount can vary depending on the subject diseases for treatment and the presence of adverse effects. In some embodiments, the administration of the IL-7 protein can be performed by periodic bolus injections or external reservoirs (e.g., intravenous bags) or by continuous intravenous, subcutaneous, or intraperitoneal administration from the internal (e.g., biocorrosive implants).

In certain embodiments, the IL-7 protein is administered at a dosing interval of at least a week, at least two weeks, at least three weeks, at least four weeks, at least five weeks, at least six weeks, at least seven weeks, at least eight weeks, at least nine weeks, or at least ten weeks.

In other embodiments, the IL-7 protein can be administered repeatedly. In other embodiments, the IL-7 protein is administered at least two times, at least three times, at least four times, at least five times, at least six times, at least seven times, at least eight times, at least nine times, or at least ten times.

In certain embodiments, the IL-7 protein can be formulated: for example, about 3 mg/ml to about 100 mg/ml an IL-7 protein, about 20 mM sodium citrate, about 5 w/v % sucrose, about 1 to 2 w/v % sorbitol or mannitol, and about 0.05 w/v % Tween 80 or poloxamer at a pH of about 5.0.

In some embodiments, the IL-7 protein and CAR-bearing immune effector cells can be administered in combination with other drug(s) or physiologically active material(s) which have a preventative or treating effect on the disease to be prevented or treated, or can be formulated into a combined preparation in combination with other drug(s), for example, can be administered in combination with an immunostimulant such as a hematopoietic growth factor, a cytokine, an antigens, and an adjuvant. The hematopoietic growth factor can be a stem cell factor (SCF), a G-CSF, a GM-CSF, or an Flt-3 ligand. The cytokine can be γ interferon, IL-2, IL-15, IL-21, IL-12, RANTES, or B7-1.

### III. Chimeric Antigen Receptor (CAR) Bearing Immune Effector Cells

### A. Mono CAR-T Cells

A CAR-T cell is a T cell that expresses a chimeric antigen receptor. The phrase “chimeric antigen receptor (CAR),” as used herein, refers to a recombinant fusion protein that has an antigen-specific extracellular domain coupled to an intracellular domain that directs the cell to perform a specialized function upon binding of an antigen to the extracellular domain. The terms “artificial T cell receptor,” “chimeric T-cell receptor,” and “chimeric immunoreceptor” can each be used interchangeably herein with the term “chimeric antigen receptor.” Chimeric antigen receptors are distinguished from other antigen binding agents by their ability to both bind MHC-independent antigen and transduce activation signals via their intracellular domain. The extracellular and intracellular portions of a CAR are discussed in more detail below.

The antigen-specific extracellular domain of a chimeric antigen receptor recognizes and specifically binds an antigen, typically a surface-expressed antigen of a malignancy. An antigen-specific extracellular domain specifically binds an antigen when, for example, it binds the antigen with an affinity constant or affinity of interaction (KD) between about 0.1 pM to about 10 IJM, preferably about 0.1 pM to about 1 1-JM, more preferably about 0.1 pM to about 100 nM. Methods for determining the affinity of interaction are known in the art. An antigen-specific extracellular domain suitable for use in a CAR of the present disclosure can be any antigen-binding polypeptide, a wide variety of which are known in the art. In some instances, the antigen-binding domain is a single chain Fv (scFv). Other antibody-based recognition domains (cAb VHH (camelid antibody variable domains) and humanized versions thereof, lgNAR VH (shark antibody variable domains) and humanized versions thereof, sdAb VH (single domain antibody variable domains) and “camelized” antibody variable domains are suitable for use. In some instances,T-cell receptor (TCR) based recognition domains such as single chain TCR (scTv, single chain two-domain TCR containing V.alpha.V.beta.) are also suitable for use.

Suitable antigens can include T cell-specific antigens and/or antigens that are not specific to T cells. In one preferred embodiment, an antigen specifically bound by the chimeric antigen receptor of a CAR-T cell, and the antigen for which the CAR-T cell is deficient, is an antigen expressed on a malignant T cell, more preferably an antigen that is overexpressed on malignant T cell in comparison to a non-malignant T cell. A “malignant T cell” is a T cell derived from a T-cell malignancy. The term “T-cell malignancy” refers to a broad, highly heterogeneous grouping of malignancies derived from T-cell precursors, mature T cells, or natural killer cells. Non-limiting examples of T-cell malignancies include T-cell acute lymphoblastic leukemia/lymphoma (T-ALL), T-cell large granular lymphocyte (LGL) leukemia, human T-cell leukemia virus type 1-positive (HTLV-1+) adult T-cell leukemia/lymphoma (ATL), T-cell prolymphocytic leukemia (T-PLL), and various peripheral T-cell lymphomas (PTCLs), including but not limited to angioimmunoblastic T-cell lymphoma (AITL), ALK positive anaplastic large cell lymphoma, and ALK-negative anaplastic large cell lymphoma.

Suitable CAR antigens can also include antigens found on the surface of a multiple myeloma cell, i.e., a malignant plasma cell, such as B-Cell Maturation Antigen (BCMA), CS1, CD38, and CD19.

Alternatively, the CAR can be designed to express the extracellular portion of the APRIL protein, the ligand for BCMA and TACI, effectively co-targeting both BCMA and TACI for the treatment of multiple myeloma.

In some embodiments, suitable CAR antigens can include antigens expressed on cells associated with a leukemia, e.g., acute myeloid leukemia. A non-limiting example of such antigens includes C-type lectin-like molecule-1 (CLL-1).

For instance, by way of non-limiting example, CD2, CD3R, CD4, CD5, CD7, TRAC, TCRβ, BCMA, CLL-1, CS1, CD38, and CD19 can be antigens expressed on a malignant T cell. In one embodiment, a CAR-T cell of the present disclosure comprises an extracellular domain of a chimeric antigen receptor that specifically binds to CD2. In another embodiment, a CAR-T cell of the present disclosure comprises an extracellular domain of a chimeric antigen receptor that specifically binds to CD38. In another embodiment, a CAR-T cell of the present disclosure comprises an extracellular domain of a chimeric antigen receptor that specifically binds to CD4. In another embodiment, a CAR-T cell of the present disclosure comprises an extracellular domain of a chimeric antigen receptor that specifically binds to CD5. In yet another embodiment, a CAR-T cell of the present disclosure comprises an extracellular domain of a chimeric antigen receptor that specifically binds to CD7. In yet another embodiment, a CAR-T cell of the present disclosure comprises an extracellular domain of a chimeric antigen receptor that specifically binds to TRAC. In yet another embodiment, a CAR-T cell of the present disclosure comprises an extracellular domain of a chimeric antigen receptor that specifically binds to TCRβ. In still another embodiment, a CAR-T cell of the present disclosure comprises an extracellular domain of a chimeric antigen receptor that specifically binds to BCMA. In other embodiments, a CAR-T cell of the present disclosure comprises an extracellular domain of a chimeric antigen receptor that specifically binds to CLL-1. In still another embodiment, a CAR-T cell of the present disclosure comprises an extracellular domain of a chimeric antigen receptor that specifically binds to CS1. In still another embodiment, a CAR-T cell of the present disclosure comprises an extracellular domain of a chimeric antigen receptor that specifically binds to CD38. In still yet another embodiment, a CAR-T cell of the present disclosure comprises an extracellular domain of a chimeric antigen receptor that specifically binds to CD19.

A chimeric antigen receptor of the present disclosure also comprises an intracellular domain that provides an intracellular signal to the T cell upon antigen binding to the antigen-specific extracellular domain. The intracellular signaling domain of a chimeric antigen receptor of the present disclosure is responsible for activation of at least one of the effector functions of the T cell in which the chimeric receptor is expressed.

The term “intracellular domain” refers to the portion of a CAR that transduces the effector function signal upon binding of an antigen to the extracellular domain and directs the T cell to perform a specialized function. Non-limiting examples of suitable intracellular domains include the zeta chain of the T-cell receptor or any of its homologs (e.g., eta, delta, gamma, or epsilon), MB 1 chain, 829, Fc RIII, Fc RI, and combinations of signaling molecules, such as CD3.zeta. and CD28, CD27, 4-1 BB, DAP-1 0, OX40, and combinations thereof, as well as other similar molecules and fragments. Intracellular signaling portions of other members of the families of activating proteins can be used, such as FcγRIII and FcεRI. While usually the entire intracellular domain will be employed, in many cases it will not be necessary to use the entire intracellular polypeptide. To the extent that a truncated portion of the intracellular signaling domain can find use, such truncated portion can be used in place of the intact chain as long as it still transduces the effector function signal. The term intracellular domain is thus meant to include any truncated portion of the intracellular domain sufficient to transduce the effector function signal. Typically, the antigen-specific extracellular domain is linked to the intracellular domain of the chimeric antigen receptor by a transmembrane domain. A transmembrane domain traverses the cell membrane, anchors the CAR to the T cell surface, and connects the extracellular domain to the intracellular signaling domain, thus impacting expression of the CAR on the T cell surface. Chimeric antigen receptors can also further comprise one or more costimulatory domain and/or one or more spacer. A costimulatory domain is derived from the intracellular signaling domains of costimulatory proteins that enhance cytokine production, proliferation, cytotoxicity, and/or persistence in vivo. A “peptide hinge” connects the antigen-specific extracellular domain to the transmembrane domain. The transmembrane domain is fused to the costimulatory domain, optionally a costimulatory domain is fused to a second costimulatory domain, and the costimulatory domain is fused to a signaling domain, not limited to CD3ζ. For example, inclusion of a spacer domain between the antigen-specific extracellular domain and the transmembrane domain, and between multiple scFvs in the case of tandem CAR, can affect flexibility of the antigen-binding domain(s) and thereby CAR function. Suitable transmembrane domains, costimulatory domains, and spacers are known in the art. In a similar manner, other mono CAR-T cells can be constructed, and are given below in Table 3 and Table 5.

### B. Genome-Edited CAR-T Cells

The CAR-T cells encompassed by the present disclosure are deficient in one or more antigens to which the chimeric antigen receptor specifically binds and are therefore fratricide-resistant. In some embodiments, the one or more antigens of the T cell is modified such the chimeric antigen receptor no longer specifically binds the one or more modified antigens. For example, the epitope of the one or more antigens recognized by the chimeric antigen receptor can be modified by one or more amino acid changes (e.g., substitutions or deletions) or the epitope can be deleted from the antigen. In other embodiments, expression of the one or more antigens is reduced in the T cell by at least 50%, at least 60%, at least 70%, at least 80%, at least 90% or more. Methods for decreasing the expression of a protein are known in the art and include, but are not limited to, modifying or replacing the promoter operably linked to the nucleic acid sequence encoding the protein. In still other embodiments, the T cell is modified such that the one or more antigens is not expressed, e.g., by deletion or disruption of the gene encoding the one or more antigens. In each of the above embodiments, the CAR-T cell can be deficient in one or preferably all the antigens to which the chimeric antigen receptor specifically binds. Methods for genetically modifying a T cell to be deficient in one or more antigens are well known in art. In an exemplary embodiment, CRISPR/cas9 gene editing can be used to modify a T cell to be deficient in one or more antigens.

CAR-T cells encompassed by the present disclosure can further be deficient in endogenous T cell receptor (TCR) signaling as a result of deleting a part of the T Cell Receptor (TCR)-CD3 complex. In various embodiments it can be desirable to eliminate or suppress endogenous TCR signaling in CAR-T cells disclosed herein. For example, decreasing or eliminating endogenous TCR signaling in CAR-T cells can prevent or reduce graft versus host disease (GvHD) when allogenic T cells are used to produce the CAR-T cells. Methods for eliminating or suppressing endogenous TCR signaling are known in the art and include, but are not limited to, deleting a part of the TCR-CD3 receptor complex, e.g., the TCR receptor alpha chain (TRAC), the TCR receptor beta chain (TRBC), CD3.epsilon, CD3.gamma, CD3.delta, and/or CD3.gamma. Deleting a part of the TCR receptor complex can block TCR mediated signaling and can thus permit the safe use of allogeneic T cells as the source of CAR-T cells without inducing life-threatening GvHD.

Alternatively, or in addition, CAR-T cells encompassed by the present disclosure can further comprise one or more suicide genes. As used herein, “suicide gene” refers to a nucleic acid sequence introduced to a CAR-T cell by standard methods known in the art that, when activated, results in the death of the CAR-T cell. Suicide genes can facilitate effective tracking and elimination of the CAR-T cells in vivo if required. Facilitated killing by activating the suicide gene can occur by methods known in the art. Suitable suicide gene therapy systems known in the art include, but are not limited to, various the herpes simplex virus thymidine kinase (HSVtk)/ganciclovir (GCV) suicide gene therapy systems or inducible caspase 9 protein. In an exemplary embodiment, a suicide gene is a CD34/thymidine kinase chimeric suicide gene.

In a similar manner, other mono CAR-T cells can be constructed, and are given below in Tables 3-6.

### C. Dual CAR-T Cells

A genome-edited, dual CAR-T cell, i.e., CD2*CD3e-dCARTΔCD2ΔCD38, can be generated by cloning a commercially synthesized anti-CD2 single chain variable fragment into a lentiviral vector containing a 3rd generation CAR backbone with CD28 and 4-1BB internal signaling domains and cloning a commercially synthesized anti-CD3e single chain variable into the same lentiviral vector containing an additional 3rd generation CAR backbone with CD28 and 4-1BB internal signaling domains resulting in a plasmid from which the two CAR constructs are expressed from the same vector.

In one embodiment, the disclosure provides an engineered T cell comprising a dual Chimeric Antigen Receptor (dCAR), i.e., two CARs expressed from a single lentivirus construct, that specifically binds CD5 and TCR receptor alpha chain (TRAC), wherein the T cell is deficient in CD5 and TRAC (e.g., CD5*TRAC-dCARTΔCD5ΔTRAC cell). In non-limiting examples the deficiency in CD5 and the TCR receptor alpha chain (TRAC) resulted from (a) modification of CD5 and the TCR receptor alpha chain (TRAC) expressed by the T cell such that the chimeric antigen receptor no longer specifically binds the modified CD5 and the TCR receptor alpha chain (TRAC), (b) modification of the T cell such that expression of the CD5 and the TCR receptor alpha chain (TRAC) is reduced in the T cell by at least 50%, at least 60%, at least 70%, at least 80%, at least 90% or more, or (c) modification of the T cell such that CD5 and the TCR receptor alpha chain (TRAC) is not expressed (e.g., by deletion or disruption of the gene encoding CD5 and/or the TCR receptor alpha chain (TRAC). In further embodiments, the T cell comprises a suicide gene. In non-limiting examples the suicide gene expressed in the CD5*TRAC-CARTΔCD5ΔTRAC cells encodes a modified Human-Herpes Simplex Virus-1-thymidine kinase (TK) gene fused in-frame to the extracellular and transmembrane domains of the human CD34 eDNA.

In a second embodiment, the disclosure provides an engineered T cell compromising a dCAR that specifically binds CD7 and TCR receptor alpha chain (TRAC), wherein the T cell is deficient in CD7 and TRAC (e.g., CD7*TRAC-dCARTΔCD7ΔTRAC cell). In non-limiting examples the deficiency in CD7 and the TCR receptor alpha chain (TRAC) resulted from (a) modification of CD5 and the TCR receptor alpha chain (TRAC) expressed by the T cell such that the chimeric antigen receptor no longer specifically binds the modified CD7 and the TCR receptor alpha chain (TRAC), (b) modification of the T cell such that expression of the CD7 and the TCR receptor alpha chain (TRAC) is reduced in the T cell by at least 50%, at least 60%, at least 70%, at least 80%, at least 90% or more, or (c) modification of the T cell such that CD7 and the TCR receptor alpha chain (TRAC) is not expressed (e.g., by deletion or disruption of the gene encoding CD7 and/or the TCR receptor alpha chain (TRAC). In further embodiments, the T cell comprises a suicide gene. In non-limiting examples the suicide gene expressed in the CD7*TRAC-dCARTΔCD7ΔTRAC cells encodes a modified Human-Herpes Simplex Virus-1-thymidine kinase (TK) gene fused in-frame to the extracellular and transmembrane domains of the human CD34 eDNA.

In a third embodiment, the disclosure provides an engineered T cell compromising a dCAR that specifically binds CD2 and TCR receptor alpha chain (TRAC), wherein the T cell is deficient in CD2 and TRAC (e.g., CD2*TRAC-dCARTΔCD2ΔTRAC cell). In non-limiting examples the deficiency in CD2 and the TCR receptor alpha chain (TRAC) resulted from (a) modification of CD2 and the TCR receptor alpha chain (TRAC) expressed by the T cell such that the chimeric antigen receptor no longer specifically binds the modified CD2 and the TCR receptor alpha chain (TRAC), (b) modification of the T cell such that expression of the CD7 and the TCR receptor alpha chain (TRAC) is reduced in the T cell by at least 50%, at least 60%, at least 70%, at least 80%, at least 90% or more, or (c) modification of the T cell such that CD2 and the TCR receptor alpha chain (TRAC) is not expressed (e.g., by deletion or disruption of the gene encoding CD2 and/or the TCR receptor alpha chain (TRAC). In further embodiments, the T cell comprises a suicide gene. In non-limiting examples the suicide gene expressed in the CD2*TRAC-dCARTΔCD2ΔTRAC cells encodes a modified Human-Herpes Simplex Virus-1-thymidine kinase (TK) gene fused in-frame to the extracellular and transmembrane domains of the human CD34 eDNA.

In a similar manner, other dual CAR-T cells can be constructed, and are given below in Table 4-5.

### D. Tandem CAR-T Cells

A tandem CAR-T cell (equivalently, tCAR-T), is a T cell with a single chimeric antigen polypeptide containing two distinct antigen recognition domains with affinity to different targets wherein the antigen recognition domain are linked through a peptide linker and share common costimulatory domain (s), wherein binding of either antigen recognition domain will signal though a common costimulatory domains(s) and signaling domain.

In one embodiment, the disclosure provides an engineered T cell comprising a tandem Chimeric Antigen Receptor (tCAR), i.e., two scFv sharing a single intracellular domain, that specifically binds CD5 and TCR receptor alpha chain (TRAC), wherein the T cell is deficient in CD5 and TRAC (e.g., CD5*TRAC-tCARTΔCD5ΔTRAC cell). In non-limiting examples the deficiency in CD5 and the TCR receptor alpha chain (TRAC) resulted from (a) modification of CD5 and the TCR receptor alpha chain (TRAC) expressed by the T cell such that the chimeric antigen receptor no longer specifically binds the modified CD5 and the TCR receptor alpha chain (TRAC), (b) modification of the T cell such that expression of the CD5 and the TCR receptor alpha chain (TRAC) is reduced in the T cell by at least 50%, at least 60%, at least 70%, at least 80%, at least 90% or more, or (c) modification of the T cell such that CD5 and the TCR receptor alpha chain (TRAC) is not expressed (e.g., by deletion or disruption of the gene encoding CD5 and/or the TCR receptor alpha chain (TRAC). In further embodiments, the T cell comprises a suicide gene. In non-limiting examples the suicide gene expressed in the CD5*TRAC-tCARTΔCD5ΔTRAC cells encodes a modified Human-Herpes Simplex Virus-1-thymidine kinase (TK) gene fused in-frame to the extracellular and transmembrane domains of the human CD34 eDNA.

In a second embodiment, the disclosure provides an engineered T cell compromising a tCAR that specifically binds CD7 and TCR receptor alpha chain (TRAC), wherein the T cell is deficient in CD7 and TRAC (e.g., CD7*TRAC-tCARTΔCD7ΔTRAC cell). In non-limiting examples the deficiency in CD7 and the TCR receptor alpha chain (TRAC) resulted from (a) modification of CD5 and the TCR receptor alpha chain (TRAC) expressed by the T cell such that the chimeric antigen receptor no longer specifically binds the modified CD7 and the TCR receptor alpha chain (TRAC), (b) modification of the T cell such that expression of the CD7 and the TCR receptor alpha chain (TRAC) is reduced in the T cell by at least 50%, at least 60%, at least 70%, at least 80%, at least 90% or more, or (c) modification of the T cell such that CD7 and the TCR receptor alpha chain (TRAC) is not expressed (e.g., by deletion or disruption of the gene encoding CD7 and/or the TCR receptor alpha chain (TRAC). In further embodiments, the T cell comprises a suicide gene. In non-limiting examples the suicide gene expressed in the CD7*TRAC-tCARTΔCD7ΔTRAC cells encodes a modified Human-Herpes Simplex Virus-1-thymidine kinase (TK) gene fused in-frame to the extracellular and transmembrane domains of the human CD34 eDNA.

In a third embodiment, the disclosure provides an engineered T cell compromising a tCAR that specifically binds CD2 and TCR receptor alpha chain (TRAC), wherein the T cell is deficient in CD2 and TRAC (e.g., CD2*TRAC-tCARTΔCD2ΔTRAC cell). In non-limiting examples the deficiency in CD2 and the TCR receptor alpha chain (TRAC) resulted from (a) modification of CD2 and the TCR receptor alpha chain (TRAC) expressed by the T cell such that the chimeric antigen receptor no longer specifically binds the modified CD2 and the TCR receptor alpha chain (TRAC), (b) modification of the T cell such that expression of the CD7 and the TCR receptor alpha chain (TRAC) is reduced in the T cell by at least 50%, at least 60%, at least 70%, at least 80%, at least 90% or more, or (c) modification of the T cell such that CD2 and the TCR receptor alpha chain (TRAC) is not expressed (e.g., by deletion or disruption of the gene encoding CD2 and/or the TCR receptor alpha chain (TRAC). In further embodiments, the T cell comprises a suicide gene. In non-limiting examples the suicide gene expressed in the CD2*TRAC-tCARTΔCD2ΔTRAC cells encodes a modified Human-Herpes Simplex Virus-1-thymidine kinase (TK) gene fused in-frame to the extracellular and transmembrane domains of the human CD34 eDNA.

In a similar manner, other tandem CAR-T cells can be constructed, and are given below in Table 4-5.

E. Mono iNKT-CAR Cells

In certain embodiments, the disclosure provides an engineered iNKT cell comprising a single CAR, that specifically binds CD7, wherein the iNKT cell is deficient in CD7 (e.g., CD7-iNKT-CARΔCD7 cell). In non-limiting examples, the deficiency in CD7 resulted from (a) modification of CD7 expressed by the iNKT cell such that the chimeric antigen receptors no longer specifically binds the modified CD7, (b) modification of the iNKT cell such that expression of CD7 is reduced in the iNKT cell by at least 50%, at least 60%, at least 70%, at least 80%, at least 90% or more, or (c) modification of the iNKT cell such that CD7 is not expressed (e.g., by deletion or disruption of the gene encoding CD7. In further embodiments, the iNKT cell comprises a suicide gene. In non-limiting examples the suicide gene expressed in the CD7-iNKT-CARΔCD7 cells encodes a modified Human-Herpes Simplex Virus-1-thymidine kinase (TK) gene fused in-frame to the extracellular and transmembrane domains of the human CD34 cDNA.

The CAR for a CD7 specific iNKT-CAR cell can be generated by cloning a commercially synthesized anti-CD7 single chain variable fragment (scFv) into a 3rd generation CAR backbone with CD28 and 4-1BB internal signaling domains. An extracellular hCD34 domain can be added after a P2A peptide to enable both detection of CAR following viral transduction and purification using anti-hCD34 magnetic beads. A similar method can be followed for making CARs specific for other malignant T cell antigens.

In a similar manner, other mono iNKT-CARs can be constructed, and are given below in Table 3 and Table 5.

In some aspects, the present disclosure is directed to a method of treating a cancer in a subject in need thereof comprising administering to the subject a population of mono iNKT CAR cells, and an IL-7 protein fused to a half-life extending moiety, e.g., an Fc region. In some aspects, the present disclosure is directed to a method of treating a cancer in a subject in need thereof comprising administering to the subject a population of mono iNKT-CAR cells, and an IL-7 protein fused to a half-life extending moiety, e.g., an Fc region, wherein the administration results in improved properties, e.g., increased anti-tumor efficacy, improved PK profile, and/or less toxicity, compared to a combination therapy of the mono iNKT-CAR cells and an IL-7 protein not fused to any half-life extending moiety (e.g., not fused to an Fc region) or compared to a monotherapy of the mono iNKT-CAR cells or an IL-7 protein not fused to any half-life extending moiety (e.g., not fused to an Fc region).

F. Dual iNKT-CAR Cells

In certain embodiments, the disclosure provides an engineered iNKT cell comprising a dual CAR (dCAR), i.e., two CARs expressed from a single lentivirus construct, that specifically binds CD7 and CD2, wherein the iNKT cell is deficient in CD7 and CD2 (e.g., CD7×CD2-iNKT-dCARΔCD7ΔCD2 cell). In non-limiting examples, the deficiency in CD7 and CD2 resulted from (a) modification of CD7 and CD2 expressed by the iNKT cell such that the chimeric antigen receptors no longer specifically binds the modified CD7 or CD2, (b) modification of the iNKT cell such that expression of CD7 and CD2 is reduced in the iNKT cell by at least 50%, at least 60%, at least 70%, at least 80%, at least 90% or more, or (c) modification of the iNKT cell such that CD7 and CD2 is not expressed (e.g., by deletion or disruption of the gene encoding CD7 and/or CD2. In further embodiments, the iNKT cell comprises a suicide gene. In non-limiting examples the suicide gene expressed in the CD7*CD2-iNKT-dCARΔCD7ΔCD2 cells encodes a modified Human-Herpes Simplex Virus-1-thymidine kinase (TK) gene fused in-frame to the extracellular and transmembrane domains of the human CD34 cDNA.

In a similar manner, other dual iNKT-CARs can be constructed, and are given below in Tables 4-5.

In some aspects, the present disclosure is directed to a method of treating a cancer in a subject in need thereof comprising administering to the subject a population of dual iNKT CAR cells, and an IL-7 protein fused to a half-life extending moiety, e.g., an Fc region. In some aspects, the present disclosure is directed to a method of treating a cancer in a subject in need thereof comprising administering to the subject a population of dual iNKT-CAR cells, and an IL-7 protein fused to a half-life extending moiety, e.g., an Fc region, wherein the administration results in improved properties, e.g., increased anti-tumor efficacy, improved PK profile, and/or less toxicity, compared to a combination therapy of the dual iNKT-CAR cells and an IL-7 protein not fused to any half-life extending moiety (e.g., not fused to an Fc region) or compared to a monotherapy of the dual iNKT-CAR cells or an IL-7 protein not fused to any half-life extending moiety (e.g., not fused to an Fc region).

G. Tandem iNKT-CAR Cells

In certain embodiments, the disclosure provides an engineered iNKT cell comprising a tandem CAR (tCAR), i.e., two scFv sharing a single intracellular domain, that specifically binds CD7 and CD2, wherein the iNKT cell is deficient in CD7 and CD2 (e.g., CD7×CD2-iNKT-tCARΔCD7ΔCD2 cell). In non-limiting examples, the deficiency in CD7 and CD2 resulted from (a) modification of CD7 and CD2 expressed by the iNKT cell such that the chimeric antigen receptors no longer specifically binds the modified CD7 or CD2, (b) modification of the iNKT cell such that expression of CD7 and CD2 is reduced in the iNKT cell by at least 50%, at least 60%, at least 70%, at least 80%, at least 90% or more, or (c) modification of the iNKT cell such that CD7 and CD2 is not expressed (e.g., by deletion or disruption of the gene encoding CD7 and/or CD2. In further embodiments, the iNKT cell comprises a suicide gene. In non-limiting examples the suicide gene expressed in the CD7*CD2-iNKT-tCARΔCD7ΔCD2 cells encodes a modified Human-Herpes Simplex Virus-1-thymidine kinase (TK) gene fused in-frame to the extracellular and transmembrane domains of the human CD34 cDNA.

A tCAR for a genome-edited, tandem iNKT-CAR cell, i.e., CD7*CD2-iNKT-tCARΔCD7ΔCD2, can be generated by cloning a commercially synthesized anti-CD7 single chain variable fragment (scFv) and an anti-CD2 single chain variable fragment (scFv) into a 3rd generation CAR backbone with CD28 and 4-1BB internal signaling domains. An extracellular hCD34 domain can be added after a P2A peptide to enable both detection of CAR following viral transduction and purification using anti-hCD34 magnetic beads. A similar method can be followed for making tCARs specific for other malignant T cell antigens.

In a similar manner, other tandem iNKT-CARs can be constructed, and are given below in Tables 4-5.

In some aspects, the present disclosure is directed to a method of treating a cancer in a subject in need thereof comprising administering to the subject a population of tandem iNKT CAR cells, and an IL-7 protein fused to a half-life extending moiety, e.g., an Fc region. In some aspects, the present disclosure is directed to a method of treating a cancer in a subject in need thereof comprising administering to the subject a population of tandem iNKT-CAR cells, and an IL-7 protein fused to a half-life extending moiety, e.g., an Fc region, wherein the administration results in improved properties, e.g., increased anti-tumor efficacy, improved PK profile, and/or less toxicity, compared to a combination therapy of the tandem iNKT-CAR cells and an IL-7 protein not fused to any half-life extending moiety (e.g., not fused to an Fc region) or compared to a monotherapy of the tandem iNKT-CAR cells or an IL-7 protein not fused to any half-life extending moiety (e.g., not fused to an Fc region).

### H. Methods for CAR Construction

CARs can be further designed as disclosed in WO2018027036A1, optionally employing variations which will be known to those of skill in the art. Lentiviral vectors and cell lines can be obtained, and guide RNAs designed, validated, and synthesized, as disclosed therein as well as by methods known in the art and from commercial sources.

Engineered CARs can be introduced into T cells or iNKT cells using retroviruses, which efficiently and stably integrate a nucleic acid sequence encoding the chimeric antigen receptor into the target cell genome. Other methods known in the art include, but are not limited to, lentiviral transduction, transposon-based systems, direct RNA transfection, and CRISPR/Cas systems (e.g., type I, type II, or type Ill systems using a suitable Cas protein such Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1 0, Csx16, CsaX, Csx3, Csz1 Csx15, Csf1, Csf2, Csf3, Csf4, and Cu1966, etc.). Zinc finger nucleases (ZFNs) and transcription activator-like effector nucleases (TALENs) can also be used. See, e.g., Shearer R F and Saunders D N, “Experimental design for stable genetic manipulation in mammalian cell lines: lentivirus and alternatives,” Genes Cells 2015 January;20(1):1-10.

### I. Definitions

As used herein, the terms below have the meanings indicated. Other definitions can occur throughout the specification.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range can be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

As used herein, the term “fusion protein” refers to proteins created through the joining of two or more genes that originally coded for separate proteins. Translation of this fusion gene results in a single polypeptide or multiple polypeptides with functional properties derived from each of the original proteins. In some embodiments, the two or more genes can comprise a substitution, a deletion, and/or an addition in its nucleotide sequence.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “composition” as used herein refers to an immunotherapeutic cell population combination with one or more therapeutically acceptable carriers.

The term “deglycosylation” as used herein refers to an Fc region in which sugars are removed enzymatically from an Fc fragment. Additionally, the term “aglycosylation” means that an Fc fragment is produced in an unglycosylated form by a prokaryote, and preferably in E. coli.

As used herein, the term “dimer” is an oligomer consisting of two monomers joined by bonds that can be either strong or weak, covalent, or intermolecular. The term “homodimer” is used when the two molecules are identical, e.g. A-A, and “heterodimer” when they are not, e.g. A-B.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “effector function” refers to a specialized function of a differentiated cell. An effector function of a T cell, for example, can be cytolytic activity or helper activity including the secretion of cytokines. An effector function in a naïve, memory, or memory-type T cell can also include antigen-dependent proliferation.

The term “fratricide” as used herein means a process which occurs when a CAR-T cell or iNKT-CAR cell becomes the target of, and is killed by, another CAR-T cell or iNKT-CAR cell comprising the same chimeric antigen receptor as the target of CAR-T or iNKT-CAR cell, because the targeted cell expresses the antigen specifically recognized by the chimeric antigen receptor on both cells. CAR-T cells or iNKT-CARs comprising a chimeric antigen receptor which are deficient in an antigen to which the chimeric antigen receptor specifically binds will be “fratricide-resistant.”

As used herein, the term “gene expression” or “expression” of an IL-7 protein is understood to refer to transcription of a DNA sequence, translation of an mRNA transcript, and secretion of a fusion protein product, or an antibody, or an antibody fragment thereof.

The term “genome-edited” as used herein means having a gene added, deleted, or modified to be non-functional. Thus, in certain embodiments, a “gene-edited T cell” or a “gene-edited iNKT cell” is a T cell or iNKT cell that has had a gene such as a CAR recognizing at least one antigen added; and/or has had a gene such as the gene(s) to the antigen(s) that are recognized by the CAR deleted.

A “healthy donor,” as used herein, is one who does not have a hematologic malignancy (e.g., a T-cell malignancy).

As used herein, the term “host cell” refers to a prokaryotic cell and/or a eukaryotic cell into which a recombinant expression vector can be introduced.

Unless otherwise specified, the terms “protein”, “polypeptide”, and “peptide” can be used as an interchangeable concept.

The term “immune checkpoint inhibitor” refers to a type of drug that blocks certain proteins made by some types of immune system cells, such as T cells, and some cancer cells.

The term “immune effector cell,” as used herein, are cells that are actively involved in the destruction of tumor cells, e.g., possess anti-tumor activity. These cells can include, but are not limited to, natural killer (NK) cells, cytotoxic T cells, and memory T cells.

The term “chimeric antigen receptor (CAR)-bearing immune effector cells are immune effector cells that express a chimeric antigen receptor. These cells can include, but are not limited to, CAR-T cells or CAR-bearing iNKT cells (iNKT-CAR).

The term “CAR-T cell” means a CAR-T cell that expresses a chimeric antigen receptor.

A dual CAR-T cell (equivalently, dCAR-T) is a CAR-T cell that expresses two distinct chimeric antigen receptor polypeptides with affinity to different target antigens expressed within the same effector cell, wherein each CAR functions independently. The CAR can be expressed from a single polynucleotide sequence or multiple polynucleotide sequences.

A tandem CAR-T cell (equivalently, tCAR-T) is a CAR-T cell with a single chimeric antigen polypeptide containing two distinct antigen recognition domains with affinity to different targets, wherein the antigen recognition domains are linked through a peptide linker and share common costimulatory domain(s), and wherein binding of either antigen recognition domain will signal though a common costimulatory domains(s) and signaling domain.

The term CAR-iNKT cell (equivalently, iNKT-CAR) means an iNKT cell that expresses a chimeric antigen receptor.

A dual iNKT-CAR cell (equivalently, iNKT-dCAR) is an iNKT-CAR cell that expresses two distinct chimeric antigen receptor polypeptides with affinity to different target antigens expressed within the same effector cell, wherein each CAR functions independently. The CAR can be expressed from a single polynucleotide sequence or multiple polynucleotide sequences.

A tandem iNKT-CAR cell (equivalently, iNKT-tCAR) is an iNKT-CAR cell with a single chimeric antigen polypeptide containing two distinct antigen recognition domains with affinity to different targets, wherein the antigen recognition domains are linked through a peptide linker and share common costimulatory domain(s), and wherein binding of either antigen recognition domain will signal though a common costimulatory domains(s) and signaling domain.

As used herein, the term “modified” refers to a polypeptide or protein having the same or similar sequence and activity to IL-7.

The term “patient” is generally synonymous with the term “subject” and includes all mammals including humans.

As used herein, the term “signal sequence,” or equivalently, “signal peptide,” refers to a fragment directing the secretion of a biologically active molecule drug and a fusion protein, and it is cut off after being translated in a host cell. The signal sequence as used herein is a polynucleotide encoding an amino acid sequence initiating the movement of the protein penetrating the endoplasmic reticulum (ER) membrane. Useful signal sequences include an antibody light chain signal sequence, e.g., antibody 14.18 (Gillies et al., J. Immunol. Meth 1989. 125:191-202), an antibody heavy chain signal sequence, e.g., MOPC141 an antibody heavy chain signal sequence (Sakano et al., Nature, 1980.286: 676-683), and other signal sequences know in the art (e.g., see Watson et al., Nucleic Acid Research, 1984.12:5145-5164). The characteristics of signal peptides are well known in the art, and the signal peptides conventionally having 16 to 30 amino acids, but they can include more or less number of amino acid residues. Conventional signal peptides consist of three regions of the basic N-terminal region, a central hydrophobic region, and a more polar C-terminal region.

The term “therapeutically acceptable” refers to substances which are suitable for use in contact with the tissues of patients without undue toxicity, irritation, and allergic response, are commensurate with a reasonable benefit/risk ratio, and/or are effective for their intended use.

The term “therapeutically effective” is intended to qualify the amount of active ingredients used in the treatment of a disease or disorder or on the effecting of a clinical endpoint.

As used herein, the terms “transduced”, “transformed”, and “transfected” refer to the introduction of a nucleic acid (e.g., a vector) into a cell using a technology known in the art.

As used herein, the term “vector” is understood as a nucleic acid means which includes a nucleotide sequence that can be introduced into a host cell to be recombined and inserted into the genome of the host cell, or spontaneously replicated as an episome. The vector can include linear nucleic acids, plasmids, phagemids, cosmids, RNA vectors, virus vectors, and analogs thereof. Examples of the virus vectors can include retroviruses, adenoviruses, and adeno-associated viruses, but are not limited thereto.

### EXAMPLES

The invention is further illustrated by the following examples. In the Examples below, NT-I7 is a protein having a primary sequence chosen from SEQ ID NO.s 21-25:

SEQ ID NO: 21
MDCDIEGKDGKQYESVLMVSIDQLLDSMKEIGSNCLNNEFNFFKRHIC
DANKEGMFLFRAARKLRQFLKMNSTGDFDLHLLKVSEGTTILLNCTGQ
VKGRKPAALGEAQPTKSLEENKSLKEQKKLNDLCFLKRLLQEIKTCWN
KILMGTKEHRNTGRGGEEKKKEKEKEEQEERETKTPECPSHTQPLGVF
LFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNG
QPENNYKTTPPVLDSDGSFFLYSRLTVDKSRWQEGNVFSCSVMHEALH
NHYTQKSLSLSLGK
SEQ ID NO: 22
MMDCDIEGKDGKQYESVLMVSIDQLLDSMKEIGSNCLNNEFNFFKRHI
CDANKEGMFLFRAARKLRQFLKMNSTGDFDLHLLKVSEGTTILLNCTG
QVKGRKPAALGEAQPTKSLEENKSLKEQKKLNDLCFLKRLLQEIKTCW
NKILMGTKEHRNTGRGGEEKKKEKEKEEQEERETKTPECPSHTQPLGV
FLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESN
GQPENNYKTTPPVLDSDGSFFLYSRLTVDKSRWQEGNVFSCSVMHEAL
HNHYTQKSLSLSLGK
SEQ ID NO: 23
MMMDCDIEGKDGKQYESVLMVSIDQLLDSMKEIGSNCLNNEFNFFKRH
ICDANKEGMFLFRAARKLRQFLKMNSTGDFDLHLLKVSEGTTILLNCT
GQVKGRKPAALGEAQPTKSLEENKSLKEQKKLNDLCFLKRLLQEIKTC
WNKILMGTKEHRNTGRGGEEKKKEKEKEEQEERETKTPECPSHTQPLG
VFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKT
ISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEA
LHNHYTQKSLSLSLGK
SEQ ID NO: 24
MGMDCDIEGKDGKQYESVLMVSIDQLLDSMKEIGSNCLNNEFNFFKRH
ICDANKEGMFLFRAARKLRQFLKMNSTGDFDLHLLKVSEGTTILLNCT
GQVKGRKPAALGEAQPTKSLEENKSLKEQKKLNDLCFLKRLLQEIKTC
WNKILMGTKEHRNTGRGGEEKKKEKEKEEQEERETKTPECPSHTQPLG
VFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKT
ISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEA
LHNHYTQKSLSLSLGK
SEQ ID NO: 25
MMMMDCDIEGKDGKQYESVLMVSIDQLLDSMKEIGSNCLNNEFNFFKR
HICDANKEGMFLFRAARKLRQFLKMNSTGDFDLHLLKVSEGTTILLNC
TGQVKGRKPAALGEAQPTKSLEENKSLKEQKKLNDLCFLKRLLQEIKT
CWNKILMGTKEHRNTGRGGEEKKKEKEKEEQEERETKTPECPSHTQPL
GVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEK
TISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHE
ALHNHYTQKSLSLSLGK

### Example 1—UCART19 with Administration of NT-I7—Experimental Design

The following steps disclosed herein can be taken to provide the gene-edited CAR-T, e.g. UCART19 and to assay the effect of NT-I7, on CAR-T expansion, persistence, and anti-tumor activity. As those of skill in the art will recognize, certain of the steps can be conducted sequentially or out of the order listed below, though perhaps leading to different efficiency.

Step 1. Peripheral blood mononuclear cells (PBMCs) are harvested from one or more healthy donors.

Step 2. T cells were then isolated/purified from donor PBMCs using magnetic selection with a labelled antibody-coated magnetic beads (e.g., Miltenyi Biotech). Other purification techniques are known in the art and could be used.

Step 3. T cells were then activated using anti-CD3 and anti-CD28 antibodies. In the case of TCR deletion, the TCR is composed of proteins expressed prior to genome editing in sufficient quantities to allow for activation of the TCR until loss of these protein occur.

Step 4. If a CAR targeting one or more antigens is to be transduced into the cell, the antigen that is the target of the CAR can be deleted from the cell surface or its expression suppressed to prevent subsequent fratricide. In this example, the CAR targeted the CD19 antigen. Target deletion can be accomplished by electroporation with Cas9 mRNA and guide RNA (gRNA) against the target(s). The deletion of TRAC prevents Graft versus Host Disease (GVHD) from occurring in these genome-edited CAR-T cells. Other techniques, however, could be used to suppress expression of the target(s). These include other genome editing techniques such as TALENs, ZFNs, RNA interference, and eliciting of internal binding of the antigen to prevent cell surface expression. Examples of gRNA sequences for targeted genes are listed in Table 1.

TABLE 1
Guide RNA sequences

| Target | |
|---|---|
| gene | gRNA sequence |
| TRACg | 5′_2′OMe(G(ps)A(ps)G(ps))AAUCAAAAUCGGUG |
| | AAUGUUUUAGAGCUAGAAAUAGCAAGUUAAAAUAAGGCU |
| | AGUCCGUUAUCAACUUGAAAAAGUGGCACCGAGUCGGUG |
| | C2′OMe(U(ps)U(ps)U(ps)U3′ |

RNA; (ps) indicate phosphorothioate.
Underlined bases denote target sequence.

Step 5. T cells were then transduced with a CAR targeted to (i.e., that recognizes) one or more antigen or protein targets, for example with a lentivirus containing a CAR construct, e.g., CD19. Any other suitable method of transduction/transfection can be used, for example transfection using DNA-integrating viral or non-viral vectors containing transposable elements, or transient expressing of non-DNA integrating polynucleotides, such as mRNA, or insertion of CAR polynucleotide into site of nuclease activity using homologous or non-homologous recombination.

Step 6. The UCART19 population was expanded by removal of CD3/CD28 stimulation. This can continue for one week, two weeks or several weeks.

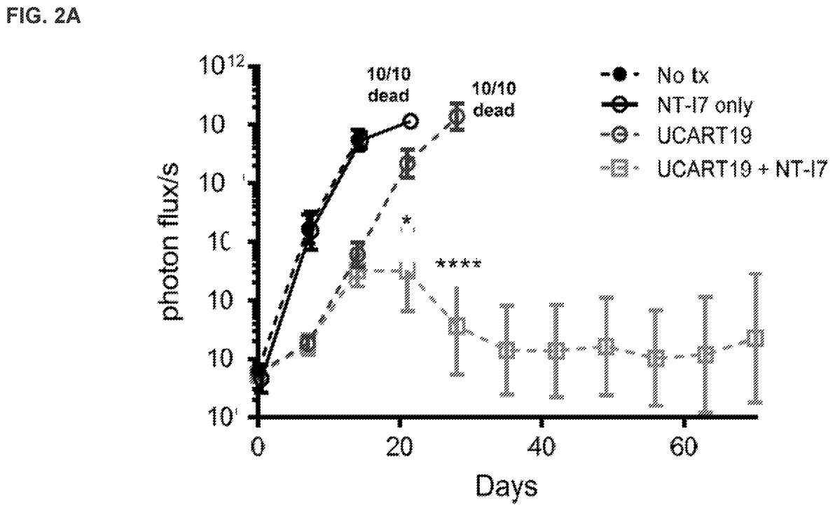

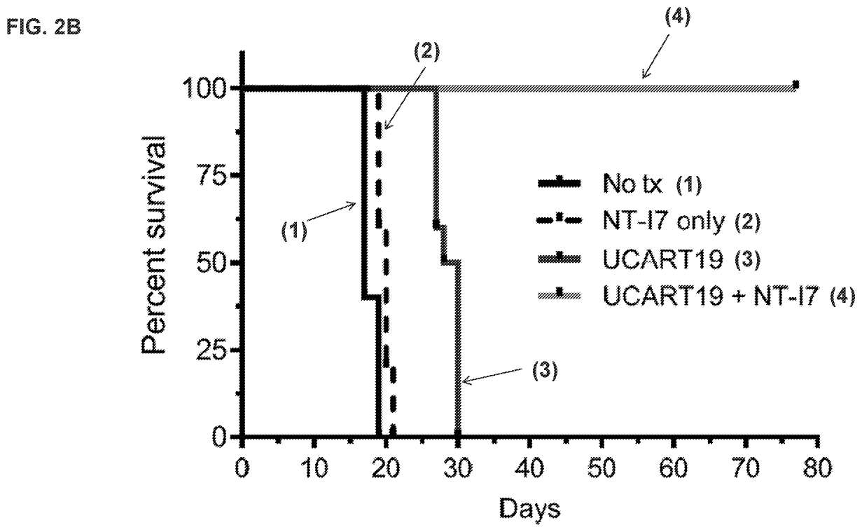

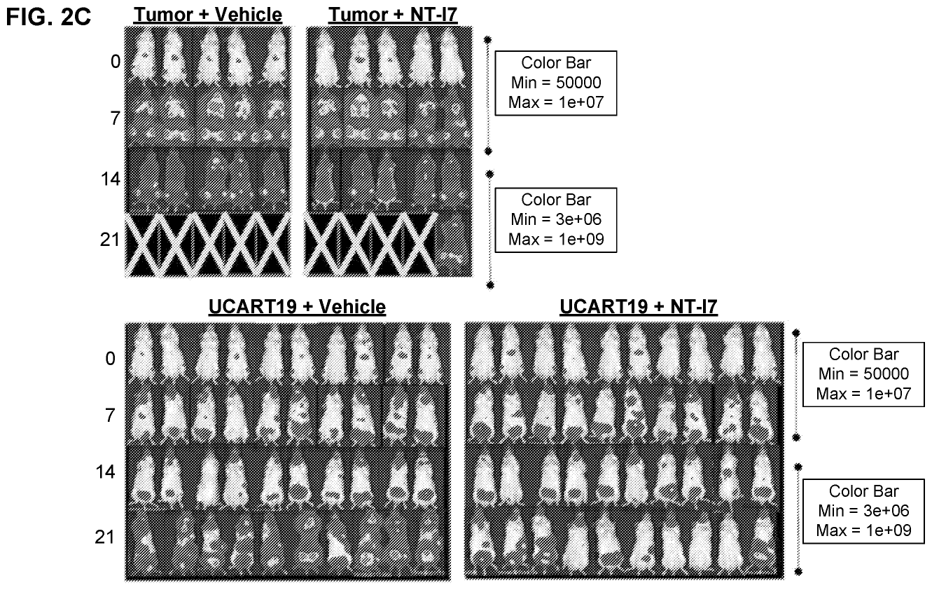

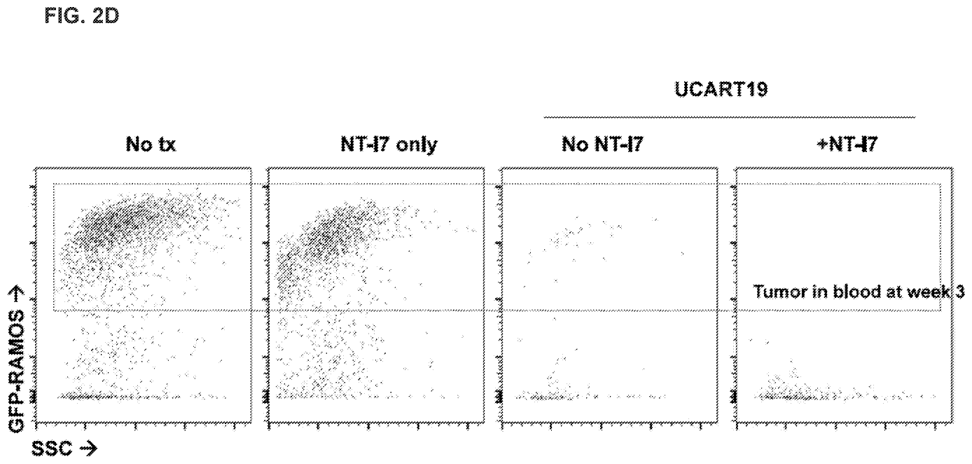

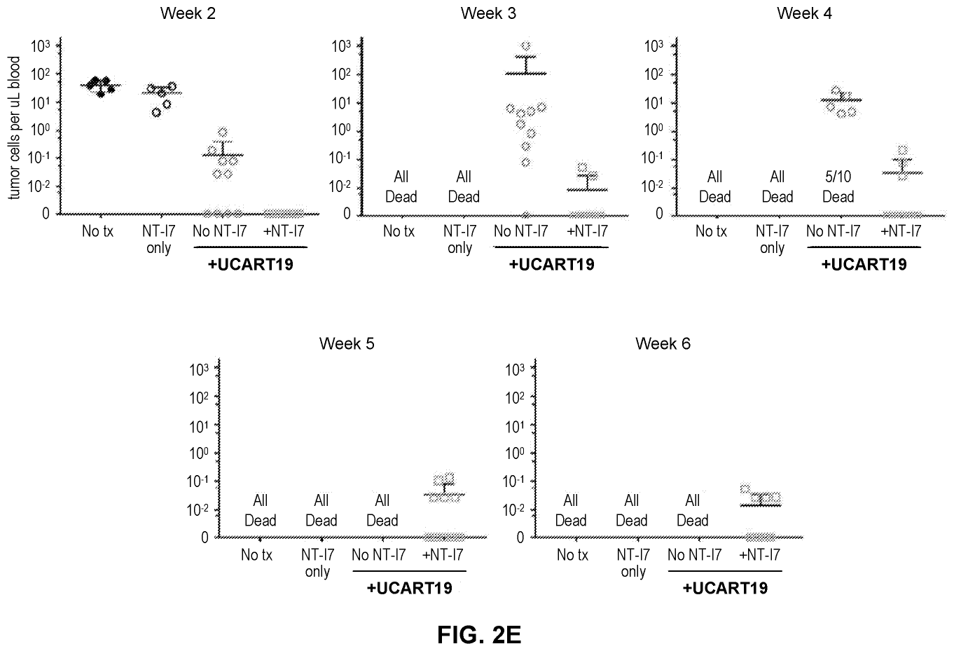

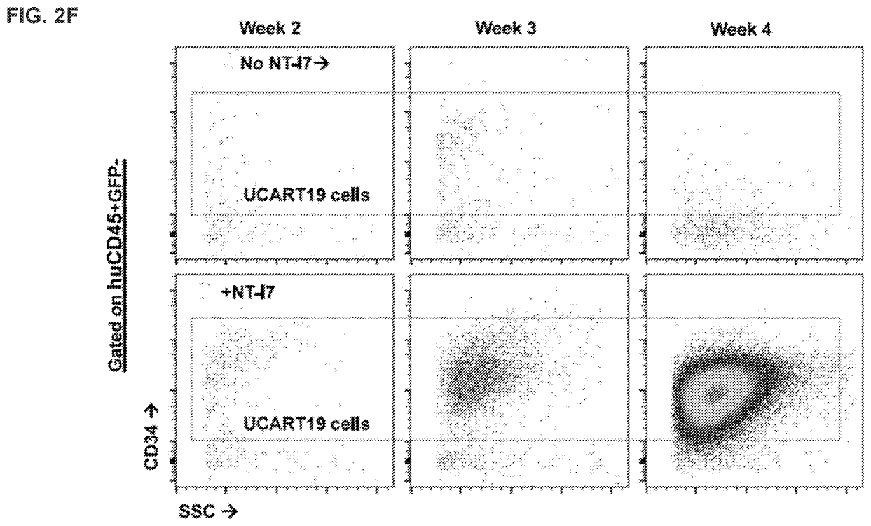

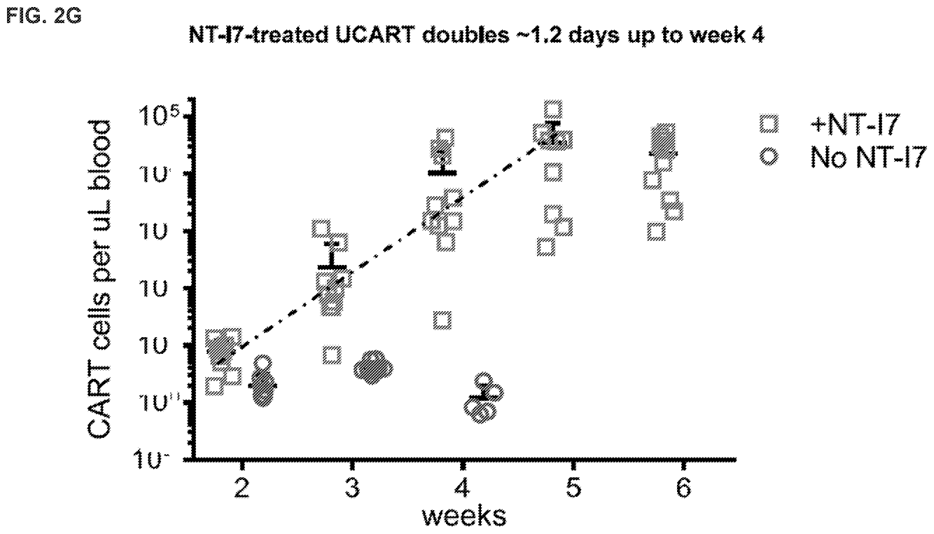

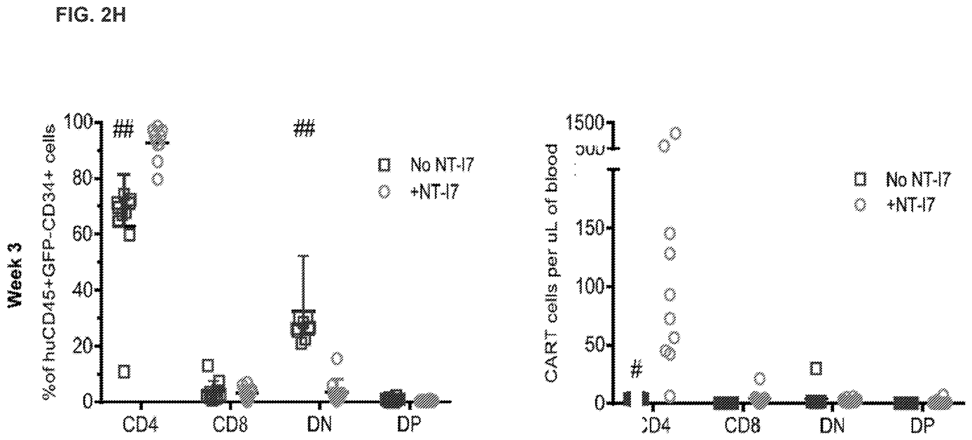

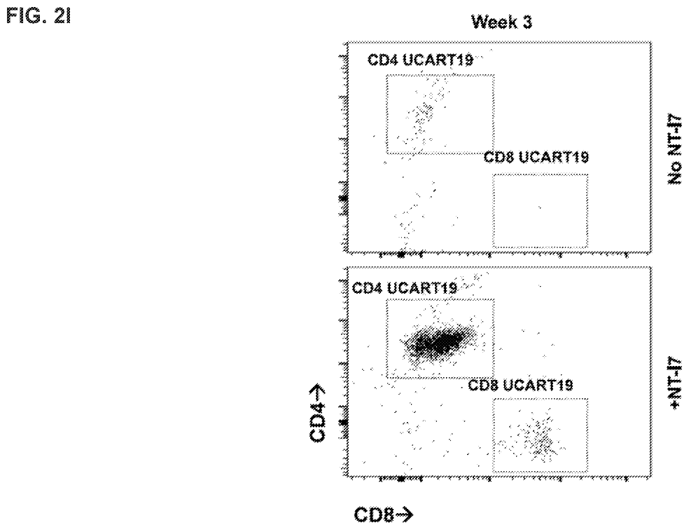

Step 7. Tumor burden and UCART19 kinetics was assessed in triple transgenic NSG-SGM3 (NSGS) mice while monitoring survival, weight, and xenogeneic GVHD (FIG. 2B). NSG-SGM3 (NSGS) mice express human IL3, GM-CSF, and SCF and combine the features of the highly immunodeficient NOD SCID gamma (NSG) mouse with cytokines to support the stable engraftment of myeloid lineages and regulatory T cell populations. In this experiment, NSG-SGM3 mice were injected with 5×10⁵ of a GFP-expressing B-cell ALL cell line (Ramos^{CBR-GFP}) four days prior to the administration about 2×10⁶ to about 3×10⁷ UCART19 cells/mouse (Day 0;). On Day 1, mice (N=10/group) received either no treatment (tx), NT-I7 (10 mg/kg), UCART19, or UCART19+NT-I7. In experimental groups that received NT-I7, the protocol indicated that NT-I7 was injected every 2 weeks thereafter.

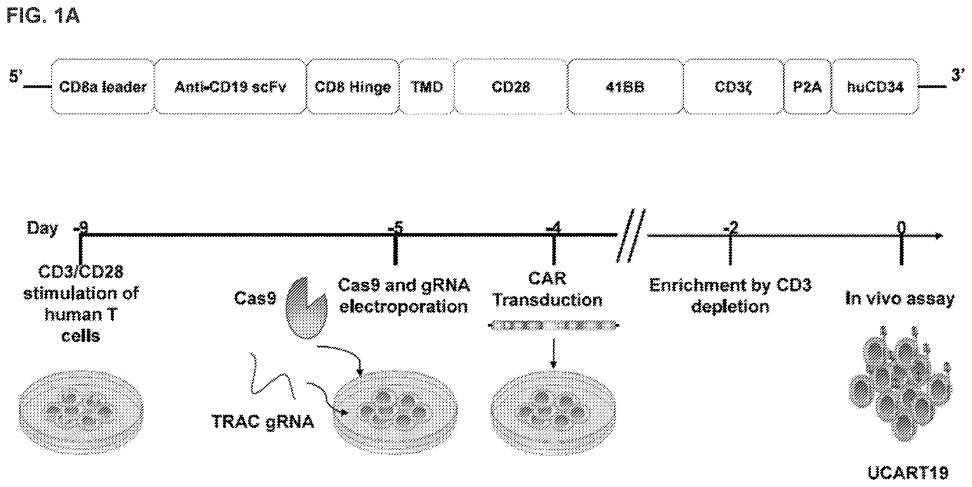

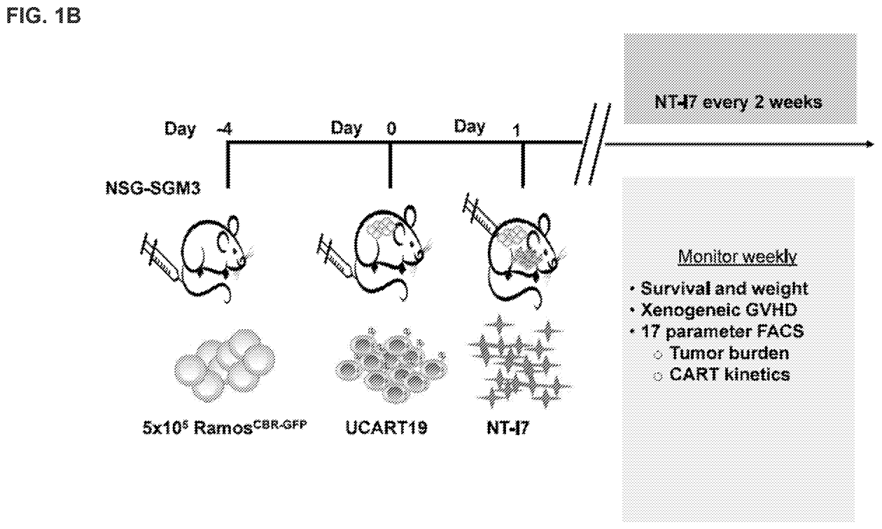

These steps are shown as flow diagrams in FIG. 1A and FIG. 1B. Those of skill in the art will appreciate that some flexibility is possible in the time frames specified in FIG. 1A.

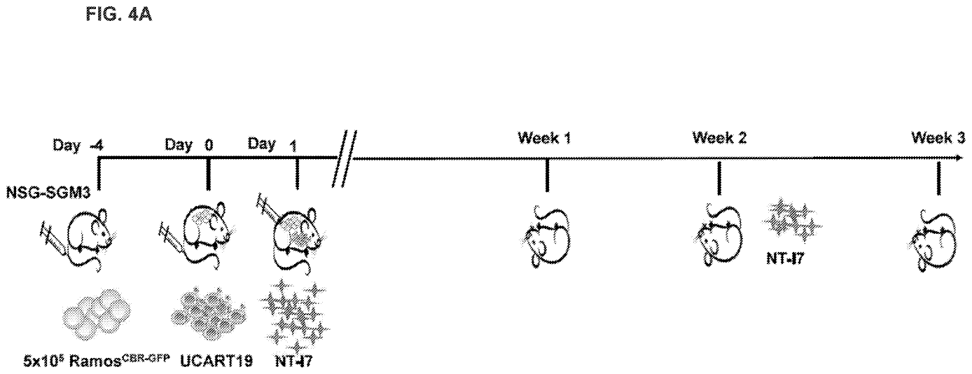

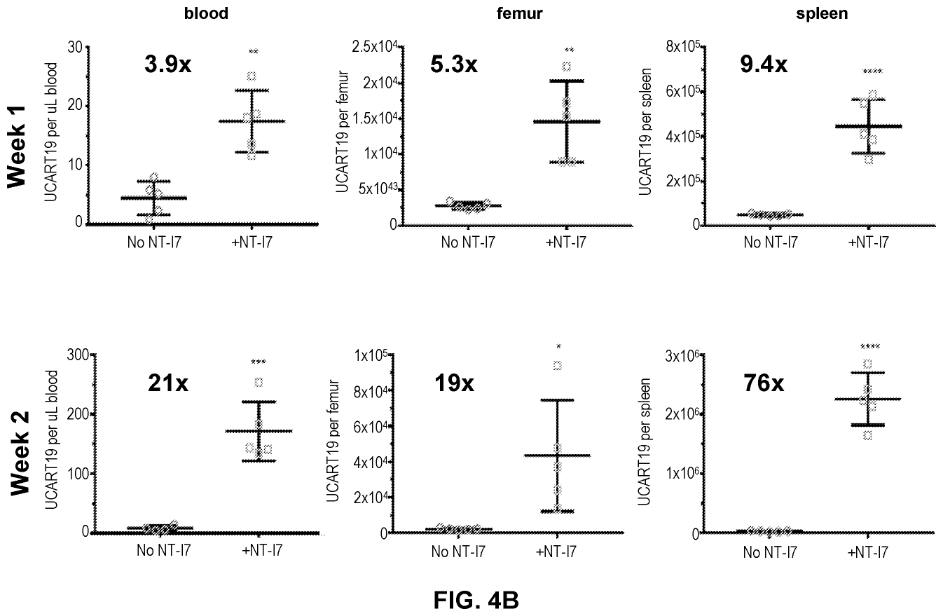

Experimental results showed that the administration of UCART19 cells and NT-I7 effectively kill the B-cell ALL cell line Ramos and indefinitely prolong survival (FIG. 2A, FIG. 2B, and FIG. 2C). Percent survival was assessed and all mice (10/10-dead) were dead in the two experimental groups that received no treatment (Day 19) or NT-I7 only (Day 21) (FIG. 2B). In the experimental group that received UCART19 cells, all mice (10/10-dead) were dead by Day 30 (FIG. 2B). In the experimental group that received UCART19+NT-I7 all mice (10/10-alive) were alive at Day 75 and tumor burden was shown to be significantly reduced as measured in a bioluminescent imaging assay (FIG. 2B, FIG. 2C). In addition, all mice (10/10) receiving UCART19+NT-I7 treatment did not show symptoms of GVHD. Furthermore, the data showed tumor elimination in mice receiving UCART19+NT-I7 treatment. This was assessed by FACS analysis at week 3 (FIG. 2D) and by measuring tumor cells/μL of blood at week 2, week 3, week 4, week 5, and week 6 in experimental groups receiving no treatment, NT-I7 only, UCART19 only, and NT-I7+ UCART19 treatment (FIG. 2E). The data shown that NT-I7 induces a significant expansion of CD4+ when compared to control populations with NT-I7 administration. This was assessed by FACS and measuring CAR-T cells/μL of blood (FIG. 2F-FIG. 2H). Similar results were observed in the spleen and the bone marrow (FIGS. 4A and 4B).

### Example 2—UCART2 with Administration of NT-I7 for the Treatment of T Cell Hematologic Malignancies

The following steps disclosed herein can be taken to provide the gene-edited CAR-T, e.g. UCART2, and to assay the effect of NT-I7 on CAR-T expansion, persistence, and anti-tumor activity. As those of skill in the art will recognize, certain of the steps can be conducted sequentially or out of the order listed below, though perhaps leading to different efficiency.

Step 1. Peripheral blood mononuclear cells (PBMCs) are harvested from one or more healthy donors.

Step 2. T cells were then isolated/purified from donor PBMCs using magnetic selection with a labelled antibody-coated magnetic beads (e.g., Miltenyi Biotech). Other purification techniques are known in the art and could be used.

Step 3. T cells were then activated using anti-CD3 and anti-CD28 antibodies. In the case of TCR deletion, the TCR is composed of proteins expressed prior to genome editing in sufficient quantities to allow for activation of the TCR until loss of these protein occur.

Step 4. If a CAR targeting one or more antigens is to be transduced into the cell, the antigen that is the target of the CAR can be deleted from the cell surface or its expression suppressed to prevent subsequent fratricide. In this example, the CAR targeted the CD2 antigen. Target deletion can be accomplished by electroporation with Cas9 mRNA and guide RNA (gRNA) against the target(s). In this example, CD2 and TRAC were targeted for deletion. The deletion of TRAC prevented Graft versus Host Disease (GVHD) from occurring in these genome-edited CAR-T cells. Other techniques, however, could be used to suppress expression of the target(s). These include other genome editing techniques such as TALENs, ZFNs, RNA interference, and eliciting of internal binding of the antigen to prevent cell surface expression. Examples of gRNA sequences for targeted genes are listed in Table 2.

TABLE 2
Guide RNA sequences

| Target | |
|---|---|
| gene | gRNA sequence |
| TRACg | 5′_2′OMe(G(ps)A(ps)G(ps))AAUCAAAAUCGGUG |
| | AAUGUUUUAGAGCUAGAAAUAGCAAGUUAAAAUAAGGCU |
| | AGUCCGUUAUCAACUUGAAAAAGUGGCACCGAGUCGGUG |
| | C2′OMe(U(ps)U(ps)U(ps)U3′ |
| CD2 | 5′_2′OMe(G(ps)A(ps)C(ps))CAAUCUGACAUGCU |
| | GCAGUUUUAGAGCUAGAAAUAGCAAGUUAAAAUAAGGCU |
| | AGUCCGUUAUCAACUUGAAAAAGUGGCACCGAGUCGGUG |
| | C2′OMe(U(ps)U(ps)U(ps)U_3′ |

RNA; (ps) indicate phosphorothioate.
Underlined bases denote target sequence.

Step 5. T cells can then be transduced with a CAR targeted to (i.e., that recognizes) one or more antigen or protein targets, for example with a lentivirus containing a CAR construct, e.g., CD2. Any other suitable method of transduction/transfection can be used, for example transfection using DNA-integrating viral or non-viral vectors containing transposable elements, or transient expressing of non-DNA integrating polynucleotides, such as mRNA, or insertion of CAR polynucleotide into site of nuclease activity using homologous or non-homologous recombination.

Step 6. The UCART2 population was expanded by removal of CD3/CD28 stimulation. This can continue for one week, two weeks or several weeks.

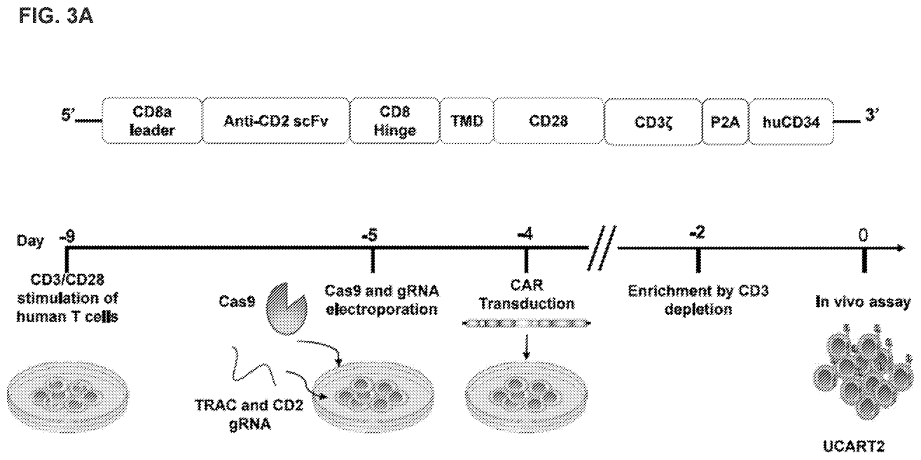

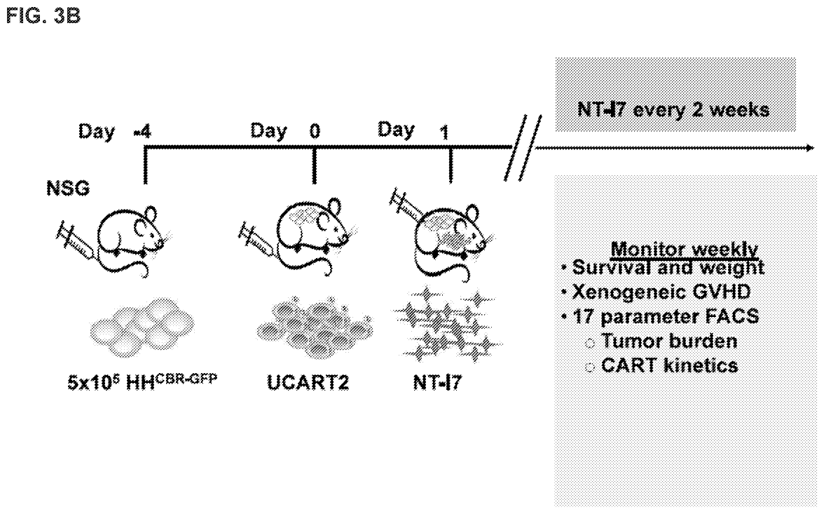

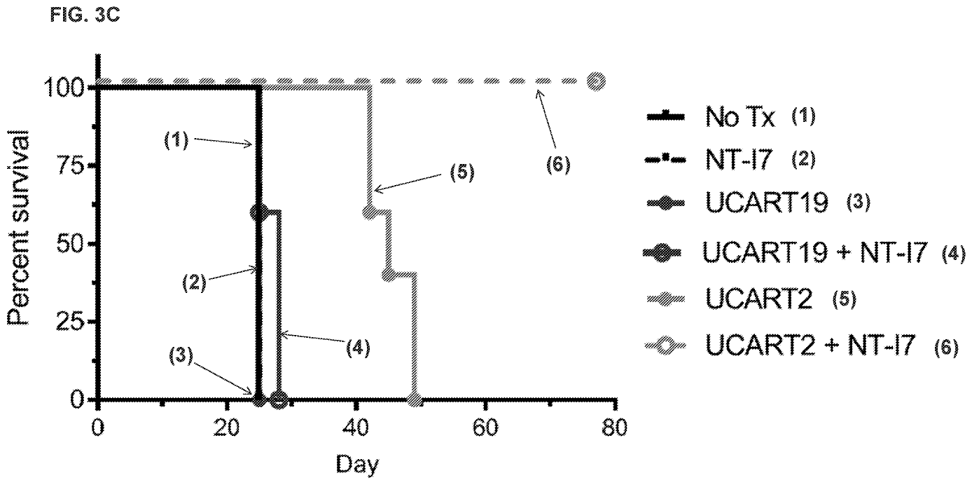

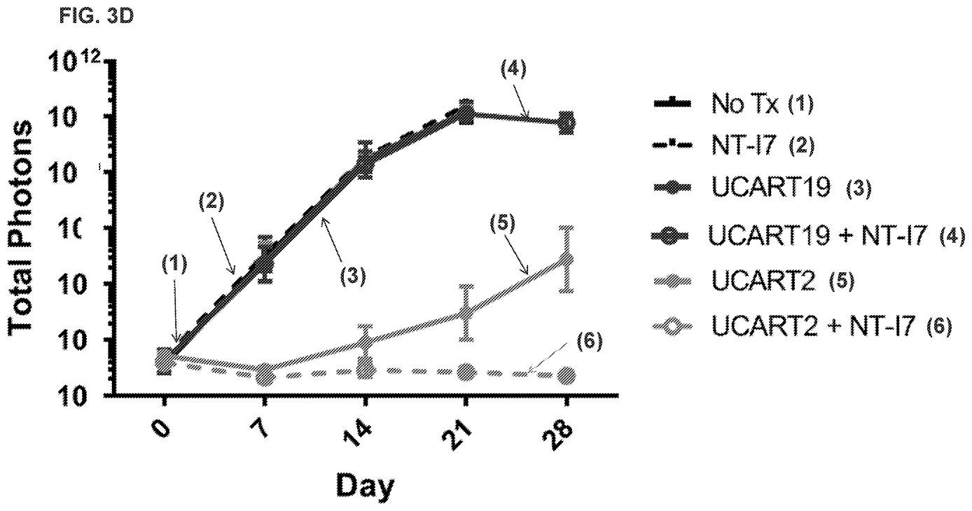

Step 7. Tumor burden and UCART2 kinetics was assessed in triple transgenic NSG-SGM3 (NSGS) mice while monitoring survival, weight, and xenogeneic GVHD (FIG. 3B). NSG-SGM3 (NSGS) mice express human IL3, GM-CSF, and SCF and combine the features of the highly immunodeficient NOD SCID gamma (NSG) mouse with cytokines to support the stable engraftment of myeloid lineages and regulatory T cell populations. In this experiment, NSG-SGM3 mice were injected with 5×10⁵ of a GFP-expressing B-cell ALL cell line (HH^{CBR-GFP}) four days prior to the administration of 3×10⁷ UCART19 cells/mouse (Day 0;). On Day 1, mice (N=10/group) received either no treatment (tx), NT-I7, UCART19, UCART19+NT-I7, UCART2, or UCART2+NT-I7. In the experimental groups which received NT-I7, the protocol indicated that NT-17 was injected every 2 weeks thereafter.

These steps are shown as flow diagrams in FIG. 3A and FIG. 3B. Those of skill in the art will appreciate that some flexibility is possible in the time frames specified in FIG. 3A.

Experimental results showed that the administration of UCART2 cells and NT-I7 effectively kill the T-cell malignant cell line HH and indefinitely prolong survival (FIG. 3C and FIG. 3D). Percent survival was assessed, and the data shows that NT-I7 enhanced survival and significantly reduces the tumor burden in mice receiving UCART2 (FIG. 3C and FIG. 3D).

### Example 3—Tumor Burden and CAR-T Profiles in Different Organs

Tumor and CAR-T Profiles will be evaluated in different organs other than blood using NSG-SGM3 mice injected with 5×10⁵ of a GFP-expressing B-cell ALL cell line (Ramos CBR-GFP). Multi-tissue UCART kinetics, tumor killing kinetics, tumor immunophenotyping, and transcriptional profiling will be assessed in four tested groups: 1) No treatment; 2) NT-I7 only; 3) UCART19 only; and 4) UCART19+NT-I7. An (n=5) will be sacrificed for each group at each time point and four time points will be tested (week 1, week 2, week 3, and week 4). It is expected that CAR-immune effector cell therapy such as UCART19, alone and in combination with NT-I7 will be effective in reducing or eliminating B-ALL. FACS experiments will be performed on marrow, spleen, and blood. Soft tissue will be dissected for tumor evidence. Splenic CD45+CD34+ cells will be assayed for RNA at week 2 and week 3. Splenic GFP+ will be assayed for RNA at week 2 and week 3.

### Example 4—UCART19 and NT-17 in PDX Model of B-ALL

These experiments will be aimed to test efficacy of UCART19 against primary human tumors in a mouse model. The experimental design will emulate that of Example 1. UCART19 will target patient primary B-ALL in vivo. It is expected that CAR-immune effector cell therapy such as UCART19 cells, alone and in combination with NT-I7 will be effective in reducing or eliminating B-ALL.

### Example 5—Efficacy of NT-17 in Solid Tumor Model of B-ALL

NSG-SGM3 mice will be subcutaneous injected (k=hind leg) with 5×10⁵ of a GFP-expressing B-cell ALL cell line (Ramos^{CBR-GFP}) In these mice, survival is monitored and tumor burden (BLI, FACS), UCART kinetics, tumor killing kinetics, T cell and tumor immunophenotyping, recipient hematopoietic profiling, and multiplex cytokine profiling of plasma will be assessed in four tested groups: 1) No treatment (n=5); 2) NT-I7 only (n=5); 3) UCART19 only (n=10); and 4) UCART19+NT-I7 (n=10). Mice will be sacrificed for each group at each time point and four time points will be tested (week 1, week 2, week 3, and week 4). It is expected that CAR-immune effector cell therapy such as UCART19 cells, alone and in combination with NT-I7 will be effective in reducing or eliminating B-ALL.

### Example 6—NT-I7 for the Expansion of CAR-T In Vitro

IL-7 proteins such as NT-I7, could help expand CAR-T during production prior to injection into mice. The deletion of TRAC in UCART limits the expansion of CAR-T due to the lack of a functional TCR. NT-I7, modified IL-7 proteins, can be used to expand T cells in absence of TCR signaling to increase yield. The experiments will assess: 1) the kinetics of CAR-T expansion using INCUCYTE® S3 in presence and absence of CAR-T; 2) CAR-T phenotype of NT-I7 expanded CAR-T cells in vitro; and efficacy of NT-I7 expanded CAR-T cells in vitro (Cr-release assay) and in vivo. It is expected that NT-I7 and/or other native and/or modified IL-7 proteins will be effective in expanding CAR-T cells in vitro, and would be similarly effective in expanding other CAR-bearing immune effector cells such as CART-iNKT cells.

### Example 7—Effect OF NT-I7 on Solid Tumor Model of Pancreatic Cancer Using Mesothelin CAR-T

NSG-SGM3 mice can be subcutaneously injected in the flank with a pancreatic adenocarcinoma cell line (CAPAN-2GFP). In these mice, survival will be monitored and tumor burden (BLI, FACS), UCART kinetics, tumor killing kinetics, T cell and tumor immunophenotyping, recipient hematopoietic profiling, and multiplex cytokine profiling of plasma will be assessed in four tested groups: 1) No treatment (n=5); 2) NT-I7 only (n=5); 3) UCART-Meso only (n=10); and 4) UCART-Meso+NT-I7 (n=10). Mice will be sacrificed for each group at each time point and four time points will be tested (week 1, week 2, week 3, and week 4). It is expected that CAR-immune effector cell therapy such as UCART-Meso cells in combination with NT-I7 will be effective in reducing or eliminating pancreatic adenocarcinoma.

### Example 8—Effect of NT-I7 on PDX Model of Breast Cancer Using UCAR-T Targeting Mesothelin

NSG-SGM3 mice will be subcutaneous injected in the flank with a patient-derived breast cancer. In these mice, survival will be monitored and tumor burden (BLI, FACS), UCART kinetics, tumor killing kinetics, T cell and tumor immunophenotyping, recipient hematopoietic profiling, and multiplex cytokine profiling of plasma will be assessed in four tested groups: 1) No treatment (n=5); 2) NT-I7 only (n=5); 3) UCART-Meso only (n=10); and 4) UCART-Meso+NT-I7 (n=10). Mice will be sacrificed for each group at each time point and four time points will be tested (week 1, week 2, week 3, and week 4). It is expected that CAR-immune effector cell therapy such as UCART-Meso cells in combination with NT-I7 will be effective in reducing or eliminating breast cancer.

### Example 9—Efficacy of NT-I7 Using Alternative Effects Cells (CAR-iNKT)—Solid Tumor Model of B-ALL

It is expected that NT-I7 will be useful for other effector cell populations such as iNKT. NSG-SGM3 mice will be injected with 5×10⁵ of a GFP-expressing B-cell ALL cell line (Ramos CBR-GFP). In these mice, survival will be monitored and tumor burden (BLI, FACS), UCART kinetics, tumor killing kinetics, T cell and tumor immunophenotyping, recipient hematopoietic profiling, and multiplex cytokine profiling of plasma will be assessed in four tested groups: 1) No treatment (n=5); 2) NT-I7 only (n=5); 3) UCART19 only (n=10); and 4) UCART19+NT-I7 (n=10). Mice will be sacrificed for each group at each time point and four time points will be tested (week 1, week 2, week 3, and week 4). It is expected that CAR-immune effector cell therapy such as UCART19 cells, alone and in combination with NT-I7 will be effective in reducing or eliminating B-ALL.

The foregoing Examples in which CAR-T cells are combined with native and/or modified IL-7 proteins can be performed with other CAR-bearing immune effector cells, such as dCAR-Ts, tCAR-Ts, and CAR-iNKTs. Similar gains in expansion, maintenance, tumor infiltration, and the like are expected to be observed, yielding similar benefits in the reduction or elimination of targeted cancerous cells and tumors, and treatment of cancers.

### Example 10—Construction of Mono CAR-T and Mono iNKT-CARs

TABLE 3

| | Antigen Target | |
|---|---|---|
| | of a mono CAR-T | Antigen |
| | or a mono | Deletion/ |
| Example | iNKT-CAR | Suppression |
| 1 | CD2 | — |
| 2 | CD3ε | — |
| 3 | CD4 | — |
| 4 | CD5 | — |
| 5 | CD7 | — |
| 6 | TRAC | — |
| 7 | TCRβ | — |
| 8 | CD2 | CD2 |
| 9 | CD3ε | CD3ε |
| 10 | CD4 | CD4 |
| 11 | CD5 | CD5 |
| 12 | CD7 | CD7 |
| 13 | TRAC | TRAC |
| 14 | TCRβ | TCRβ |

### Example 11—Tandem and Dual CAR-T and iNKT-CARs

Additional examples of tandem and dual CAR-T and iNKT-CARs are provided below, with and without deletion or suppression of one or more surface proteins that is/are the antigen targets of the CARs. In general, examples with deletion or suppression of more antigens will be more likely to have the benefit of greater fratricide resistance. It should be further noted that the order in which the antigens (scFV) are oriented in the tandem CARs set forth below in Table 4 is not meant to be limiting and includes tandem CAR-T and iNKT-CARs in either orientation. For example, the CD2×CD3ε iNKT-tCAR is encompasses a tCAR with the orientation CD2-CD3ε or one with the orientation CD3ε-CD2.

TABLE 4

| | Antigen Target | Antigen | |
|---|---|---|---|
| | of CAR-T or | Deletion/ | Tandem |
| Example | iNKT-CAR | Suppression | or Dual |
| 15 | CD2 × CD3ε | — | Tandem |
| 16 | CD2 × CD3ε | CD2 | Tandem |
| 17 | CD2 × CD3ε | CD3ε | Tandem |
| 18 | CD2 × CD3ε | CD2 and CD3ε | Tandem |
| 19 | CD2 × CD4 | — | Tandem |
| 20 | CD2 × CD4 | CD2 | Tandem |
| 21 | CD2 × CD4 | CD4 | Tandem |
| 22 | CD2 × CD4 | CD2 and CD4 | Tandem |
| 23 | CD2 × CD5 | — | Tandem |
| 24 | CD2 × CD5 | CD2 | Tandem |
| 25 | CD2 × CD5 | CD5 | Tandem |
| 26 | CD2 × CD5 | CD2 and CD5 | Tandem |
| 27 | CD2 × CD7 | — | Tandem |
| 28 | CD2 × CD7 | CD2 | Tandem |
| 29 | CD2 × CD7 | CD7 | Tandem |
| 30 | CD2 × CD7 | CD2 and CD7 | Tandem |
| 31 | CD3ε × CD4 | — | Tandem |
| 32 | CD3ε × CD4 | CD3ε | Tandem |
| 33 | CD3ε × CD4 | CD4 | Tandem |
| 34 | CD3ε × CD4 | CD3ε and CD4 | Tandem |
| 35 | CD3ε × CD5 | — | Tandem |
| 36 | CD3ε × CD5 | CD3ε | Tandem |
| 37 | CD3ε × CD5 | CD5 | Tandem |
| 38 | CD3ε × CD5 | CD3ε and CD5 | Tandem |
| 39 | CD3ε × CD7 | — | Tandem |
| 40 | CD3ε × CD7 | CD3ε | Tandem |
| 41 | CD3ε × CD7 | CD7 | Tandem |
| 42 | CD3ε × CD7 | CD3ε and CD7 | Tandem |
| 43 | CD4 × CD5 | — | Tandem |
| 44 | CD4 × CD5 | CD4 | Tandem |
| 45 | CD4 × CD5 | CD5 | Tandem |
| 46 | CD4 × CD5 | CD4 and CD5 | Tandem |
| 47 | CD4 × CD7 | — | Tandem |
| 48 | CD4 × CD7 | CD4 | Tandem |
| 49 | CD4 × CD7 | CD7 | Tandem |
| 50 | CD4 × CD7 | CD5 and CD7 | Tandem |
| 51 | CD5 × CD7 | — | Tandem |
| 52 | CD5 × CD7 | CD5 | Tandem |
| 53 | CD5 × CD7 | CD7 | Tandem |
| 54 | CD5 × CD7 | CD5 and CD7 | Tandem |
| 55 | TRAC × CD2 | — | Tandem |
| 56 | TRAC × CD2 | TRAC | Tandem |
| 57 | TRAC × CD2 | CD2 | Tandem |
| 58 | TRAC × CD2 | TRAC and CD2 | Tandem |
| 59 | TRAC × CD3ε | — | Tandem |
| 60 | TRAC × CD3ε | TRAC | Tandem |
| 61 | TRAC × CD3ε | CD3ε | Tandem |
| 62 | TRAC × CD3ε | TRAC and CD3ε | Tandem |
| 63 | TRAC × CD4 | — | Tandem |
| 64 | TRAC × CD4 | TRAC | Tandem |
| 65 | TRAC × CD4 | CD4 | Tandem |
| 66 | TRAC × CD4 | TRAC and CD4 | Tandem |
| 67 | TRAC × CD5 | — | Tandem |
| 68 | TRAC × CD5 | TRAC | Tandem |
| 69 | TRAC × CD5 | CD5 | Tandem |
| 70 | TRAC × CD5 | TRAC and CD5 | Tandem |
| 71 | TRAC × CD7 | — | Tandem |
| 72 | TRAC × CD7 | TRAC | Tandem |
| 73 | TRAC × CD7 | CD7 | Tandem |
| 74 | TRAC × CD7 | TRAC and CD7 | Tandem |
| 75 | TCRβ × CD2 | — | Tandem |
| 76 | TCRβ × CD2 | TCRβ | Tandem |
| 77 | TCRβ × CD2 | CD2 | Tandem |
| 78 | TCRβ × CD2 | TCRβ and CD2 | Tandem |
| 79 | TCRβ × CD3ε | — | Tandem |
| 80 | TCRβ × CD3ε | TCRβ | Tandem |
| 81 | TCRβ × CD3ε | CD3ε | Tandem |
| 82 | TCRβ × CD3ε | TCRβ and CD3ε | Tandem |
| 83 | TCRβ × CD4 | — | Tandem |
| 84 | TCRβ × CD4 | TCRβ | Tandem |
| 85 | TCRβ × CD4 | CD4 | Tandem |
| 86 | TCRβ × CD4 | TCRβ and CD4 | Tandem |
| 87 | TCRβ × CD5 | — | Tandem |
| 88 | TCRβ × CD5 | TCRβ | Tandem |
| 89 | TCRβ × CD5 | CD5 | Tandem |
| 90 | TCRβ × CD5 | TCRβ and CD5 | Tandem |
| 91 | TCRβ × CD7 | — | Tandem |
| 92 | TCRβ × CD7 | TCRβ | Tandem |
| 93 | TCRβ × CD7 | CD7 | Tandem |
| 94 | TCRβ × CD7 | TCRβ and CD7 | Tandem |
| 95 | CD2 × CD3ε | — | Dual |
| 96 | CD2 × CD3ε | CD2 | Dual |
| 97 | CD2 × CD3ε | CD3ε | Dual |
| 98 | CD2 × CD3ε | CD2 and CD3ε | Dual |
| 99 | CD2 × CD4 | — | Dual |
| 100 | CD2 × CD4 | CD2 | Dual |
| 101 | CD2 × CD4 | CD4 | Dual |
| 102 | CD2 × CD4 | CD2 and CD4 | Dual |
| 103 | CD2 × CD5 | — | Dual |
| 104 | CD2 × CD5 | CD2 | Dual |
| 105 | CD2 × CD5 | CD5 | Dual |
| 106 | CD2 × CD5 | CD2 and CD5 | Dual |
| 107 | CD2 × CD7 | — | Dual |
| 108 | CD2 × CD7 | CD2 | Dual |
| 109 | CD2 × CD7 | CD7 | Dual |
| 110 | CD2 × CD7 | CD2 and CD7 | Dual |
| 111 | CD3ε × CD4 | — | Dual |
| 112 | CD3ε × CD4 | CD3ε | Dual |
| 113 | CD3ε × CD4 | CD4 | Dual |
| 114 | CD3ε × CD4 | CD3ε and CD4 | Dual |
| 115 | CD3ε × CD5 | — | Dual |
| 116 | CD3ε × CD5 | CD3ε | Dual |
| 117 | CD3ε × CD5 | CD5 | Dual |
| 118 | CD3ε × CD5 | CD3ε and CD5 | Dual |
| 119 | CD3ε × CD7 | — | Dual |
| 120 | CD3ε × CD7 | CD3ε | Dual |
| 121 | CD3ε × CD7 | CD7 | Dual |
| 122 | CD3ε × CD7 | CD3ε and CD7 | Dual |
| 123 | CD4 × CD5 | — | Dual |
| 124 | CD4 × CD5 | CD4 | Dual |
| 125 | CD4 × CD5 | CD5 | Dual |
| 126 | CD4 × CD5 | CD4 and CD5 | Dual |
| 127 | CD4 × CD7 | — | Dual |
| 128 | CD4 × CD7 | CD4 | Dual |
| 129 | CD4 × CD7 | CD7 | Dual |
| 130 | CD4 × CD7 | CD5 and CD7 | Dual |
| 131 | CD5 × CD7 | — | Dual |
| 132 | CD5 × CD7 | CD5 | Dual |
| 133 | CD5 × CD7 | CD7 | Dual |
| 134 | CD5 × CD7 | CD5 and CD7 | Dual |
| 135 | TRAC × CD2 | — | Dual |
| 136 | TRAC × CD2 | TRAC | Dual |
| 137 | TRAC × CD2 | CD2 | Dual |
| 138 | TRAC × CD2 | TRAC and CD2 | Dual |
| 139 | TRAC × CD3ε | — | Dual |
| 140 | TRAC × CD3ε | TRAC | Dual |
| 141 | TRAC × CD3ε | CD3ε | Dual |
| 142 | TRAC × CD3ε | TRAC and CD3ε | Dual |
| 143 | TRAC × CD4 | — | Dual |
| 144 | TRAC × CD4 | TRAC | Dual |
| 145 | TRAC × CD4 | CD4 | Dual |
| 146 | TRAC × CD4 | TRAC and CD4 | Dual |
| 147 | TRAC × CD5 | — | Dual |
| 148 | TRAC × CD5 | TRAC | Dual |
| 149 | TRAC × CD5 | CD5 | Dual |
| 150 | TRAC × CD5 | TRAC and CD5 | Dual |
| 151 | TRAC × CD7 | — | Dual |
| 152 | TRAC × CD7 | TRAC | Dual |
| 153 | TRAC × CD7 | CD7 | Dual |
| 154 | TRAC × CD7 | TRAC and CD7 | Dual |
| 155 | TCRβ × CD2 | — | Dual |
| 156 | TCRβ × CD2 | TCRβ | Dual |
| 157 | TCRβ × CD2 | CD2 | Dual |
| 158 | TCRβ × CD2 | TCRβ and CD2 | Dual |
| 159 | TCRβ × CD3ε | — | Dual |
| 160 | TCRβ × CD3ε | TCRβ | Dual |
| 161 | TCRβ × CD3ε | CD3ε | Dual |
| 162 | TCRβ × CD3ε | TCRβ and CD3ε | Dual |
| 163 | TCRβ × CD4 | — | Dual |
| 164 | TCRβ × CD4 | TCRβ | Dual |
| 165 | TCRβ × CD4 | CD4 | Dual |
| 166 | TCRβ × CD4 | TCRβ and CD4 | Dual |
| 167 | TCRβ × CD5 | — | Dual |
| 168 | TCRβ × CD5 | TCRβ | Dual |
| 169 | TCRβ × CD5 | CD5 | Dual |
| 170 | TCRβ × CD5 | TCRβ and CD5 | Dual |
| 171 | TCRβ × CD7 | — | Dual |
| 172 | TCRβ × CD7 | TCRβ | Dual |
| 173 | TCRβ × CD7 | CD7 | Dual |
| 174 | TCRβ × CD7 | TCRβ and CD7 | Dual |

### Example 12—Additional Examples for Mono, Dual, and Tandem CAR-T and iNKT-CARs

Additional examples for mono, dual, and tandem CAR-T and iNKT-CARs targeting antigens expresses on multiple myeloma cells are provided below, with and without deletion or suppression of one or more surface proteins that is/are the antigen targets of the CARs and is expressed on T cells and iNKT cells. In general, examples with deletion or suppression of more antigens will be more likely to have the benefit of greater fratricide-resistance for these cells.

TABLE 5

| | Antigen Target | Antigen | Mono, |
|---|---|---|---|
| | of CAR-T and | Deletion/ | Tandem |
| Example | iNKT-CAR | Suppression | or Dual |
| 175 | BCMA | — | Mono |
| 176 | CS1 | — | Mono |
| 177 | CD19 | — | Mono |
| 178 | CD38 | — | Mono |
| 179 | CS1 | CS1 | Mono |
| 180 | CD38 | CD38 | Mono |
| 181 | APRIL | — | Mono |
| 182 | BCMA × CS1 | — | Tandem |
| 183 | BCMA × CS1 | CS1 | Tandem |
| 184 | BCMA × CD19 | — | Tandem |
| 185 | BCMA × CD38 | — | Tandem |
| 186 | BCMA × CD38 | CD38 | Tandem |
| 187 | CS1 × CD19 | — | Tandem |
| 188 | CS1 × CD19 | CS1 | Tandem |
| 189 | CS1 × CD38 | — | Tandem |
| 190 | CS1 × CD38 | CS1 | Tandem |
| 191 | CS1 × CD38 | CD38 | Tandem |
| 192 | CS1 × CD38 | CS1 and | Tandem |
| | | CD38 | |
| 193 | CD19 × CD38 | — | Tandem |
| 194 | CD19 × CD38 | CD38 | Tandem |
| 195 | APRIL × CS1 | — | Tandem |
| 196 | APRIL × CS1 | CS1 | Tandem |
| 197 | APRIL × BCMA | — | Tandem |
| 198 | APRIL × CD19 | — | Tandem |
| 199 | APRIL × CD38 | — | Tandem |
| 200 | APRIL × CD38 | CD38 | Tandem |
| 201 | BCMA | — | Dual |
| 202 | CS1 | — | Dual |
| 203 | CD19 | — | Dual |
| 204 | CD38 | — | Dual |
| 205 | BCMA | — | Dual |
| 206 | CS1 | CS1 | Dual |
| 207 | CD38 | CD38 | Dual |
| 208 | BCMA × CS1 | — | Dual |
| 209 | BCMA × CS1 | CS1 | Dual |
| 210 | BCMA × CD19 | — | Dual |
| 211 | BCMA × CD38 | — | Dual |
| 212 | BCMA × CD38 | CD38 | Dual |
| 213 | CS1 × CD19 | — | Dual |
| 214 | CS1 × CD19 | CS1 | Dual |
| 215 | CS1 × CD38 | — | Dual |
| 216 | CS1 × CD38 | CS1 | Dual |
| 217 | CS1 × CD38 | CD38 | Dual |
| 218 | CS1 × CD38 | CS1 and | Dual |
| | | CD38 | |
| 219 | CD19 × CD38 | — | Dual |
| 220 | CD19 × CD38 | CD38 | Dual |
| 221 | APRIL × CS1 | — | Dual |
| 222 | APRIL × CS1 | CS1 | Dual |
| 223 | APRIL × BCMA | — | Dual |
| 224 | APRIL × CD19 | — | Dual |
| 225 | APRIL × CD38 | — | Dual |
| 226 | APRIL × CD38 | CD38 | Dual |

### Example 13—Gene-Edited T Cells and iNKT Cells without CARs

TABLE 6

| | Surface Protein |
|---|---|
| | (Antigen) |
| Example | Deletions |
| 231 | CD2 |
| 232 | CD3ε |
| 233 | CD4 |
| 234 | CD5 |
| 235 | CD7 |
| 236 | TRAC |
| 237 | TCRβ |
| 238 | CD2 and CD3ε |
| 239 | CD2 and CD4 |
| 240 | CD2 and CD5 |
| 241 | CD2 and CD7 |
| 242 | CD3ε and CD4 |
| 243 | CD3ε and CD5 |
| 244 | CD3ε and CD7 |
| 245 | CD4 and CD5 |
| 246 | CD4 and CD7 |
| 247 | CD5 and CD7 |
| 248 | TRAC and CD2 |
| 249 | TRAC and CD3 |
| 250 | TRAC and CD4 |
| 251 | TRAC and CD5 |
| 252 | TRAC and CD7 |
| 253 | TCRβ and CD2 |
| 254 | TCRβ and CD3 |
| 255 | TCRβ and CD4 |
| 256 | TCRβ and CD5 |
| 257 | TCRβ and CD7 |
| 258 | BCMA |
| 259 | CS1 |
| 260 | CD19 |
| 261 | CD3ε |
| 262 | BCMA × CS1 |
| 263 | BCMA × CD19 |
| 264 | BCMA × CD38 |
| 265 | CS1 × CD19 |
| 266 | CS1 × CD38 |
| 267 | CD19 × CD38 |

### Example 14—Efficacy of BCMA-CAR-iNKT Cells and NT-I7 Combination in the Treatment of Multiple Myeloma

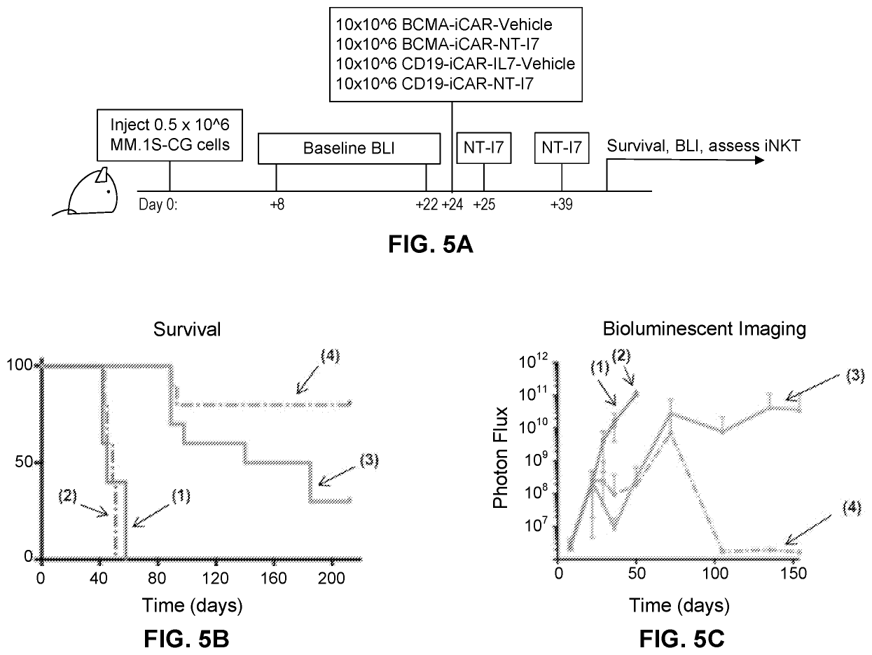

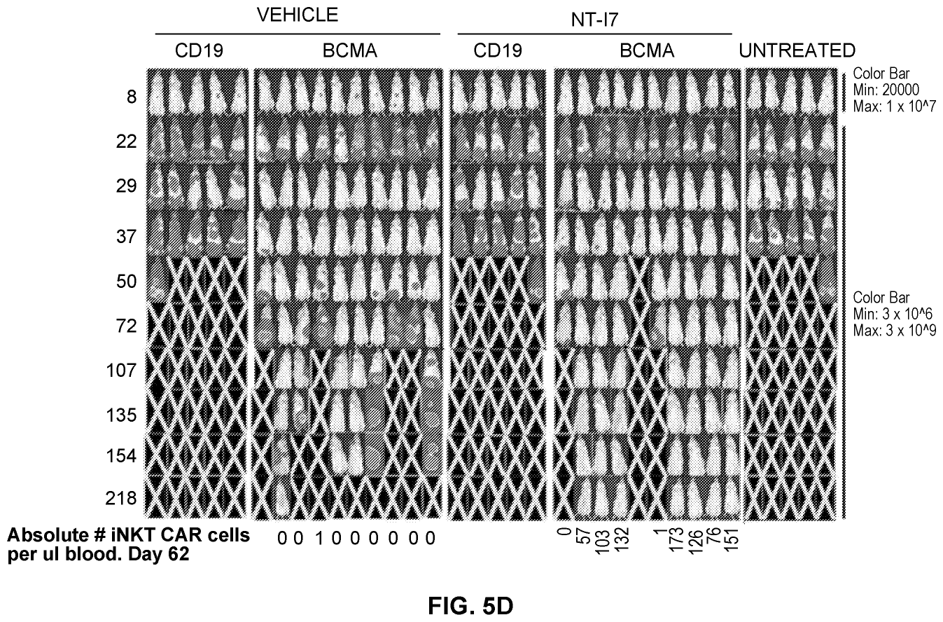

The effect of BCMA-CAR-iNKT cells (both with vehicle or in combination with NT-I7) on multiple myeloma was tested in a mouse model. Briefly, NSG mice were injected with MM.1S-C/G-Luciferase tumor cells (5×10⁵ cells/mouse) and 24 days later were treated with nothing, 10×10≢CD19-CAR-iNKT, or 10×10≢BCMA-CAR-iNKTs). Mice that were treated with CAR-iNKTs were subsequently treated with vehicle or NT-I7 (day 25, and day 39). For comparison purposes, some of the animals were treated with CD19-CAR-iNKT cells with vehicle or in combination with NT-I7. One set of mice was treated with nothing. As shown in FIG. 5A, compared to those animals treated with the other treatment regimens, greater number of the tumor mice treated with the BCMA-iNKT-CAR cells in combination with NT-I7 survived the entire duration of the experiment (i.e., 218 days post tumor inoculation).

In agreement with the survival data, animals treated with BCMA-CAR-INKT cells in combination with NT-I7 had significantly reduced tumor burden by the end of the experiment (i.e., 135 days post tumor inoculation). See FIG. 5B, “(4).” Interestingly, among animals treated with BCMA-CAR-iNKT cells alone, tumor burden remained high in some of the mice at the end of the experiment while all surviving BCMA-CAR-iNKT mice had background levels of tumor burden. See FIG. 5C, “(3).” Bioluminescent imaging (FIG. 5D) suggests that by about day 29 post tumor inoculation, majority of animals treated with BCMA-CAR-iNKT cells (alone or in combination with NT-I7) were nearly tumor-free. However, some of the animals treated with BCMA-CAR-iNKT cells plus vehicle began to exhibit higher tumor burden at later time points.

### Example 15—Efficacy of NT-I7 on Enhancement of Anti-Tumor Activity of CAR T Cells in Relapsed Animals

As described in Example 14, some of the animals treated with BCMA iNKT CAR T cells alone appeared to initially control tumor growth, but then subsequently, showed disease progression (as evidenced by tumor burden using bioluminescent imaging). Therefore, whether subsequent administration of NT-I7 into these animals can enhance the anti-tumor activity of the BCMA iNKT CAR T cells will be assessed. Briefly, NSG mice will be injected with MM.1S-C/G-Luciferase tumor cells (5×10⁵ cells/mouse). Then, the animals will be treated with iNKT-BCMA CAR T cells, alone or in combination with NT-I7. After tumor relapse, the animals will receive additional doses of NT-I7. Both survival and tumor burden will be monitored.

### Example 16—Efficacy of NT-I7 on Enhancement of Anti-Tumor Activity of CAR T Cells Late After Tumor Establishment

In approximately 50% of myeloma tumor bearing mice treated with CS1 CAR T cells alone develop extramedullary tumors around day 100 post tumor inoculation. Whether administration of NT-I7 at this stage of disease progression can enhance anti-tumor activity of the CS1 CAR T cells will be assessed. Briefly, NSG mice will be injected with MM.1S-C/G-Luciferase tumor cells (5×10⁵ cells/mouse). Then, the animals will be treated with CS1 CAR T cells as described in Example 16. In animals that develop extramedullary tumors at around day 100, NT-I7 will be administered. Animal survival and tumor burden will be periodically monitored.

### Example 17—Efficacy of NT-I7 on Enhancing Anti-Tumor Activity of Anti-CD33 CAR T Cells and Anti-CLL1 CAR T Cells Against Acute Myeloid Leukemia (AML)

The effect of NT-I7 on the anti-tumor activity of anti-CD33 CAR T cells (CART33) and anti-CLL1 CAR T cells (CART371) will be assessed in a mouse model of acute myeloid leukemia. Briefly, NSG mice will receive U937CBR-GFP tumor cells that express both CD33 and CLL-1 on their surface (5×10⁴ cells/mouse). At around days 5-7 post tumor inoculation, bioluminescence imaging (BLI) will be used to confirm tumor engraftment. Then, the animals with established tumors will receive one of the following groups of T cells: (i) untransduced; (ii) CART33; and (iii) CART371. Appropriate groups of animals will then receive subcutaneous administration of NT-I7 (10 mg/kg) on days 8, 22, and 36 post tumor inoculation. The animals will be followed weekly via BLI to monitor tumor burden, and peripheral blood flow cytometry will be performed to monitor T cell expansion. Table 7 (below) provides the different treatment groups.

TABLE 7
Treatment Groups

| Group | Treatment Regimen |
|---|---|
| G1 | No treatment (control) |
| G2 | U937^{CBR-GFP} tumor cells + untransduced T cells |
| G3 | U937^{CBR-GFP} tumor cells + untransduced T cells + NT-17 |
| G4 | U937^{CBR-GFP} tumor cells + CART33 |
| G5 | U937^{CBR-GFP} tumor cells + CART33 + NT-17 |
| G6 | U937^{CBR-GFP} tumor cells + CART371 |
| G7 | U937^{CBR-GFP} tumor cells + CART371 + NT-17 |

### Example 18—Efficacy of CLL-1 CAR T Cells and NT-I7 Combination in the Treatment of Acute Myeloid Leukemia

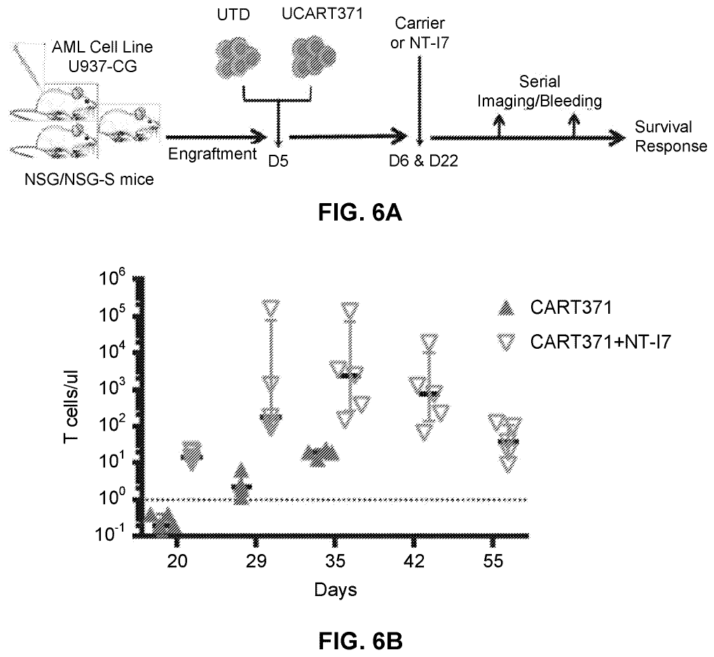

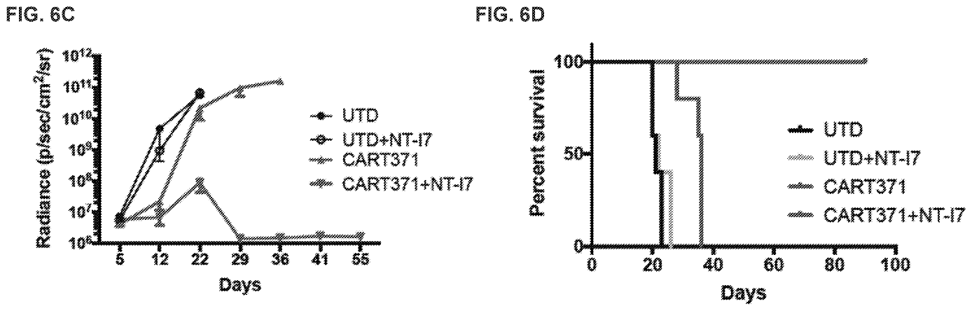

To assess the effect of CLL-1 CAR T cells, alone and in combination with NT-I7, on acute myeloid leukemia (AML), NSG/NSG-S mice were inoculated with the AML cell line U937-CG. FIG. 6A. At five days post-tumor inoculation, animals were treated with untransduced (UTD) CD3 knock-out T cells or received an administration of universal CLL-1 CAR T cells (UCART371). Then, at days 6 and 22 post-tumor inoculation, the animals received either a carrier control or NT-I7. The treatment groups were the following: (i) untransduced (UTD) CD3 knock-out T cells, (ii) untransduced (UTD) CD3 knock-out T cells with NT-I7, (iii) UCART371 alone, and (iv) both UCART371 and NT-I7. Animals from each of the groups were monitored at various time points for human T cell expansion (in blood), tumor burden, and survival.

As shown in FIG. 6B, animals that received both UCART371 and NT-I7 had significantly higher number of T cells in the peripheral blood compared to animals that received only UCART371. Similarly, animals from the combination group had significantly reduced tumor burden (compared to the other treatment groups) and survived the entire duration of the experiment. FIGS. 6C and 6D, respectively. In contrast, animals treated with UCART371 alone had improved tumor immune response compared to both the T cell only and the T cells with NT-I7 treated groups. However, all the animals from the UCART371 alone group succumbed to the tumor by about day 36 post-tumor inoculation.

### Example 19—Analysis of the Effect of NT-I7 on CAR T Cells after Tumor Re-Challenge

To assess whether the administration of NT-I7 can also improve the ability of the CAR T cells to respond to secondary tumor challenge, the seven mice initially treated with BCMA-CAR-iNKT cells in combination with NT-17 (see Example 14) and had no tumor burden by day 218 (see FIG. 5D) were re-challenged with 5×10⁵ MM.1S-CG cells. Then, three of mice the mice received vehicle control, and four of the mice were treated with a second course of NT-I7 administration. As a positive control, five NSG mice (never received prior treatment) were treated with tumor and vehicle control at the same time. Animals were monitored over a ten-week period for tumor burden (via bioluminescent imaging) and the number of CAR-iNKT cells in the blood.

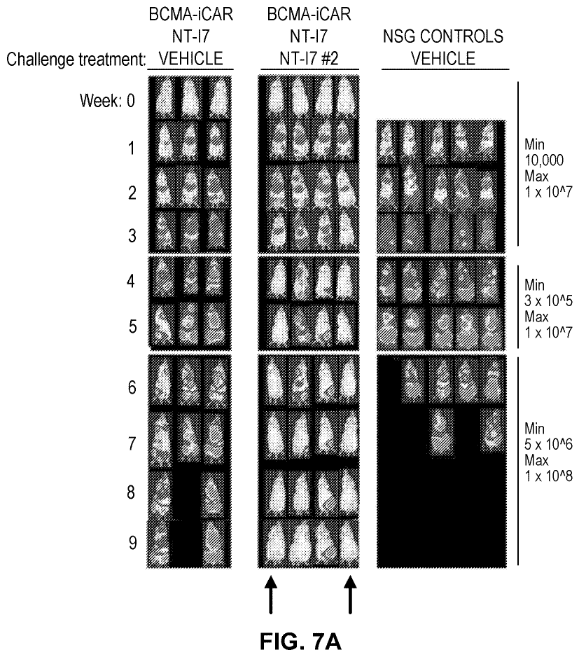

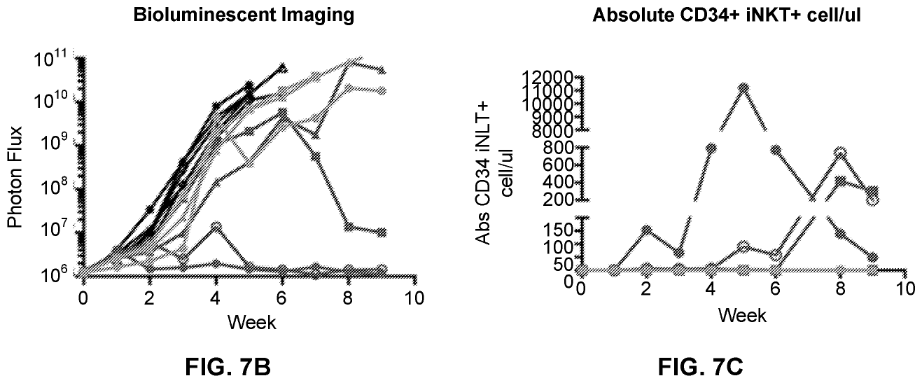

As seen in FIGS. 7A and 7B, all of the mice that received both BCMA-CAR-iNKT cells and NT-I7 survived to the end of the experiment, with three of the four mice being tumor free. In contrast, among animals that only received the CAR T cells, two out of three survived, and they all had extensive tumor burden. Moreover, only those animals that also received the NT-I7 administration had detectable levels of BCMA-CAR-iNKT cells in the blood. (FIG. 7C)

Collectively, the above results demonstrate that NT-I7 can improve the anti-tumor effects of CAR T cells (both to primary and secondary challenges) to various tumors.

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

1. A method for treating a cancer in a subject in need thereof, comprising administering to the subject concurrently or sequentially,
a. a population of chimeric antigen receptor (CAR)-bearing immune effector cells, and
b. an interleukin-7 (IL-7) fusion protein,
wherein the IL-7 fusion protein comprises an IL-7 protein and a half-life extending moiety.

2. The method of claim 1, wherein the IL-7 protein comprises an amino acid sequence that has at least about 70% identical sequence identity to the amino acid sequence set forth in SEQ ID NO. 1.

3-6. (canceled)

7. The method of claim 1, wherein the half-life extending moiety is selected from the group consisting of an Fc region of immunoglobulin or a part thereof (Fc domain), albumin, an albumin binding polypeptide, Pro/Ala/Ser (PAS), C-terminal peptide(CTP) of β subunit of human chorionic gonadotropin, polyethylene glycol (PEG), long unstructured hydrophilic sequences of amino acids (XTEN), hydroxyethyl starch (HES), an albumin-binding small molecule, and a combination thereof.

8-9. (canceled)

10. The method of claim 1, wherein the IL-7 fusion protein comprises an amino acid sequence that has at least about 70% sequence identity to the amino acid sequence set forth in any one of SEQ ID NOs: 21-26.

11. The method of claim 1, wherein the IL-7 fusion protein is administered to the subject (i) at a weight-based dose of between about 20 μg/kg and about 2,000 μg/kg or at a flat dose of about 0.25 mg to about 9 mg; (ii) at a dosing interval of at least about one week: or (iii) both (i) and (ii)

12-24. (canceled)

25. The method of claim 1, wherein the IL-7 fusion protein is capable of achieving one or more of:
(i) increased expansion of,
(ii) increased persistence of, and/or
(iii) increased anti-tumor activity of
the population of CAR-bearing immune effector cells.

26-33. (canceled)

34. The method of claim 1, wherein the CAR-bearing immune effector cells comprise CAR-T cells, CAR-bearing iNKT cells (iNKT-CAR), or both.

35. (canceled)

36. The method of claim 1, wherein the CAR targets one or more antigens selected from CD2, CD3c, CD4, CD5, CD7, CD19, TRAC, BCMA, TCRβ, or combinations thereof.

37. The method of claim 36, wherein the CAR-bearing immune effector cells are genome-edited.

38. The method of claim 37, wherein the genome-edited CAR-bearing immune effector cells comprise a deletion or modification in one or more antigens selected from CD2, CD3c, CD4, CD5, CD7, TRAC, BCMA, TCRβ, or combinations thereof.

39-48. (canceled)

49. The method of claim 1, wherein the cancer comprises a solid tumor.

50. (canceled)

51. The method of claim 1, wherein the cancer is hematologic malignancy.

52-65. (canceled)

66. The method of claim 1, wherein the subject is further administered an anti-cancer agent, a lymphocyte depleting agent, or both.

67-85. (canceled)

86. A method of increasing a survival and/or an expansion of a population of chimeric antigen receptor (CAR)-bearing immune effector cells in a subject in need thereof, comprising administering to the subject an interleukin-7 (IL-7) fusion protein and a population of CAR-bearing immune effector cells, wherein the IL-7 fusion protein comprises an IL-7 protein and a half-life extending moiety, and wherein, after the administering, the survival and/or the expansion of the population of CAR-bearing immune effector cells is increased as compared to a reference method comprising administering the population of CAR-bearing immune effector cells without the IL-7 fusion protein.

87. A method of improving an anti-tumor activity of a population of chimeric antigen receptor (CAR)-bearing immune effector cells in a subject in need thereof, comprising administering to the subject an interleukin-7 (IL-7) fusion protein and a population of chimeric antigen receptor (CAR)-bearing immune effector cells, and wherein the IL-7 fusion protein comprises an IL-7 protein and a half-life extending moiety.

88. The method of claim 1, wherein the IL-7 fusion protein further comprises an oligopeptide consisting of 1 to 10 amino acid residues.

89. The method of claim 88, wherein the oligopeptide is selected from the group consisting of methionine, glycine, methionine-methionine, glycine-glycine, methionine-glycine, glycine-methionine, methionine-methionine-methionine, methionine-methionine-glycine, methionine-glycine-methionine, glycine-methionine-methionine, methionine-glycine-glycine, glycine-methionine-glycine, glycine-glycine-methionine, and glycine-glycine-glycine.

90. (canceled)

91. The method of claim 1, wherein the IL-7 fusion protein is administered to the subject: (i) less than about six months after administering the population of CAR-bearing immune effector cells, or (ii) at least about one day before administering the population of CAR-bearing immune effector cells.

92-96. (canceled)

97. The method of claim 86, wherein the half-life extending moiety is selected from the group consisting of an Fc region of immunoglobulin or a part thereof (Fc domain), albumin, an albumin binding polypeptide, Pro/Ala/Ser (PAS), C-terminal peptide(CTP) of β subunit of human chorionic gonadotropin, polyethylene glycol (PEG), long unstructured hydrophilic sequences of amino acids (XTEN), hydroxyethyl starch (HES), an albumin-binding small molecule, and a combination thereof

98. The method of claim 97, wherein the half-life extending moiety is an Fc domain.

99-103. (canceled)
